(19) United States
(12) Patent Application Publication
(10) Pub. No.: US 2025/0249001 A1
(43) Pub. Date: Aug. 7, 2025

(54) METHODS OF TREATING SUBSTANCE USE DISORDER WITH 4-(3-CYANOPHENYL)-6-PYRIDINYLPYRIMIDINE MGLU5 NEGATIVE ALLOSTERIC MODULATORS

(71) Applicant: Tempero Bio, Inc., Oakland, CA (US)

(72) Inventors: Bruce IMBERT, Novato, CA (US); Victoria SUNG, San Francisco, CA (US); Ricardo E. DOLMETSCH, Concord, MA (US); Clyde W. HODGE, Chapel Hill, NC (US)

(21) Appl. No.: 18/999,857

(22) Filed: Dec. 23, 2024

Related U.S. Application Data
(63) Continuation of application No. PCT/US2023/023586, filed on May 25, 2023.
(60) Provisional application No. 63/355,451, filed on Jun. 24, 2022.

(51) Int. Cl. A61K 31/506; A61P 25/32

(52) U.S. Cl. CPC A61K 31/506; A61P 25/32

(57) ABSTRACT

The present disclosure relates to methods for treating subjects having a substance use disorder (SUD) with 4-(3-cyanophenyl)-6-pyridinylpyrimidine mGlu5 negative allosteric modulators. Specifically, in some embodiments, this disclosure relates to methods of treating a subject having SUD who is at risk of relapse to alcohol use. In some embodiments, this disclosure relates to methods of promoting remission in a subject having SUD. In some embodiments, the SUD is alcohol use disorder (AUD), opioid use disorder (OUD), or a stimulant use disorder such as cocaine use disorder (CUD).

### CROSS-REFERENCE TO RELATED APPLICATIONS

This application is a continuation of International Patent Application No. PCT/US2023/023586, filed May 25, 2023, which claims priority to U.S. Provisional Application No. 63/355,451, filed Jun. 24, 2022, each of which is herein incorporated by reference in its entirety for all purposes.

### STATEMENT OF GOVERNMENT INTEREST

This invention was made with government support under Contract Number DA057118 awarded by the National Institutes of Health. The government has certain rights in the invention.

### FIELD OF DISCLOSURE

The present disclosure relates to methods for treating subjects having a substance use disorder with 4-(3-cyanophenyl)-6-pyridinylpyrimidine mGlu5 negative allosteric modulators. Specifically, in some embodiments, this disclosure relates to methods of treating a subject having SUD who is at risk of relapse to alcohol use. In some embodiments, this disclosure relates to methods of promoting remission in a subject having SUD. In some embodiments, the SUD is alcohol use disorder (AUD), opioid use disorder (OUD), or a stimulant use disorder such as cocaine use disorder (CUD).

### BACKGROUND

Current evidence shows that medications are underused in the treatment of substance use disorders such as alcohol use disorder (AUD), opioid use disorder (OUD), and stimulant use disorders such as cocaine use disorder (CUD). This is of concern because of the high prevalence of alcohol problems in the general population. For example, in the United States, the 12-month prevalence of AUD is estimated to be 4.6% among 12- to 17-year-olds and 8.5% among adults aged 18 years and older in the United States. Rates of the disorder are greater among adult men (12.4%) than among adult women (4.9%). Problems with alcohol influence the incidence, course, and treatment of many other medical and psychiatric conditions. Many experts in addiction believe that patients with moderate or severe substance related problems, such as alcohol-related problems, should be offered medication-assisted treatment on a routine basis.

The development of an SUD (e.g., AUD, OUD, CUD) is a multiphasic process characterized initially by repeated usage episodes that give way to end stages of addiction that include dependence/withdrawal syndromes. Relapse after abstinence is a complex phenomenon that involves more than physical compulsion to use a substance. It is often associated with anxiety and dysphoria secondary to downregulation of dopamine signaling and can be triggered by environmental cues like stress and negative emotions. Medications used to treat substance use disorder may not be efficacious for preventing relapse. For example, Methadone and Buprenorphine which act as agonists at the opiate receptor can be used to substitute for the drug abuse and can help to reduce substance use in individuals with SUD. Similarly, Acamprosate, a medication used to treat alcohol use disorder, reduces overall alcohol use. However, Methadone, Buprenorphine and Acaprosate are not effective in preventing relapse following detoxification. Similarly, medications used to prevent or treat relapse to a substance after abstinence are not necessarily effective for reducing consumption of the substance. For example, naltrexone, an opiate antagonist, is somewhat effective at reducing relapse in people with alcohol use disorder but is much less effective in people currently using alcohol.

The reinforcing, or rewarding, effects of the substance are a factor of abuse liability and may drive the development and maintenance of SUD. Thus, methods that reduce the reinforcing properties of substances (e.g., alcohol, opioids, cocaine) could be useful to the development of pharmacotherapies to treat SUDs in the clinic. Similarly, relapse to substance-seeking after abstinence is a factor in the behavioral pathology of SUD that may be triggered by conditioned reinforcement (cue-induced) processes. Accordingly, new methods are needed for reducing substance use relapse after abstinence, and methods are also needed for mitigating substance use triggers in SUD patients.

### SUMMARY

The present disclosure provides a method of treating a subject having a substance use disorder (SUD) who is at risk of relapse to substance use, the method comprising administering to the subject a therapeutically effective amount of a compound of the formula

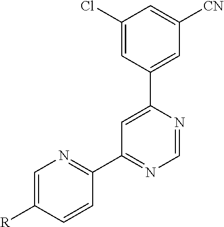

or a pharmaceutically acceptable salt thereof, wherein R is H, Me, F, Cl or cyano;

wherein the administration reduces susceptibility to one or more relapse triggers. In some embodiments, the SUD is alcohol use disorder (AUD), opioid use disorder (OUD), or a stimulant use disorder such as cocaine use disorder (CUD). In some embodiments, the SUD is AUD.

The present disclosure also provides a method of promoting remission in a subject having an SUD, the method comprising administering to the subject a therapeutically effective amount of a compound of the formula

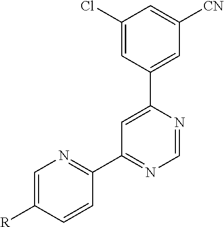

or a pharmaceutically acceptable salt thereof, wherein R is H, Me, F, Cl or cyano;

wherein the administration results in remission for at least 3 months. In some embodiments, the SUD is AUD, OUD, and/or a stimulant use disorder (e.g., CUD). In some embodiments, the SUD is AUD.

The present disclosure also provides a use of a compound in a treatment for reducing susceptibility to one or more relapse triggers, wherein the treatment comprises administering a therapeutically effective amount of the compound to a subject having an SUD who is at risk of relapse to substance use;

wherein the compound is of the formula

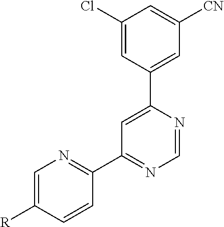

or a pharmaceutically acceptable salt thereof, wherein R is H, Me, F, Cl or cyano. In some embodiments, the SUD is AUD, OUD, and/or a stimulant use disorder (e.g., CUD). In some embodiments, the SUD is AUD.

The present disclosure also provides a use of a compound in the manufacture of a medicament for treatment for reducing susceptibility to one or more relapse triggers, wherein the treatment comprises administering the medicament to a subject having an SUD who is at risk of relapse to substance use;

wherein the compound is of the formula

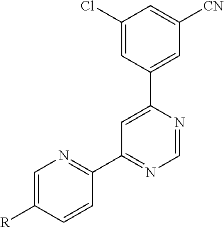

or a pharmaceutically acceptable salt thereof, wherein R is H, Me, F, Cl or cyano. In some embodiments, the SUD is AUD, OUD, and/or a stimulant use disorder (e.g., CUD). In some embodiments, the SUD is AUD.

The present disclosure also provides a compound for use in a method of reducing susceptibility to one or more relapse triggers, the method comprising administering a therapeutically effective amount of the compound to a subject having an SUD who is at risk of relapse to substance use;

wherein the compound is of the formula

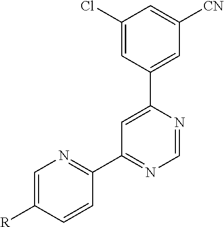

or a pharmaceutically acceptable salt thereof, wherein R is H, Me, F, Cl or cyano. In some embodiments, the SUD is AUD, OUD, and/or a stimulant use disorder (e.g., CUD). In some embodiments, the SUD is AUD.

The present disclosure also provides a medicament for treatment for reducing susceptibility to one or more relapse triggers, the medicament comprising a compound, wherein the treatment comprises administering the medicament to deliver a therapeutically effective amount of the compound to a subject having and SUD who is at risk of relapse to substance use;

wherein the compound is of the formula

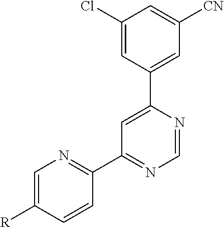

or a pharmaceutically acceptable salt thereof, wherein R is H, Me, F, Cl or cyano. In some embodiments, the SUD is AUD, OUD, and/or a stimulant use disorder (e.g., CUD). In some embodiments, the SUD is AUD.

The present disclosure also provides a use of a compound in the manufacture of a medicament for treatment for promoting remission of an SUD, wherein the treatment comprises administering the medicament to a subject having the SUD;

wherein the compound is of the formula

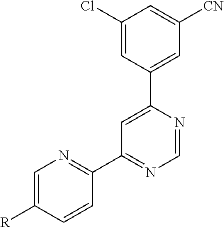

or a pharmaceutically acceptable salt thereof, wherein R is H, Me, F, Cl or cyano. In some embodiments, the SUD is AUD, OUD, and/or a stimulant use disorder (e.g., CUD). In some embodiments, the SUD is AUD.

The present disclosure also provides a use of a compound in a treatment for promoting remission of an SUD, wherein the treatment comprises administering a therapeutically effective amount of the compound to a subject having the SUD;

wherein the compound is of the formula

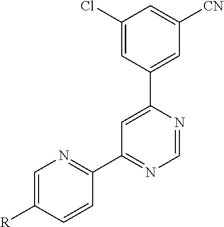

or a pharmaceutically acceptable salt thereof, wherein R is H, Me, F, Cl or cyano. In some embodiments, the SUD is AUD, OUD, and/or a stimulant use disorder (e.g., CUD). In some embodiments, the SUD is AUD.

The present disclosure also provides a compound for use in a method for promoting remission of an SUD, the method comprising administering a therapeutically effective amount of the compound to a subject having the SUD;

wherein the compound is of the formula

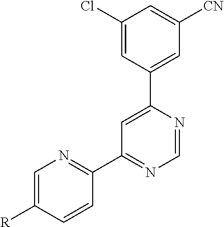

or a pharmaceutically acceptable salt thereof, wherein R is H, Me, F, Cl or cyano. In some embodiments, the SUD is AUD, OUD, and/or a stimulant use disorder (e.g., CUD). In some embodiments, the SUD is AUD.

The present disclosure also provides a medicament for treatment for promoting remission of an SUD, the medicament comprising a compound, wherein the treatment comprises administering the medicament to deliver a therapeutically effective amount of the compound to a subject having the SUD;

wherein the compound is of the formula

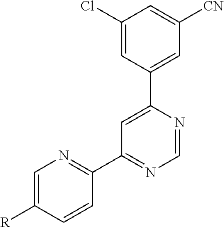

or a pharmaceutically acceptable salt thereof, wherein R is H, Me, F, Cl or cyano. In some embodiments, the SUD is AUD, OUD, and/or a stimulant use disorder (e.g., CUD). In some embodiments, the SUD is AUD.

### BRIEF DESCRIPTION OF THE DRAWINGS

FIG. 1 shows the effects of acute Compound 1 administration on voluntary home-cage alcohol drinking by Wistar rats after 1-hr or 24-hrs access. (FIG. 1A) Assessment of Compound 1 effects on alcohol intake after 1-hr of alcohol access. Graph shows mean EtOH intake (g/kg/1-h) plotted as a function of Compound 1 dosage. The three highest doses of Compound 1 (1, 3, and 10 mg/kg) each significantly decreased alcohol intake. (FIG. 1B) Positive control experiment showing mean EtOH intake (g/kg) following vehicle (0) and Compound 1 (3 mg/kg), MPEP (3 mg/kg), or Naltrexone (3 mg/kg). All 3 compounds decreased alcohol drinking at 1-hr. (FIG. 1C) Evaluation of Compound 1 effects on alcohol drinking after 24-hrs of access. Results showed that Compound 1 (1 and 3 mg/kg) each reduced alcohol intake for the full 24-hr measurement period. (FIG. 1D) Positive control experiment comparing Compound 1, MPEP and Naltrexone after 24-hrs of access. Only Compound 1 (3 mg/kg) reduced sweetened alcohol intake at the 24-hr measurement interval. All data are plotted as MEAN±SEM from N=11 rats. Asterisks indicate significantly different from vehicle control: *—P<0.05, **—P<0.01, ***—P<0.001, Šídák's multiple comparisons test.

FIG. 2 shows water intake (1-hr and 24-hr access) following treatment with Compound 1 (0-10 mg/kg) or Compound 1 (3 mg/kg) in comparison to MPEP and Naltrexone. Trends toward reduced water intake did not reach statistical significance (FIG. 2A and FIG. 2B). At the 24-hr measurement interval, RM-ANOVA identified an overall reduction in H₂O intake following pretreatment with the full dose range of Compound 1 (0-10 mg/kg) (F (5, 50)=6.542, P<0.0001). However, follow up multiple comparisons did not identify a significant change at any dose of Compound 1 as compared to vehicle control (FIG. 2C). This suggests that overall trends (both slight increases and decreases) with little variability contributed to the main effect. Finally, there was no effect of Compound 1 (3 mg/kg) or comparison compounds on H₂O intake after 24-hr access (FIG. 2D).

FIG. 3 shows Compound 1 had no effects on motor activity. (FIG. 3A) Schematic of the open-field. (FIG. 3B) Mean ambulatory distance (cm) plotted as a function of time (min) following Compound 1 (0 or 3 mg/kg) injection. Results show normal exploration and an exponentially decreasing curve as habituation occurs to the environment. Total (mean) ambulatory distance (FIG. 3C) and ambulatory time (FIG. 3D) were also unchanged by administration of Compound 1. All data are plotted as MEAN±SEM from N=11 rats.

FIG. 4 shows Compound 1 had no effects on activity in the center of an open-field. (FIG. 4A) Schematic of the open-field showing the total field and center zone. Total ambulatory distance (FIG. 4B) and ambulatory time (FIG. 4C) plotted as a function of Compound 1 (0 or 3 mg/kg) dosage were unchanged by administration of Compound 1. All data are plotted as MEAN±SEM from N=11 rats.

FIG. 5 shows an A-B-A experimental design showing effects of repeated Compound 1 (3 mg/kg) administration on voluntary home-cage alcohol drinking by Wistar rats after 1-hr or 24-hrs access. (FIG. 5A) Assessment of Compound 1 effects on alcohol intake after 1-hr of alcohol access. Graph shows mean EtOH intake (g/kg/1-h) plotted as a function of treatment day. Vehicle control was administered on days 1-4 (Veh-1), Compound 1 (3 mg/kg) on days 5-11, and vehicle control again on days 12-15 (Veh-2). Multiple comparison procedure conducted after RM-ANOVA showed that Compound 1 significantly reduced alcohol drinking only on the first day of drug administration (day 5), P<0.05. (FIG. 5B) This panel shows average alcohol drinking during each experimental phase after 1 hour of access to alcohol. Holm-Šídák's multiple comparisons test showed that average alcohol intake during the 7-day treatment phase with Compound 1 was significantly different than both vehicle conditions, P<0.05. (FIG. 5C) Evaluation of Compound 1 effects on alcohol drinking after 24-hrs of access. Holm-Šídák's multiple comparisons test showed that Compound 1 (3 mg/kg) decreased alcohol intake only on day 5 as compared to vehicle day 4, P<0.05. (FIG. 5D) No changes were observed following Compound 1 treatment when 24-hr intake data were averaged over the 3 treatment conditions. All data are plotted as MEAN±SEM from N=11 rats. Asterisks indicate significantly different from vehicle control: *—P<0.05.

FIG. 6 shows parallel control measurements (A-B-A experimental design) demonstrating that repeated treatment with Compound 1 (3 mg/kg) produced minimal effects on home-cage water (H₂O) intake. (A-B) H₂O intake plotted as a function of treatment day (FIG. 6A) or averaged across each experimental phase following 1-hr access (FIG. 6B). Panels (FIG. 6C-FIG. 6D) show H₂O intake under corresponding conditions following 24-hr access. All data are plotted as MEAN±SEM from N=11 rats. Asterisks indicate points that are significantly different from control: *—P<0.05; **—P<0.01; ***—P<0.001 via Holm-Šídák's multiple comparisons test.

FIG. 7 shows acute Compound 1 exhibited preclinical efficacy for reducing the positive reinforcing effects of EtOH as modeled by operant self-administration. (FIG. 7A) Drawing of computer-controlled operant conditioning chamber in which rats lever press on a Fixed-ratio 1 schedule of EtOH reinforcement. (FIG. 7B). Compound 1 (0-10 mg/kg, IP) administration 1-h before self-administration sessions produced a dose-dependent decrease in EtOH reinforced responses. (FIG. 7C) Compound 1 showed equivalent efficacy to reduce EtOH reinforced lever press responses when compared to the FDA approved opiate antagonist Naltrexone, with improved dose-dependent efficacy as compared to the comparable mGluR5 inhibitor MPEP. All data are plotted as MEAN±SEM from N=11 rats. Drug doses were administered in randomized order. Asterisks indicate points that are significantly different from control: *—P<0.05; **—P<0.01 via Dunnett's or Šídák's multiple comparisons test where appropriate.

FIG. 8 shows repeated daily treatment with Compound 1 (10 mg/kg) administered in a palatable oral pellet had no effect on EtOH reinforced lever press responses in an A-B-A (vehicle-Compound 1-vehicle) experiment. (FIG. 8A) Mean±SEM EtOH reinforced lever press responses plotted as a function of treatment day during vehicle administration (Day 1-5) showed no statistically significant change following repeated oral Compound 1 administration (Day 6-12). Return to vehicle treatment was also associated with no change in behavior (Day 13-17). (FIG. 8B) Summary of EtOH reinforced lever press responses calculated as an average of each rat's performance during each experimental phase. Compound 1 produced no statistically significant difference in overall mean performance when administered to rats via an oral pellet.

FIG. 9 shows that Compound 1 (10 mg/kg) significantly decreased cue-induced reinstatement of alcohol-seeking behavior. (FIG. 9A) Diagram of the self-administration chambers showing 3 behavioral phases: baseline self-administration, extinction, and reinstatement. Cues were present during self-administration and reinstatement test. EtOH was available only during the self-administration phase. (FIG. 9B). Extinction of EtOH-reinforced lever pressing. Data are plotted as mean (±SEM) responses per session on the ethanol (active) and inactive levers on the last day of baseline (B) and through the 7 days of extinction training. Data represent average performance of (N=11) rats. *—significantly different from baseline (B), P<0.0001; †—significantly different from inactive lever at specific day, P<0.01 via Šídák's multiple comparisons test. (FIG. 9C) Effect of Compound 1 (10 mg/kg) on cue-induced reinstatement of alcohol-seeking behavior. Data are shown as mean (±SEM) total responses on day 8 of the procedure (e.g., the reinstatement test day), *—significantly different from extinction (EXT), P<0.05, which indicates a robust cue-induced reinstatement of alcohol-seeking behavior following vehicle administration. Note that lever press responding after Compound 1 is not different from extinction, which indicates blockade of reinstatement. (FIG. 9D) Lever press responding shown as a relative change from extinction with each rat serving as its own control. Average individual performance on the active lever was ˜400% of extinction following vehicle injection; however, Compound 1 significantly blocked this relative increase, *—different from vehicle (VEH), P<0.05 via Šídák's multiple comparison test.

FIG. 10 shows the percent of time in the drug-paired compartment on days 1 (baseline) and 10 (bias test). One-way ANOVA with Dunnett's post hoc test, *p<0.05, ** p<0.01 vs Cocaine 10 mg/kg-Saline. Data: mean±SEM.

FIG. 11 shows the time difference on days 1 (baseline) and 10 (bias test). One-way ANOVA with Dunnett's post hoc test, *p<0.05, ** p<0.01 vs Cocaine 10 mg/kg-Saline. Data: mean±SEM.

FIG. 12 shows results of acquisition of cocaine self-administration. Rats' cocaine self-administration increased steadily in first 8 days of training, and a stable self-administration was achieved after 20 days of cocaine training. Data represent the means±SEM of number of infusions.

FIG. 13 shows the effects of Compound 1 (1 mg/kg, 3 mg/kg & 10 mg/kg), and MTEP (3 mg/kg) on cocaine self-administration in rats. Data are presented as mean±SEM. Asterisks (***: P<0.001) indicate a significant difference relative to saline treatment. N=15-16 in each treatment group.

FIG. 14 shows the effects of Compound 1 (3 mg/kg) and MTEP (3 mg/kg) on the reinstatement of a cocaine self-administration in rats (a model for relapse). Data are presented as mean±SEM. (***: P<0.001, relative to last day of cocaine retraining; †††: P<0.001, relative to last day of extinction; ###: P<0.001, relative to Saline-Yohimbine 2 mg/kg treatment). N=15-17 in each treatment group.

FIG. 15 shows acquisition of oxycodone self-administration. A stable oxycodone self-administration was achieved in 20 days of acquisition training. Data are presented as means±SEM.

FIG. 16 shows effects of Compound 1 (0.3, 1 & 3 mg/kg), and MTEP (3 mg/kg) on oxycodone self-administration in rats. Data are presented as mean±SEM. *: P<0.05, **: P<0.01 and ***: P<0.001 compared to vehicle.

FIG. 17 shows the effects of extinction training and Compound 1 (3 mg/kg) & MTEP (3 mg/kg) on the reinstatement of oxycodone self-administration in rats. Data are presented as mean±SEM. *: P<0.05, **: P<0.01 and ***: P<0.001.

### DETAILED DESCRIPTION

Substance use disorder (SUD) treatment can be divided into three phases: (1) from withdrawal to abstinence, whereby the purpose is to lead the patient to discontinue consumption of the substance; (2) abstinence and relapse prevention; and (3) reduction of substance consumption. For example, alcohol use disorder (AUD) treatment can be divided into three phases: (1) from withdrawal to abstinence, whereby the purpose is to lead the patient to discontinue alcohol consumption; (2) abstinence and relapse prevention; and (3) reduction of alcohol consumption. See, for example, Guglielmo, R et al. “Pharmacological treatments in alcohol use disorders: state of art and new perspectives.” La Clinica terapeutica vol. 166,6 (2015): 262-70, the contents of which are herein incorporated by reference. Different treatments and indeed different drugs are employed in these phases.

The present disclosure relates to abstinence and relapse prevention, and in particular to methods for treating subjects having an SUD (e.g., AUD, OUD, and/or a stimulant use disorder such as CUD) with 4-(3-cyanophenyl)-6-pyridinylpyrimidine mGlu5 negative allosteric modulators (e.g., Compound 1 or Compound 2). It may be that treating a subject having an SUD who is at risk of relapse to substance use reduces susceptibility to one or more relapse triggers. It may also be that treating a subject having an SUD with 4-(3-cyanophenyl)-6-pyridinylpyrimidine mGlu5 negative allosteric modulators (e.g., Compound 1 or Compound 2) promotes remission in the subject.

The following description sets forth numerous exemplary configurations, methods, parameters, and the like. It should be recognized, however, that such description is not intended as a limitation on the scope of the present disclosure but is instead provided as a description of exemplary embodiments.

As used herein, the terms “including,” “containing,” and “comprising” are used in their open, non-limiting sense.

The articles “a” and “an”, as used herein, refer to one or more than one (i.e., to at least one) of the grammatical object of the article. By way of example, “an embodiment” refers to one element or more than one embodiment.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

The present disclosure provides methods of treating a subject having an SUD (e.g., AUD, OUD, and/or a stimulant use disorder such as CUD) with a compound (e.g., Compound 1 or Compound 2), such as a subject at risk of relapse to substance use and/or a subject in remission in need of help to maintain remission. In some embodiments, the methods reduce substance intake of the subject. In some embodiments, the compound is Compound 1.

Accordingly, the present disclosure also provides methods of treating a subject having alcohol use disorder (AUD) with a 4-(3-cyanophenyl)-6-pyridinylpyrimidine derivative (e.g., Compound 1 or Compound 2), such as a subject at risk of relapse to alcohol use and/or a subject in remission in need of help to maintain remission. In some embodiments, the methods reduce alcohol intake of the subject. In some embodiments, the compound is Compound 1.

The present disclosure also provides methods of treating a subject having opioid use disorder (OUD) with a 4-(3-cyanophenyl)-6-pyridinylpyrimidine derivative (e.g., Compound 1 or Compound 2), such as a subject at risk of relapse to opioid use and/or a subject in remission in need of help to maintain remission. In some embodiments, the methods reduce opioid intake of the subject. In some embodiments, the compound is Compound 1.

The present disclosure also provides methods of treating a subject having a stimulant (e.g., cocaine) use disorder with a 4-(3-cyanophenyl)-6-pyridinylpyrimidine derivative (e.g., Compound 1 or Compound 2), such as a subject at risk of relapse to stimulant use and/or a subject in remission in need of help to maintain remission. In some embodiments, the methods reduce stimulant (e.g., cocaine) intake of the subject. In some embodiments, the stimulant use disorder is cocaine use disorder (CUD). In some embodiments, the compound is Compound 1.

The present disclosure also provides methods of treating a subject having 2, 3, 4, or more SUDs with a 4-(3-cyanophenyl)-6-pyridinylpyrimidine derivative (e.g., Compound 1 or Compound 2), such as a subject at risk of relapse to 2, 3, 4, or more substances use and/or a subject in remission in need of help to maintain remission. In some embodiments, the methods reduce 2, 3, 4, or more substances intake of the subject. In some embodiments, the subject has AUD and OUD. In some embodiments, the subject has AUD and a stimulant use disorder such as CUD. In some embodiments, the subject has OUD and a stimulant use disorder such as CUD. In some embodiments, the compound is Compound 1.

The present disclosure also provides methods of treating a subject having an SUD (e.g., AUD, OUD, and/or a stimulant use disorder such as CUD) who is at risk of relapse to substance use, the methods comprising administering to the subject a therapeutically effective amount of a 4-(3-cyanophenyl)-6-pyridinylpyrimidine derivative (e.g., Compound 1 or Compound 2) of the formula

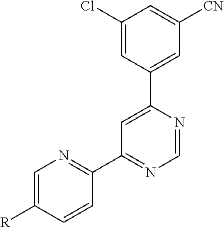

or a pharmaceutically acceptable salt thereof, wherein R is H, Me, F, Cl or cyano, wherein the administration reduces susceptibility to one or more relapse triggers. In some embodiments, the administration results in remission of the SUD (e.g., AUD, OUD, and/or a stimulant use disorder such as CUD). In some embodiments, the administration results in remission for at least 3 months. In some embodiments, the administration results in remission for at least 12 months. In some embodiments, the SUD is AUD. In some embodiments, the compound is Compound 1.

The present disclosure also provides methods of promoting remission in a subject having an SUD (e.g., AUD, OUD, and/or a stimulant use disorder such as CUD), the method comprising administering to the subject a therapeutically effective amount of a 4-(3-cyanophenyl)-6-pyridinylpyrimidine derivative of the formula

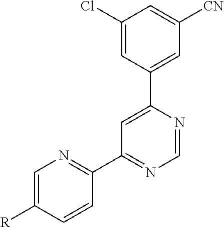

or a pharmaceutically acceptable salt thereof, wherein R is H, Me, F, Cl or cyano. In some embodiments, the administration results in remission for at least 3 months. In some embodiments, the administration results in remission for at least 12 months. In some embodiments, the SUD is AUD. In some embodiments, the compound is Compound 1.

The terms “administered”, “administration”, or “administering”, as used herein, refers to either directly administering a 4-(3-cyanophenyl)-6-pyridinylpyrimidine derivative (e.g., Compound 1 or Compound 2) or pharmaceutically acceptable salt thereof or a composition thereof to a subject in need of treatment by bringing such individual in contact with, or otherwise exposing such individual to, such compound.

As used herein, the term “subject” encompasses mammals and non-mammals. Examples of mammals include, but are not limited to, any member of the class Mammalia: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In some embodiments, the subject is a human. The term “patient” is used interchangeably herein with the term “subject” when the subject is human.

A substance use disorder (SUD), as disclosed herein, refers to a substance related disorder (SRD) that is a use disorder. Examples of SUDs are alcohol use disorder (AUD), opioid use disorder (OUD), and stimulant use disorder. Stimulants include amphetamine-type substances and cocaine. An example stimulant use disorder is cocaine use disorder (CUD).

As used herein, AUD, OUD, and stimulant use disorders such as CUD refer to a mental disorder which may be diagnosed by trained medical professionals in accordance with diagnostic criteria presented in Diagnostic and Statistical Manual of Mental Disorders (DSM-5), the contents of which are herein incorporated by reference.

SUDs (e.g., AUD, OUD, and stimulant use disorder such as CUD) refer to a problematic pattern of substance use leading to clinically significant impairment or distress, as manifested by at least two of the following SUD symptoms (e.g, AUD symptoms, OUD symptoms, or a stimulant use disorder symptoms such as CUD symptoms) occurring within a 12-month period:

1. A substance (e.g., alcohol, an opioid, or a stimulant such as cocaine) is often taken in larger amounts or over a longer period than was intended.

2. There is a persistent desire or unsuccessful efforts to cut down or control substance (e.g., alcohol, an opioid, or a stimulant such as cocaine) use.

3. A great deal of time is spent in activities necessary to obtain the substance (e.g., alcohol, an opioid, or a stimulant such as cocaine), use the substance (e.g., alcohol, an opioid, or a stimulant such as cocaine), or recover from its effects.

4. Craving, or a strong desire or urge to use the substance (e.g., alcohol, an opioid, or a stimulant such as cocaine).

5. Recurrent substance (e.g., alcohol, an opioid, or a stimulant such as cocaine) use resulting in a failure to fulfill major role obligations at work, school, or home.

6. Continued substance (e.g., alcohol, an opioid, or a stimulant such as cocaine) use despite having persistent or recurrent social or interpersonal problems caused or exacerbated by the effects of the substance (e.g., alcohol, an opioid, or a stimulant such as cocaine).

7. Important social, occupational, or recreational activities are given up or reduced because of substance (e.g., alcohol, an opioid, or a stimulant such as cocaine) use.

8. Recurrent substance (e.g., alcohol, an opioid, or a stimulant such as cocaine) use in situations in which it is physically hazardous.

9. Substance (e.g., alcohol, an opioid, or a stimulant such as cocaine) use is continued despite knowledge of having a persistent or recurrent physical or psychological problem that is likely to have been caused or exacerbated by alcohol.

10. Tolerance, as defined by either of the following: (a) A need for markedly increased amounts of the substance to achieve intoxication or desired effect. (b) A markedly diminished effect with continued use of the same amount of the substance.

11. Withdrawal, as manifested by either of the following: (a) Withdrawal syndrome for the substance. (b) The substance (or a closely related substance, such as a benzodiazepine for alcohol) is taken to relieve or avoid withdrawal symptoms.

Some embodiments further comprise determining if the subject has 1, 2, 3 or more SUD (e.g., AUD, OUD, and/or a stimulant use disorder such as CUD) symptoms. Some embodiments further comprise treating the subject if the subject has 1, 2, 3 or more SUD (e.g., AUD, OUD, and/or a stimulant use disorder such as CUD) symptoms. Some embodiments further comprise determining if the subject has 1 or more SUD (e.g., AUD, OUD, and/or a stimulant use disorder such as CUD) symptoms. Some embodiments further comprise treating the subject if the subject has 1 or more SUD (e.g., AUD, OUD, and/or a stimulant use disorder such as CUD) symptoms. Some embodiments further comprise determining if the subject has 2 or more SUD (e.g., AUD, OUD, and/or a stimulant use disorder such as CUD) symptoms. Some embodiments further comprise treating the subject if the subject has 2 or more SUD (e.g., AUD, OUD, and/or a stimulant use disorder such as CUD) symptoms.

Some embodiments further comprise reducing one or more SUD (e.g., AUD, OUD, and/or a stimulant use disorder such as CUD) symptoms. With reference to the SUD (e.g., AUD, OUD, and/or a stimulant use disorder such as CUD) symptom list above, some embodiments reduce symptom 1, 2, 3, 4, 5, 6, 7, 8, 9, 10, or 11 or any combination thereof. Some embodiments reduce symptom 1, 2, 4, or 5, or any combination thereof. Some embodiments reduce symptom 1. Some embodiments reduce symptom 2. Some embodiments reduce symptom 4. Some embodiments reduce symptom 5. Some embodiments reduce substance (e.g., alcohol, opioid, or a stimulant such as cocaine) intake in a subject. Some embodiments reduce alcohol intake in a subject having an SUD (e.g., AUD, OUD, and/or a stimulant use disorder such as CUD). In some embodiments, the methods reduce substance (e.g., alcohol, opioid, or a stimulant such as cocaine) intake in a subject having an SUD (e.g., AUD, OUD, and/or a stimulant use disorder such as CUD) who is at risk of relapse to substance (e.g., alcohol, opioid, or a stimulant such as cocaine) use. Some embodiments reduce withdrawal symptoms in a subject having AUD.

The SUD (e.g., AUD, OUD, and/or a stimulant use disorder such as CUD) may be categorized as Mild SUD (presence of 2-3 symptoms; e.g., Mild AUD, Mild OUD, or a mild stimulant use disorder such as Mild CUD), Moderate SUD (presence of 4-5 symptoms; e.g., Moderate AUD, Moderate OUD, or a moderate stimulant use disorder such as Moderate CUD), or Severe SUD (presence of 6 or more symptoms; e.g., Severe AUD, Severe OUD, or a severe stimulant use disorder such as Severe CUD). Some embodiments comprise determining if the subject has at least Mild SUD (e.g., Mild AUD, Mild OUD, or a mild stimulant use disorder such as Mild CUD). Some embodiments comprise treating a subject with Mild SUD (e.g., Mild AUD, Mild OUD, or a mild stimulant use disorder such as Mild CUD). Some embodiments comprise determining if the subject has at least Moderate SUD (e.g., Moderate AUD, Moderate OUD, or a moderate stimulant use disorder such as Moderate CUD). Some embodiments comprise treating a subject with Moderate SUD (e.g., Moderate AUD, Moderate OUD, or a moderate stimulant use disorder such as Moderate CUD). Some embodiments further comprise determining if the subject has severe SUD (e.g., Severe AUD, Severe OUD, or a severe stimulant use disorder such as Severe CUD). Some embodiments comprise treating a subject with Severe SUD (e.g., Severe AUD, Severe OUD, or a severe stimulant use disorder such as Severe CUD). Some embodiments reduce presence of one or more SUD e.g., AUD, OUD, and/or a stimulant use disorder such as CUD). Some embodiments reduce the severity of the SUD (e.g., reduce Severe SUD to Mild SUD or reduce Moderate SUD to Mild SUD).

In some embodiments, the method further comprises determining if the subject meets the DSM-5 diagnostic criteria of an SUD (e.g., AUD, OUD, and/or a stimulant use disorder such as CUD). In some embodiments, the method further comprises determining if the subject meets the DSM-5 diagnostic criteria of an SUD (e.g., AUD, OUD, and/or a stimulant use disorder such as CUD), administering the 4-(3-cyanophenyl)-6-pyridinylpyrimidine derivative (e.g., Compound 1 or Compound 2) or a pharmaceutically acceptable salt thereof if the subject meets the DSM-5 criteria of the SUD (e.g., AUD, OUD, and/or a stimulant use disorder such as CUD), and not administering the 4-(3-cyanophenyl)-6-pyridinylpyrimidine derivative (e.g., Compound 1 or Compound 2) or a pharmaceutically acceptable salt thereof if the subject does not meet the DSM-5 diagnostic criteria of the SUD (e.g., AUD, OUD, and/or a stimulant use disorder such as CUD).

A subject who develops an SUD (e.g., AUD, OUD, and/or a stimulant use disorder such as CUD) and later loses some or most symptoms is not necessarily cured of the SUD (e.g., AUD, OUD, and/or a stimulant use disorder such as CUD). Rather, an SUD (e.g., AUD, OUD, and/or a stimulant use disorder such as CUD) often persists in subjects with the SUD (e.g., AUD, OUD, and/or a stimulant use disorder such as CUD) in a state referred to as remission.

Remission, as used herein, refers to a state in a subject where, after full criteria for an SUD (e.g., AUD, OUD, and/or a stimulant use disorder such as CUD) was previously met, none of the criteria for the SUD (e.g., AUD, OUD, and/or a stimulant use disorder such as CUD) are met (with the exception that Criterion A4, “Craving, or a strong desire or urge to use the substance,” may be met). Early remission refers to remission for at least 3 months but for less than 12 months. Sustained remission refers to remission for at least 12 months. As used herein, a subject in remission is understood to still be a subject having an SUD (e.g., AUD, OUD, and/or a stimulant use disorder such as CUD).

Abstinence, as used herein, refers to a period in which a subject does not consume the substance (e.g., alcohol, opioids, and/or stimulants such as cocaine). Examples of tests to confirm abstinence include self-assessment tests and laboratory tests (e.g., Timeline-Followback (TFLB) self-reported substance consumption assessment, acute alcohol ingestion tests, and chronic alcohol tests, which are discussed in more detail below).

In some embodiments, the subject is abstinent. In some embodiments, the subject is already abstinent and is at risk of relapse. In some of these embodiments, the method further comprises administering the 4-(3-cyanophenyl)-6-pyridinylpyrimidine derivative (e.g., Compound 1 or Compound 2) or a pharmaceutically acceptable salt thereof to the subject after a period of abstinence by the subject. In some embodiments, method further comprises performing a test to confirm abstinence.

In some embodiments, the period of abstinence is at least 12 hours and the subject has a breath or blood alcohol level of about 0. In some embodiments, the period of abstinence is at least 1 day, at least 3 days, at least 5 days, at least 1 week, at least 2 weeks, at least 3 weeks, at least 1 month, at least 3 months, at least 6 months, or at least 12 months. In some embodiments, the period of abstinence is at least 1 month. In some embodiments, the period of abstinence is at least 3 months. In some embodiments, the period of abstinence is at least 6 months. In some embodiments, the period of abstinence is at least 12 months.

Relapse, as used herein, refers to resumption of substance (e.g., alcohol, opioids, and/or stimulants such as cocaine) use following a prolonged period of abstinence. Clinically, vulnerability to relapse is commonly associated with an intense craving or desire to use a substance. Relapse represents a prevalent and significant problem in an SUD (e.g., AUD, OUD, and/or a stimulant use disorder such as CUD). In fact, given the high rate of recidivism in SUD (e.g., AUD, OUD, and/or a stimulant use disorder such as CUD), relapse clearly is a major impediment to treatment efforts. For example, clinical laboratory studies have found that compared with control subjects, alcohol-dependent people are more sensitive to these relapse triggers, which in turn presumably drives an increased desire to drink.

In some embodiments, administration of a 4-(3-cyanophenyl)-6-pyridinylpyrimidine derivative (e.g., Compound 1 or Compound 2) or pharmaceutically acceptable salt thereof prevents relapse for at least 3 months. In some embodiments, administration of a 4-(3-cyanophenyl)-6-pyridinylpyrimidine derivative (e.g., Compound 1 or Compound 2) or pharmaceutically acceptable salt thereof prevents relapse for at least 12 months.

Some embodiments include measuring substance (e.g., alcohol, opioids, and/or stimulants such as cocaine) use using one or more tests. In some embodiments, the methods reduce substance (e.g., alcohol, opioids, and/or stimulants such as cocaine) intake of the subject as measured by one or more tests (e.g., self-assessment tests and laboratory tests). In some embodiments, the methods include one or more tests to determine relapse. Tests for relapse include self-assessment tests and laboratory tests.

A Timeline-Followback (TFLB) self-reported substance consumption assessment is a retrospective report of daily substance (e.g., alcohol, opioids, and/or stimulants such as cocaine) consumption over the past 30 days and is commonly used to assess relapse. TFLB is described in Sobell L, Sobell M. Timeline Followback: A Technique for Assessing Self Reported Ethanol Consumption. Vol. 17. Totowa, NJ: Humana Press, and Sobell L C, Sobell M (1996). Timeline Followback Method (Drugs, Cigarettes, and Marijuana), the contents of which are herein incorporated by reference. In some embodiments, one of one or more tests to determine relapse is a Timeline-Followback self-reported substance (e.g., alcohol, opioids, and/or stimulants such as cocaine) consumption assessment. Some embodiments include measuring substance (e.g., alcohol, opioids, and/or stimulants such as cocaine) use with a TFLB self-reported substance consumption assessment. In some embodiments, the methods reduce substance (e.g., alcohol, opioids, and/or stimulants such as cocaine) intake of the subject as measured by a TFLB self-reported substance consumption assessment.

Laboratory tests for relapse include urine drug tests, saliva drug tests (also known as oral fluid tests), and hair follicle drug tests. For opioids and stimulants (e.g., cocaine), the most common test used in the clinic is the urine drug screening test.

Urine Drug Tests: Urine drug tests are the most commonly used method for detecting substance use, especially for opioids and stimulants (e.g., cocaine). To perform a urine drug test, a urine sample is collected from the individual in a sterile container. The sample is then analyzed using immunoassay techniques, which detect the presence of drug metabolites. If the initial test yields positive results, it is usually followed by a confirmatory test using gas chromatography-mass spectrometry (GC-MS) or liquid chromatography-tandem mass spectrometry (LC-MS/MS) to ensure accuracy. Detection times for substances can vary. For example, opioids and cocaine can be detected in urine for 1-3 days, while ethanol (alcohol) can be detected for 12-24 hours after use. Additional details may be found in the following reference, herein incorporated by reference Center for Substance Abuse Treatment. Substance Abuse: Clinical Issues in Intensive Outpatient Treatment. Rockville (MD): Substance Abuse and Mental Health Services Administration (US); 2006. (Treatment Improvement Protocol (TIP) Series, No. 47.) Appendix B. Urine Collection and Testing Procedures and Alternative Methods for Monitoring Drug Use. (https://www.ncbi.nlm.nih.gov/books/NBK64092/

Saliva Drug Tests (Oral Fluid Tests): Saliva drug tests are a non-invasive method for detecting drug use. To perform this test, a swab is used to collect saliva from the individual's mouth, typically from the inner cheek or under the tongue. The saliva sample is then analyzed for the presence of drugs using immunoassay techniques. Like urine tests, positive results are usually confirmed with GC-MS or LC-MS/MS. Detection times for substances in saliva can vary. Opioids can be detected for up to 36 hours, while cocaine can be detected for 24-48 hours after use. Additional details may be found in the following reference, herein incorporated by reference: Cone, E. J., & Huestis, M. A. (2007). Interpretation of Oral Fluid Tests for Drugs of Abuse. Annals of the New York Academy of Sciences, 1098 (1), 51-103. https://doi.org/10.1196/annals.1384.037.

Hair Follicle Drug Tests: Hair follicle drug tests involve collecting a small sample of hair, usually from the head. This test provides a longer detection window compared to urine or saliva tests. Hair samples are analyzed using techniques like enzyme-linked immunosorbent assay (ELISA) for screening and GC-MS or LC-MS/MS for confirmation. Hair drug tests can detect drug use over a period of approximately 90 days, depending on the length of the hair sample. Additional details may be found in the following reference, herein incorporated by reference: Gryczynski J, Schwartz R P, Mitchell S G, O'Grady K E, Ondersma S J. Hair drug testing results and self-reported drug use among primary care patients with moderate-risk illicit drug use. Drug Alcohol Depend. 2014 Aug. 1; 141:44-50. https://doi: 10.1016/j.drugalcdep.2014.05.001.

The detection times and ranges can vary depending on factors such as individual metabolism, frequency of drug use, and the sensitivity of the testing method.

Laboratory tests for AUD relapse include acute alcohol ingestion tests and chronic alcohol use tests. Examples tests are described in Nanau R M, Neuman M G., Biomolecules and biomarkers used in diagnosis of alcohol drinking and in monitoring therapeutic interventions, Biomolecules, 2015; 5 (3): 1339-1385), the contents of which are herein incorporated by reference. In some embodiments, the methods reduce alcohol intake of the subject as measured by an acute alcohol ingestion test. In some embodiments, the methods reduce alcohol intake of the subject as measured by a chronic alcohol use test.

Acute alcohol ingestion tests include laboratory tests for ethanol (e.g., breath or blood alcohol tests), ethyl glucuronide (EtG), and ethyl sulfate (EtS). EtG and EtS are direct minor metabolites of ethanol and are considered good markers of acute, short-term (up to 36 hours in the blood, up to 5 days in urine) alcohol ingestion. The sensitivity of these tests is highest in heavy drinkers but wanes after 24 hours and with lower doses. Results do not accurately correlate with the amount or frequency of ethanol use. Because false-positive results do occur with the screening test, it is strongly recommended that all positive screening results be confirmed with an EtG/EtS confirmatory test. There are no false positive results with the confirmatory test. False-negative results are extremely rare with the EtG screening test. EtG and EtS results are legally defensible. The 2012 SAMSHA Advisory includes the following preliminary guidance on what positive results may indicate: >1,000 ng/ml: Heavy drinking on the same day or previously (e.g., previous day or two), or light drinking the same day; 500-1,000 ng/ml: Previous heavy drinking (previous 1-3 days), Recent light drinking (e.g., past 24 hours), or Recent intense “extraneous exposure” (within 24 hours or less); 100-500 ng/ml: Previous heavy drinking (1-3 days), Previous light drinking (12-36 hours), or Recent “extraneous” exposure. Some embodiments include measuring alcohol use with an acute alcohol ingestion test. In some embodiments, the methods reduce alcohol intake of the subject as measured by an acute alcohol ingestion test. In some embodiments, one of one or more tests to determine relapse is an acute alcohol ingestion test.

Chronic alcohol tests include tests for carbohydrate-deficient transferrin (CDT) and phosphatidylethanol (PEth), which are useful markers for monitoring abstinence and relapse after long-term use. Some embodiments include measuring alcohol use with a chronic alcohol test. In some embodiments, the methods reduce alcohol intake of the subject as measured by a chronic alcohol test. In some embodiments, one of one or more tests to determine relapse is a chronic alcohol use test.

Carbohydrate-Deficient Transferrin: CDT, an indirect metabolite of ethanol, is a serum marker of long-term, heavy alcohol use (≥40 g/day for up to 2 weeks) or relapse. CDT concentrations generally correlate well with an individual's drinking pattern, especially during the preceding 30 days, and are most useful for long-term abstinence monitoring and relapse detection. Factors that affect CDT levels include body mass index (BMI), female sex, and smoking. CDT testing cannot be used in individuals suspected of having congenital glycosylation disorders.

Phosphatidylethanol: PEth is a direct ethanol metabolite and can be tested to detect longer-term exposure (within 1-2 weeks or longer). Because blood PEth levels are closely correlated with alcohol consumption, PEth testing can be used to monitor alcohol consumption, identify early signs of harmful alcohol consumption, and track cases of AUD or dependence.

Relapse triggers, as used herein, refers to cravings for a substance (e.g., alcohol, opioids, and/or stimulants such as cocaine) and to urges associated with cues. Cravings and cues refer to separate concepts, though are often associated with each other. Cues may induce cravings, though cravings may occur independent of cues. Cues also may also lead to substance (e.g., alcohol, opioids, and/or stimulants such as cocaine) use without necessarily inducing cravings.

Cravings, as used herein, refers to an intense desire or urge for a substance (e.g., alcohol, opioids, and/or stimulants such as cocaine) that may occur at any time but is more likely when in an environment where the substance (e.g., alcohol, opioids, and/or stimulants such as cocaine) previously was obtained or used. Craving has also been shown to involve classical conditioning and is associated with the activation of specific reward structures in the brain. Subject's cravings are queried by asking if there has ever been a time when they had such strong urges to consume the substance (e.g., alcohol, opioids, and/or stimulants such as cocaine) that they could not think of anything else.

Cues, as used herein, refer to social, environmental, or emotional situations that remind people in recovery of their past substance (e.g., alcohol, opioids, and/or stimulants such as cocaine) use. These cues bring about urges that may lead to a relapse. While cues do not force a person to use the substance, they increase the likelihood of use. Examples of cues include internal cues, such as sadness, anxiety or stress, anger or irritation, physical pain or discomfort and external cures, such as smelling the substance, having financial problems, visiting a bar or restaurant where the patient used to use the substance, attending a party, seeing the substance (e.g., seeing alcoholic beverages, interacting with people who use, and experiencing emotional or physical abuse.

Relapse can be a gradual process with distinct stages. It can begin weeks and sometime months before an individual consumes the substance (e.g., alcohol, opioids, and/or stimulants such as cocaine). A goal of treatment is to help individuals recognize and control the early stages, in which the chances of success are greatest. As used herein, symptoms associated with the early stages of relapse are referred to as early-relapse symptoms. Early-relapse symptoms can include emotional and mental symptoms, as described in Melemis S M. Relapse Prevention and the Five Rules of Recovery. Yale J Biol Med. 2015 Sep. 3; 88 (3): 325-32, herein incorporated by reference. In some embodiments, administration of a 4-(3-cyanophenyl)-6-pyridinylpyrimidine derivative (e.g., Compound 1 or Compound 2) or pharmaceutically acceptable salt thereof results in the subject having no early-relapse symptoms for at least 3 months. In some embodiments, administration of a 4-(3-cyanophenyl)-6-pyridinylpyrimidine derivative (e.g., Compound 1 or Compound 2) or pharmaceutically acceptable salt thereof results in the subject having no early-relapse symptoms for at least 12 months.

Emotional early-relapse symptoms include: (1) irritability or moodiness. Sometimes, a shift in mood can happen because of an emotional trigger or guilt over wanting to use again; (2) isolating; (3) not going to meetings/medical appointments; (4) going to meetings/medical appointments but not sharing; (5) focusing on others (focusing on other people's problems or focusing on how other people affect them); and (6) poor eating and sleeping habits. The common denominator of emotional relapse symptoms is poor self-care, in which self-care is broadly defined to include emotional, psychological, and physical care.

Mental early-relapse symptoms include: (1) craving for the substance (e.g., alcohol, opioids, and/or stimulants such as cocaine); (2) thinking about people, places, and things associated with past use; (3) minimizing consequences of past use or glamorizing past use; (4) bargaining (bargaining, individuals start to think of scenarios in which it would be acceptable to use); (5) lying; (6) thinking of schemes to better control using; (7) looking for relapse opportunities; (8) planning a relapse.

### Compounds

Embodiments of the present disclosure are directed methods involving compounds of the formula

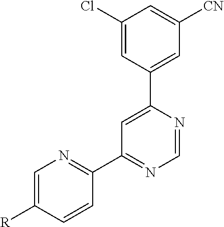

or pharmaceutically acceptable salts thereof, wherein R is H, Me, F, Cl or cyano. In some embodiments, R is F or cyano. In some embodiments, R is F. In some embodiments, R is cyano. The compound (e.g., Compound 1 or Compound 2) may sometimes be referred to herein as a 4-(3-cyanophenyl)-6-pyridinylpyrimidine derivative, however, unless explicitly stated, said term is not meant to limit the compound to the product of any particular synthetic route. The compound (e.g., Compound 1 or Compound 2) may also sometimes be referred to herein as a 4-(3-cyanophenyl)-6-pyridinylpyrimidine mGlu5 negative allosteric modulator, however, unless explicitly stated, said term is not meant to limit the compound to one associated with a particular mechanism of action or one shown by any particular assay or other test as acting on a particular target or binding site. Rather, these terms provide supplemental context and should not be read as putting additional limitations on the compounds described herein, which are compounds of the given formula (e.g., Compound 1 or Compound 2), or a pharmaceutically acceptable salt thereof.

In some embodiments, the 4-(3-cyanophenyl)-6-pyridinylpyrimidine derivative is Compound 1,

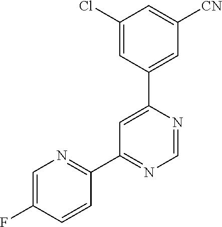 [SMILES: N#Cc1cc(Cl)cc(-c2cc(-c3ccc(F)cn3)ncn2)c1]

or a pharmaceutically acceptable salt thereof.

In some embodiments, the 4-(3-cyanophenyl)-6-pyridinylpyrimidine derivative is Compound 2,

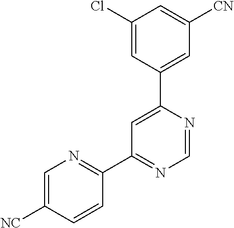 [SMILES: N#Cc1ccc(-c2cc(-c3cc(Cl)cc(C#N)c3)ncn2)nc1]

or a pharmaceutically acceptable salt thereof.

“Pharmaceutically acceptable”, as used herein, refers to that which is useful in preparing a pharmaceutical composition that is generally safe, non-toxic and not biologically or otherwise undesirable, and includes that which is acceptable for veterinary use as well as human pharmaceutical use. For example, some embodiments employ a pharmaceutical composition comprising Compound 1 or Compound 2, or a pharmaceutically acceptable salt thereof, and a pharmaceutically acceptable excipient.

“Pharmaceutically acceptable salt”, as used herein, refers to a salt which is generally safe, non-toxic and not biologically or otherwise undesirable, and includes that which is acceptable for veterinary use as well as human pharmaceutical use. Some embodiments employ a pharmaceutically acceptable salt of Compound 1. Some embodiments employ a pharmaceutically acceptable salt of Compound 2.

The terms “effective amount” or “therapeutically effective amount”, when used in connection with a compound, refers to a sufficient amount of the compound to provide the desired biological result. That result can be reduction and/or alleviation of the signs, symptoms, or causes of an SUD (e.g., AUD, OUD, and/or a stimulant use disorder such as CUD), or any other desired alteration of a biological system. For example, an “effective amount” for therapeutic use is the amount of the composition comprising a compound as disclosed herein required to provide a clinically significant decrease in a sign, symptom, or cause of relapse to substance (e.g., alcohol, opioids, and/or stimulants such as cocaine) use. Thus, the expression “effective amount” generally refers to the quantity for which the active substance has therapeutic effects.

### Dosage

Dosages of a 4-(3-cyanophenyl)-6-pyridinylpyrimidine derivative (e.g., Compound 1 or Compound 2) or a pharmaceutically acceptable salt thereof may be efficacious in the treatment of subjects having an SUD (e.g., AUD, OUD, and/or a stimulant use disorder such as CUD) as described herein. In some embodiments, the dosage is administered over multiple daily administrations (e.g., taken multiple times over the course of a day). In some embodiments, a 4-(3-cyanophenyl)-6-pyridinylpyrimidine derivative (e.g., Compound 1 or Compound 2) or a pharmaceutically acceptable salt thereof is administered twice-daily, thrice-daily, or quadrice-daily. The sum of the 4-(3-cyanophenyl)-6-pyridinylpyrimidine derivative (e.g., Compound 1 or Compound 2) or pharmaceutically acceptable salt thereof administered over the multiple daily administrations is the dosage.

In some embodiments, the subject is administered a dosage of about 5 mg/day to about 300 mg/day, about 10 mg/day to about 300 mg/day, about 20 mg/day to about 300 mg/day, about 30 mg/day to about 300 mg/day, about 40 mg/day to about 300 mg/day, about 50 mg/day to about 300 mg/day, about 60 mg/day to about 300 mg/day, about 70 mg/day to about 300 mg/day, about 5 mg/day to about 200 mg/day, about 10 mg/day to about 200 mg/day, about 20 mg/day to about 200 mg/day, about 30 mg/day to about 200 mg/day, about 40 mg/day to about 200 mg/day, about 50 mg/day to about 200 mg/day, about 60 mg/day to about 200 mg/day, about 70 mg/day to about 200 mg/day, about 5 mg/day to about 120 mg/day, about 10 mg/day to about 120 mg/day, about 20 mg/day to about 120 mg/day, about 30 mg/day to about 120 mg/day, about 40 mg/day to about 120 mg/day, about 50 mg/day to about 120 mg/day, about 60 mg/day to about 120 mg/day, about 70 mg/day to about 120 mg/day, about 5 mg/day to about 100 mg/day, about 10 mg/day to about 100 mg/day, about 20 mg/day to about 100 mg/day, about 30 mg/day to about 100 mg/day, about 40 mg/day to about 100 mg/day, about 50 mg/day to about 100 mg/day, about 60 mg/day to about 100 mg/day, about 70 mg/day to about 100 mg/day, about 5 mg/day to about 90 mg/day, about 10 mg/day to about 90 mg/day, about 20 mg/day to about 90 mg/day, about 30 mg/day to about 90 mg/day, about 40 mg/day to about 90 mg/day, about 50 mg/day to about 90 mg/day, about 60 mg/day to about 90 mg/day, about 70 mg/day to about 90 mg/day, about 5 mg/day to about 80 mg/day, about 10 mg/day to about 80 mg/day, about 20 mg/day to about 80 mg/day, about 30 mg/day to about 80 mg/day, about 40 mg/day to about 80 mg/day, about 50 mg/day to about 80 mg/day, about 60 mg/day to about 80 mg/day, about 70 mg/day to about 80 mg/day, about 5 mg/day to about 70 mg/day, about 10 mg/day to about 70 mg/day, about 20 mg/day to about 70 mg/day, about 30 mg/day to about 70 mg/day, about 40 mg/day to about 70 mg/day, about 50 mg/day to about 70 mg/day, or about 60 mg/day to about 70 mg/day of the 4-(3-cyanophenyl)-6-pyridinylpyrimidine derivative (e.g., Compound 1 or Compound 2) or a pharmaceutically acceptable salt thereof.

In some embodiments, the subject is administered a dosage of about 5 mg/day, about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 200 mg/day, or about 300 mg/day of the 4-(3-cyanophenyl)-6-pyridinylpyrimidine derivative (e.g., Compound 1 or Compound 2) or a pharmaceutically acceptable salt thereof.

It was surprisingly found that subjects administered 100 mg/day of Compound 1 had unexpectedly high exposure of the compound. Accordingly, in some embodiments, the subject is administered a dosage below about 100 mg/day. In some embodiments, the subject is administered about 5 mg/day to about 100 mg/day, about 10 mg/day to about 100 mg/day, about 20 mg/day to about 100 mg/day, about 30 mg/day to about 100 mg/day, about 40 mg/day to about 100 mg/day, about 50 mg/day to about 100 mg/day, about 60 mg/day to about 100 mg/day, about 70 mg/day to about 100 mg/day, about 5 mg/day to about 90 mg/day, about 10 mg/day to about 90 mg/day, about 20 mg/day to about 90 mg/day, about 30 mg/day to about 90 mg/day, about 40 mg/day to about 90 mg/day, about 50 mg/day to about 90 mg/day, about 60 mg/day to about 90 mg/day, about 70 mg/day to about 90 mg/day, about 5 mg/day to about 80 mg/day, about 10 mg/day to about 80 mg/day, about 20 mg/day to about 80 mg/day, about 30 mg/day to about 80 mg/day, about 40 mg/day to about 80 mg/day, about 50 mg/day to about 80 mg/day, about 60 mg/day to about 80 mg/day, about 70 mg/day to about 80 mg/day, about 5 mg/day to about 70 mg/day, about 10 mg/day to about 70 mg/day, about 20 mg/day to about 70 mg/day, about 30 mg/day to about 70 mg/day, about 40 mg/day to about 70 mg/day, about 50 mg/day to about 70 mg/day, or about 60 mg/day to about 70 mg/day of the 4-(3-cyanophenyl)-6-pyridinylpyrimidine derivative (e.g., Compound 1 or Compound 2) or a pharmaceutically acceptable salt thereof. In some embodiments, the subject is administered a dosage of about 5 mg/day to about 100 mg/day of the 4-(3-cyanophenyl)-6-pyridinylpyrimidine derivative (e.g., Compound 1 or Compound 2) or a pharmaceutically acceptable salt thereof. Some embodiments are directed to a method of treating a subject having an SUD (e.g., AUD, OUD, and/or a stimulant use disorder such as SUD), wherein the method comprises administering a dosage of about 5 mg/day to about 100 mg/day, about 10 mg/day to about 100 mg/day, about 20 mg/day to about 100 mg/day, about 30 mg/day to about 100 mg/day, about 40 mg/day to about 100 mg/day, about 50 mg/day to about 100 mg/day, about 60 mg/day to about 100 mg/day, about 70 mg/day to about 100 mg/day, about 5 mg/day to about 90 mg/day, about 10 mg/day to about 90 mg/day, about 20 mg/day to about 90 mg/day, about 30 mg/day to about 90 mg/day, about 40 mg/day to about 90 mg/day, about 50 mg/day to about 90 mg/day, about 60 mg/day to about 90 mg/day, about 70 mg/day to about 90 mg/day, about 5 mg/day to about 80 mg/day, about 10 mg/day to about 80 mg/day, about 20 mg/day to about 80 mg/day, about 30 mg/day to about 80 mg/day, about 40 mg/day to about 80 mg/day, about 50 mg/day to about 80 mg/day, about 60 mg/day to about 80 mg/day, about 70 mg/day to about 80 mg/day, about 5 mg/day to about 70 mg/day, about 10 mg/day to about 70 mg/day, about 20 mg/day to about 70 mg/day, about 30 mg/day to about 70 mg/day, about 40 mg/day to about 70 mg/day, about 50 mg/day to about 70 mg/day, or about 60 mg/day to about 70 mg/day of the 4-(3-cyanophenyl)-6-pyridinylpyrimidine derivative (e.g., Compound 1 or Compound 2) or a pharmaceutically acceptable salt thereof.

In some embodiments, the subject is administered a dosage of about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, or about 100 mg/day of the 4-(3-cyanophenyl)-6-pyridinylpyrimidine derivative (e.g., Compound 1 or Compound 2) or a pharmaceutically acceptable salt thereof. In some embodiments, the subject is administered a dosage of about 40 mg/day of the 4-(3-cyanophenyl)-6-pyridinylpyrimidine derivative (e.g., Compound 1 or Compound 2) or a pharmaceutically acceptable salt thereof. In some embodiments, the subject is administered a dosage of about 50 mg/day of the 4-(3-cyanophenyl)-6-pyridinylpyrimidine derivative (e.g., Compound 1 or Compound 2) or a pharmaceutically acceptable salt thereof. In some embodiments, the subject is administered a dosage of about 60 mg/day of the 4-(3-cyanophenyl)-6-pyridinylpyrimidine derivative (e.g., Compound 1 or Compound 2) or a pharmaceutically acceptable salt thereof. Some embodiments are directed to a method of treating a subject having an SUD (e.g., AUD, OUD, and/or a stimulant use disorder such as SUD), wherein the method comprises administering a dosage of about 5 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, or about 100 mg/day of the 4-(3-cyanophenyl)-6-pyridinylpyrimidine derivative (e.g., Compound 1 or Compound 2) or a pharmaceutically acceptable salt thereof.

In some embodiments, the subject is administered the 4-(3-cyanophenyl)-6-pyridinylpyrimidine derivative (e.g., Compound 1 or Compound 2) or a pharmaceutically acceptable salt thereof when the subject is in a fed state (e.g., shortly after a meal.) In some embodiments, the subject is administered the 4-(3-cyanophenyl)-6-pyridinylpyrimidine derivative (e.g., Compound 1 or Compound 2) or a pharmaceutically acceptable salt thereof within about 10 minutes, about 30 minutes, about 1 hour, about 2 hours, about 3 hours, about 4 hours, or about 5 hours of eating a meal.

In some embodiments, the subject is administered the 4-(3-cyanophenyl)-6-pyridinylpyrimidine derivative (e.g., Compound 1 or Compound 2) or a pharmaceutically acceptable salt thereof in progressively increasing dosages so that the subject may acclimate, allowing higher dosages with minimal side effects.

In some embodiments, the subject is administered about ¼ the dosage for 1, 2, 3, 4, or 5 days of administration, then about 2/4 the dosage for 1, 2, 3, 4, or 5 days of administration, then about ¼ the dosage for 1, 2, 3, 4, or 5 days of administration, then the full dosage from then onwards.

In some embodiments, the subject is administered about ⅓ the dosage for 1, 2, 3, 4, or 5 days of administration, then about ⅔ the dosage for 1, 2, 3, 4, or 5 days of administration, then the full dosage from then onwards.

In some embodiments, the subject is administered about ⅓ the dosage for days 1 to 3 of administration, about ⅔ the dosage for days 4 to 6 of administration, and the full dosage from day 7 onwards.

In some embodiments, the subject is administered about ⅓ the dosage over 2 daily administrations for days 1 to 3 of administration, about ⅔ the dosage over 2 daily administrations for days 4 to 6 of administration, the full dosage over 2 daily administrations for days 7 to 13 of administration, and the full dosage in a once daily administration for days 14 onward.

In some embodiments, the subject is administered 50 mg/day over 2 daily administrations for days 1 to 3 of administration, about 100 mg/day over 2 daily administrations for days 4 to 6 of administration, about 150 mg/day over 2 daily administrations for days 7 to 13 of administration, and about 150 mg/day in a once daily administration for days 14 onward.

In some embodiments, the treatment achieves an average plasma concentration of about 10 ng/mL to about 300 ng/mL, about 50 ng/mL to about 300 ng/mL, about 10 ng/ml to about 200 ng/mL, about 10 ng/mL to about 150 ng/mL, about 50 ng/mL to about 150 ng/ml, about 75 ng/mL to about 150 ng/mL, about 100 ng/mL to about 150 ng/ml, about 10 ng/ml to about 100 ng/mL, about 50 ng/mL to about 100 ng/mL, about 75 ng/ml to about 100 ng/ml, about 10 ng/mL to about 75 ng/mL, or about 50 ng/ml to about 75 ng/ml. In some embodiments, the treatment achieves an average plasma concentration of about 100 ng/mL.

### Exemplary Embodiments

Embodiment I-1. A method of treating a subject having alcohol use disorder (AUD) who is at risk of relapse to alcohol use, the method comprising administering to the subject a therapeutically effective amount of a 4-(3-cyanophenyl)-6-pyridinylpyrimidine derivative of the formula

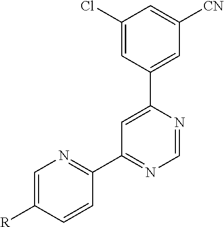

or a pharmaceutically acceptable salt thereof, wherein R is H, Me, F, Cl or cyano;

wherein the administration reduces susceptibility to one or more relapse triggers.

Embodiment I-2. The method of embodiment I-1, wherein the administration results in remission of AUD for at least 3 months.

Embodiment I-3. A method of promoting remission in a subject having alcohol use disorder (AUD), the method comprising administering to the subject a therapeutically effective amount of a 4-(3-cyanophenyl)-6-pyridinylpyrimidine derivative of the formula

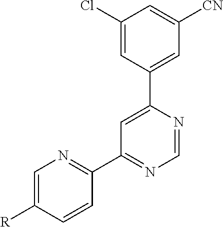

or a pharmaceutically acceptable salt thereof, wherein R is H, Me, F, Cl or cyano;

wherein the administration results in remission for at least 3 months.

Embodiment I-4. The methods of embodiment I-2 and I-3, wherein the administration results in remission for at least 12 months.

Embodiment I-5. The methods embodiments I-1 to I-4, wherein the administration prevents relapse for at least 3 months.

Embodiment I-6. The methods embodiments I-1 to I-5, wherein the administration prevents relapse for at least 12 months.

Embodiment I-7. The methods of embodiment I-5 and I-6, wherein the method further comprises one or more tests to determine relapse.

Embodiment I-8. The method of embodiment I-7, wherein one of the one or more tests to determine relapse is a Timeline-Followback self-reported alcohol consumption assessment.

Embodiment I-9. The methods of embodiment I-7 and I-8, wherein one of the one or more tests to determine relapse is an acute alcohol ingestion test.

Embodiment I-10. The methods of embodiments I-7 to I-9, wherein one of the one or more tests to determine relapse is a chronic alcohol use test.

Embodiment I-11. The methods of embodiments I-1 to I-10, wherein the administration results in the subject having no early-relapse symptoms for at least 3 months.

Embodiment I-12. The methods of embodiments I-1 to I-11, wherein the administration results in the subject having no early-relapse symptoms for at least 12 months.

Embodiment I-13. The methods of embodiments I-1 to I-12, wherein the method further comprises determining if the subject meets the DSM-5 diagnostic criteria of AUD, administering the 4-(3-cyanophenyl)-6-pyridinylpyrimidine derivative (e.g., Compound 1 or Compound 2) or the pharmaceutically acceptable salt thereof if the subject meets the DSM-5 criteria of AUD, and not administering the 4-(3-cyanophenyl)-6-pyridinylpyrimidine derivative (e.g., Compound 1 or Compound 2) or the pharmaceutically acceptable salt thereof if the subject does not meet the DSM-5 diagnostic criteria of AUD.

Embodiment I-14. The methods of embodiments I-1 to I-13, wherein the 4-(3-cyanophenyl)-6-pyridinylpyrimidine derivative (e.g., Compound 1 or Compound 2) or the pharmaceutically acceptable salt thereof is administered to the subject after a period of abstinence by the subject.

Embodiment I-15. The method of embodiment I-14, wherein the method further comprises performing a test to confirm abstinence.

Embodiment I-16. The methods of embodiments I-14 and I-15, wherein the period of abstinence is at least 12 hours and the subject has a breath or blood alcohol level of about 0.

Embodiment I-17. The methods of embodiments I-14 to I-16, wherein the period of abstinence is at least 1 day, at least 3 days, at least 5 days, at least 1 week, at least 2 weeks, at least 3 weeks, at least 1 month, at least 3 months, or at least 6 months.

Embodiment I-18. The methods of embodiments I-1 to I-17, wherein R is F or cyano.

Embodiment I-19. The methods of embodiments I-1 to I-18, wherein the 4-(3-cyanophenyl)-6-pyridinylpyrimidine derivative is

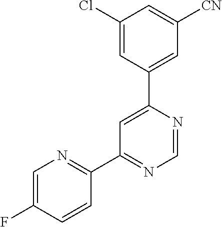 [SMILES: N#Cc1cc(Cl)cc(-c2cc(-c3ccc(F)cn3)ncn2)c1]

or a pharmaceutically acceptable salt thereof.

Embodiment I-20. The methods of embodiments I-1 to I-18, wherein the 4-(3-cyanophenyl)-6-pyridinylpyrimidine derivative is

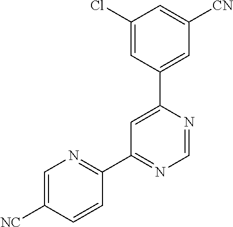 [SMILES: N#Cc1ccc(-c2cc(-c3cc(Cl)cc(C#N)c3)ncn2)nc1]

or a pharmaceutically acceptable salt thereof.

Embodiment I-21. The methods of embodiments I-1 to I-20, wherein the treatment achieves an average plasma concentration of about 100 ng/mL.

Embodiment I-22. The methods of embodiments I-1 to I-21, wherein the subject is administered a dosage of about 30 mg/day to about 300 mg/day, about 50 mg/day to about 200 mg/day, about 50 mg/day to about 120 mg/day, about 50 mg/day to about 100 mg/day, about 60 mg/day to about 90 mg/day, about 60 mg/day to about 80 mg/day, or about 60 mg/day to about 70 mg/day of the 4-(3-cyanophenyl)-6-pyridinylpyrimidine derivative (e.g., Compound 1 or Compound 2) or the pharmaceutically acceptable salt thereof.

Embodiment I-23. The methods of embodiments I-1 to I-21, wherein the subject is administered a dosage of about 50 mg/day, about 60 mg/day, about 70 mg/day, about 80 mg/day, about 90 mg/day, about 100 mg/day, about 110 mg/day, about 120 mg/day, about 200 mg/day, or about 300 mg/day of the 4-(3-cyanophenyl)-6-pyridinylpyrimidine derivative (e.g., Compound 1 or Compound 2) or the pharmaceutically acceptable salt thereof.

Embodiment I-24. The methods of embodiments I-22 and I-23, wherein the subject is administered about ⅓ the dosage for days 1 to 3 of administration, about ⅔ the dosage for days 4 to 6 of administration, and the full dosage from day 7 onwards.

Embodiment I-25. The method of embodiment I-24, wherein the subject is administered about ⅓ the dosage over 2 daily administrations for days 1 to 3 of administration, about ⅔ the dosage over 2 daily administrations for days 4 to 6 of administration, the full dosage over 2 daily administrations for days 7 to 13 of administration, and the full dosage in a once daily administration for days 14 onward.

Embodiment I-26. The method of embodiment I-25, wherein the subject is administered 50 mg/day over 2 daily administrations for days 1 to 3 of administration, about 100 mg/day over 2 daily administrations for days 4 to 6 of administration, about 150 mg/day over 2 daily administrations for days 7 to 13 of administration, and about 150 mg/day in a once daily administration for days 14 onward.

Embodiment I-27. Use of a 4-(3-cyanophenyl)-6-pyridinylpyrimidine derivative in a treatment for reducing susceptibility to one or more relapse triggers, wherein the treatment comprises administering a therapeutically effective amount of the 4-(3-cyanophenyl)-6-pyridinylpyrimidine derivative to a subject having alcohol use disorder (AUD) who is at risk of relapse to alcohol use;

wherein the 4-(3-cyanophenyl)-6-pyridinylpyrimidine derivative is a compound of the formula

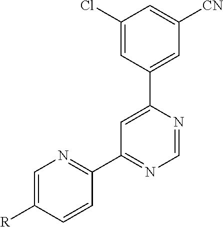

or a pharmaceutically acceptable salt thereof, wherein R is H, Me, F, Cl or cyano.

Embodiment I-28. Use of a 4-(3-cyanophenyl)-6-pyridinylpyrimidine derivative in the manufacture of a medicament for treatment for reducing susceptibility to one or more relapse triggers, wherein the treatment comprises administering the medicament to a subject having alcohol use disorder (AUD) who is at risk of relapse to alcohol use;

wherein the 4-(3-cyanophenyl)-6-pyridinylpyrimidine derivative is a compound of the formula

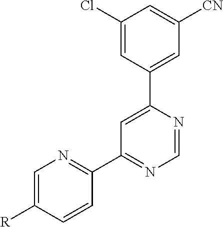

or a pharmaceutically acceptable salt thereof, wherein R is H, Me, F, Cl or cyano.

Embodiment I-29. A 4-(3-cyanophenyl)-6-pyridinylpyrimidine derivative for use in a method of reducing susceptibility to one or more relapse triggers, the method comprising administering a therapeutically effective amount of the 4-(3-cyanophenyl)-6-pyridinylpyrimidine derivative to a subject having alcohol use disorder (AUD) who is at risk of relapse to alcohol use;

wherein the 4-(3-cyanophenyl)-6-pyridinylpyrimidine derivative is a compound of the formula

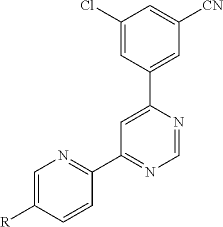

or a pharmaceutically acceptable salt thereof, wherein R is H, Me, F, Cl or cyano.

Embodiment I-30. A medicament for treatment for reducing susceptibility to one or more relapse triggers, the medicament comprising a 4-(3-cyanophenyl)-6-pyridinylpyrimidine derivative, wherein the treatment comprises administering the medicament to deliver a therapeutically effective amount of the 4-(3-cyanophenyl)-6-pyridinylpyrimidine derivative to a subject having alcohol use disorder (AUD) who is at risk of relapse to alcohol use;

wherein the 4-(3-cyanophenyl)-6-pyridinylpyrimidine derivative is a compound of the formula

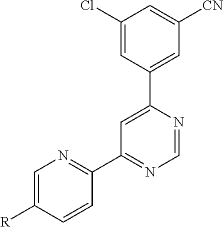

or a pharmaceutically acceptable salt thereof, wherein R is H, Me, F, Cl or cyano.

Embodiment I-31. The uses, the 4-(3-cyanophenyl)-6-pyridinylpyrimidine derivatives, and the medicaments of embodiments I-27 to I-30, wherein the administration results in remission of AUD for at least 3 months.

Embodiment I-32. Use of a 4-(3-cyanophenyl)-6-pyridinylpyrimidine derivative in the manufacture of a medicament for treatment for promoting remission of alcohol use disorder (AUD), wherein the treatment comprises administering the medicament to a subject having AUD;

wherein the 4-(3-cyanophenyl)-6-pyridinylpyrimidine derivative is a compound of the formula

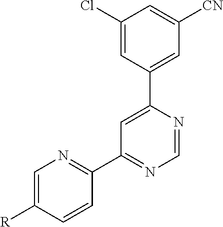

or a pharmaceutically acceptable salt thereof, wherein R is H, Me, F, Cl or cyano.

Embodiment I-33. Use of a 4-(3-cyanophenyl)-6-pyridinylpyrimidine derivative in a treatment for promoting remission of alcohol use disorder (AUD), wherein the treatment comprises administering a therapeutically effective amount of the 4-(3-cyanophenyl)-6-pyridinylpyrimidine derivative to a subject having AUD;

wherein the 4-(3-cyanophenyl)-6-pyridinylpyrimidine derivative is a compound of the formula

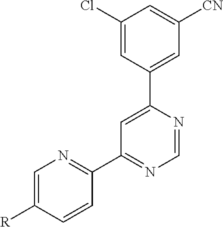

or a pharmaceutically acceptable salt thereof, wherein R is H, Me, F, Cl or cyano.

Embodiment I-34. A 4-(3-cyanophenyl)-6-pyridinylpyrimidine derivative for use in a method for promoting remission of alcohol use disorder (AUD), the method comprising administering a therapeutically effective amount of the 4-(3-cyanophenyl)-6-pyridinylpyrimidine derivative to a subject having AUD;

wherein the 4-(3-cyanophenyl)-6-pyridinylpyrimidine derivative is a compound of the formula

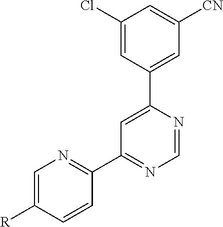

or a pharmaceutically acceptable salt thereof, wherein R is H, Me, F, Cl or cyano.

Embodiment I-35. A medicament for treatment for promoting remission of alcohol use disorder (AUD), the medicament comprising a 4-(3-cyanophenyl)-6-pyridinylpyrimidine derivative, wherein the treatment comprises administering the medicament to deliver a therapeutically effective amount of the 4-(3-cyanophenyl)-6-pyridinylpyrimidine derivative to a subject having AUD;

wherein the 4-(3-cyanophenyl)-6-pyridinylpyrimidine derivative is a compound of the formula

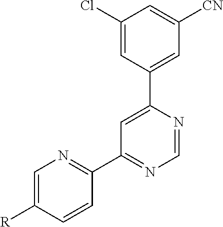

or a pharmaceutically acceptable salt thereof, wherein R is H, Me, F, Cl or cyano.

Embodiment I-36. The uses, the 4-(3-cyanophenyl)-6-pyridinylpyrimidine derivatives, and the medicaments of embodiments I-31 to I-35, wherein the administration results in remission for at least 12 months.

Embodiment I-37. The uses, the 4-(3-cyanophenyl)-6-pyridinylpyrimidine derivatives, and the medicaments of embodiments I-27 to I-36, wherein the administration prevents relapse for at least 3 months.

Embodiment I-38. The uses, the 4-(3-cyanophenyl)-6-pyridinylpyrimidine derivatives, and the medicaments of embodiments I-27 to I-37, wherein the administration prevents relapse for at least 12 months.

Embodiment I-39. The uses, the 4-(3-cyanophenyl)-6-pyridinylpyrimidine derivatives, and the medicaments of embodiments I-37 and I-38, wherein the method further comprises one or more tests to determine relapse.

Embodiment I-40. The use, the 4-(3-cyanophenyl)-6-pyridinylpyrimidine derivative, and the medicament of embodiment I-39, wherein one of the one or more tests to determine relapse is a Timeline-Followback self-reported alcohol consumption assessment.

Embodiment I-41. The uses, the 4-(3-cyanophenyl)-6-pyridinylpyrimidine derivatives, and the medicaments of embodiments I-39 and I-40, wherein one of the one or more tests to determine relapse is an acute alcohol ingestion test.

Embodiment I-42. The uses, the 4-(3-cyanophenyl)-6-pyridinylpyrimidine derivatives, and the medicaments of embodiments I-39 to I-41, wherein one of the one or more tests to determine relapse is a chronic alcohol use test.

Embodiment I-43. The uses, the 4-(3-cyanophenyl)-6-pyridinylpyrimidine derivatives, and the medicaments of embodiments I-27 to I-42, wherein the administration results in the subject having no early-relapse symptoms for at least 3 months.

Embodiment I-44. The uses, the 4-(3-cyanophenyl)-6-pyridinylpyrimidine derivatives, and the medicaments of embodiments I-27 to I-43, wherein the administration results in the subject having no early-relapse symptoms for at least 12 months.

Embodiment I-45. The uses, the 4-(3-cyanophenyl)-6-pyridinylpyrimidine derivatives, and the medicaments of embodiments I-27 to I-44, wherein the method further comprises determining if the subject meets the DSM-5 diagnostic criteria of AUD, administering the 4-(3-cyanophenyl)-6-pyridinylpyrimidine derivative (e.g., Compound 1 or Compound 2) or a pharmaceutically acceptable salt thereof if the subject meets the DSM-5 criteria of AUD, and not administering the 4-(3-cyanophenyl)-6-pyridinylpyrimidine derivative (e.g., Compound 1 or Compound 2) or a pharmaceutically acceptable salt thereof if the subject does not meet the DSM-5 diagnostic criteria of AUD.

Embodiment I-46. The uses, the 4-(3-cyanophenyl)-6-pyridinylpyrimidine derivatives, and the medicaments of embodiments I-27 to I-45, wherein the 4-(3-cyanophenyl)-6-pyridinylpyrimidine derivative (e.g., Compound 1 or Compound 2) or the pharmaceutically acceptable salt thereof is administered to the subject after a period of abstinence by the subject.

Embodiment I-47. The use, the 4-(3-cyanophenyl)-6-pyridinylpyrimidine derivative, and the medicament of embodiment I-46, wherein the method further comprises performing a test to confirm abstinence.

Embodiment I-48. The uses, the 4-(3-cyanophenyl)-6-pyridinylpyrimidine derivatives, and the medicaments of embodiments I-46 and I-47, wherein the period of abstinence is at least 12 hours and the subject has a breath or blood alcohol level of about 0.

Embodiment I-49. The uses, the 4-(3-cyanophenyl)-6-pyridinylpyrimidine derivatives, and the medicaments of embodiments I-46 to I-48, wherein the period of abstinence is at least 1 day, at least 3 days, at least 5 days, at least 1 week, at least 2 weeks, at least 3 weeks, at least 1 month, at least 3 months, or at least 6 months.

Embodiment I-50. The uses, the 4-(3-cyanophenyl)-6-pyridinylpyrimidine derivatives, and the medicaments of embodiments I-27 to I-49, wherein R is F or cyano.

Embodiment I-51. The uses, the 4-(3-cyanophenyl)-6-pyridinylpyrimidine derivatives, and the medicaments of embodiments I-27 to I-50, wherein the 4-(3-cyanophenyl)-6-pyridinylpyrimidine derivative is

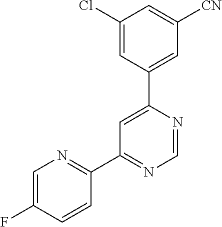 [SMILES: N#Cc1cc(Cl)cc(-c2cc(-c3ccc(F)cn3)ncn2)c1]

or a pharmaceutically acceptable salt thereof.

Embodiment I-52. The uses, the 4-(3-cyanophenyl)-6-pyridinylpyrimidine derivatives, and the medicaments of embodiments I-27 to I-50, wherein the 4-(3-cyanophenyl)-6-pyridinylpyrimidine derivative is

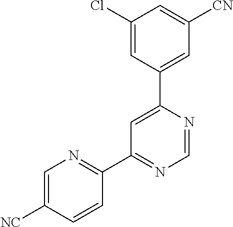 [SMILES: N#Cc1ccc(-c2cc(-c3cc(Cl)cc(C#N)c3)ncn2)nc1]

or a pharmaceutically acceptable salt thereof.

Embodiment I-53. The uses, the 4-(3-cyanophenyl)-6-pyridinylpyrimidine derivatives, and the medicaments of embodiments I-27 to I-52, wherein the treatment achieves an average plasma concentration of about 100 ng/mL.

Embodiment I-54. The uses, the 4-(3-cyanophenyl)-6-pyridinylpyrimidine derivatives, and the medicaments of embodiments I-27 to I-53, wherein the subject is administered a dosage of about 30 mg/day to about 300 mg/day, about 50 mg/day to about 200 mg/day, about 50 mg/day to about 120 mg/day, about 50 mg/day to about 100 mg/day, about 60 mg/day to about 90 mg/day, about 60 mg/day to about 80 mg/day, or about 60 mg/day to about 70 mg/day of the 4-(3-cyanophenyl)-6-pyridinylpyrimidine derivative (e.g., Compound 1 or Compound 2) or a pharmaceutically acceptable salt thereof.

Embodiment I-55. The uses, the 4-(3-cyanophenyl)-6-pyridinylpyrimidine derivatives, and the medicaments of embodiments I-27 to I-53, wherein the subject is administered a dosage of about 50 mg/day, about 60 mg/day, about 70 mg/day, about 80 mg/day, about 90 mg/day, about 100 mg/day, about 110 mg/day, about 120 mg/day, about 200 mg/day, or about 300 mg/day of the 4-(3-cyanophenyl)-6-pyridinylpyrimidine derivative (e.g., Compound 1 or Compound 2) or a pharmaceutically acceptable salt thereof.

Embodiment I-56. The uses, the 4-(3-cyanophenyl)-6-pyridinylpyrimidine derivatives, and the medicaments of embodiments I-54 and I-55, wherein the subject is administered about ⅓ the dosage for days 1 to 3 of administration, about ⅔ the dosage for days 4 to 6 of administration, and the full dosage from day 7 onwards.

Embodiment I-57. The use, the 4-(3-cyanophenyl)-6-pyridinylpyrimidine derivative, and the medicament of embodiment I-56, wherein the subject is administered about ⅓ the dosage over 2 daily administrations for days 1 to 3 of administration, about ⅔ the dosage over 2 daily administrations for days 4 to 6 of administration, the full dosage over 2 daily administrations for days 7 to 13 of administration, and the full dosage in a once daily administration for days 14 onward.

Embodiment I-58. The use, the 4-(3-cyanophenyl)-6-pyridinylpyrimidine derivative, and the medicament of embodiment I-57, wherein the subject is administered 50 mg/day over 2 daily administrations for days 1 to 3 of administration, about 100 mg/day over 2 daily administrations for days 4 to 6 of administration, about 150 mg/day over 2 daily administrations for days 7 to 13 of administration, and about 150 mg/day in a once daily administration for days 14 onward.

### Set II

Embodiment II-1. A method of treating a subject having a substance use disorder (SUD) who is at risk of relapse to substance use, the method comprising administering to the subject a therapeutically effective amount of a compound of the formula

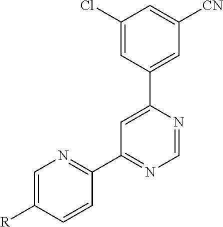

or a pharmaceutically acceptable salt thereof, wherein R is H, Me, F, Cl or cyano;

wherein the administration reduces susceptibility to one or more relapse triggers.

Embodiment II-2. Use of a compound in a treatment for reducing susceptibility to one or more relapse triggers, wherein the treatment comprises administering a therapeutically effective amount of the compound to a subject having a substance use disorder (SUD) who is at risk of relapse to substance use;

wherein the compound is of the formula

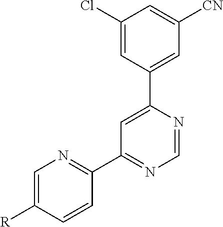

or a pharmaceutically acceptable salt thereof, wherein R is H, Me, F, Cl or cyano.

Embodiment II-3. Use of a compound in the manufacture of a medicament for treatment for reducing susceptibility to one or more relapse triggers, wherein the treatment comprises administering the medicament to a subject having a substance use disorder (SUD) who is at risk of relapse to substance use;

wherein the compound is of the formula

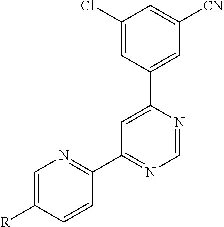

or a pharmaceutically acceptable salt thereof, wherein R is H, Me, F, Cl or cyano.

Embodiment II-4. A compound for use in a method of reducing susceptibility to one or more relapse triggers, the method comprising administering a therapeutically effective amount of the compound to a subject having a substance use disorder (SUD) who is at risk of relapse to substance use;

wherein the compound is of the formula

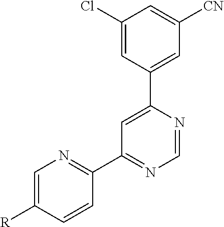

or a pharmaceutically acceptable salt thereof, wherein R is H, Me, F, Cl or cyano.

Embodiment II-5. A medicament for treatment for reducing susceptibility to one or more relapse triggers, the medicament comprising a compound, wherein the treatment comprises administering the medicament to deliver a therapeutically effective amount of the compound to a subject having a substance use disorder (SUD) who is at risk of relapse to substance use;

wherein the compound is of the formula

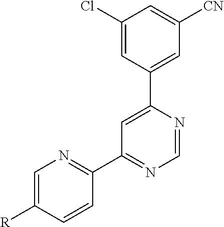

or a pharmaceutically acceptable salt thereof, wherein R is H, Me, F, Cl or cyano.

Embodiment II-6. The method, the use, the compound, or the medicament of any one of embodiments II-1 to II-5, wherein the administration results in remission of the SUD for at least 3 months.

Embodiment II-7. A method of promoting remission in a subject having a substance use disorder (SUD), the method comprising administering to the subject a therapeutically effective amount of a compound of the formula

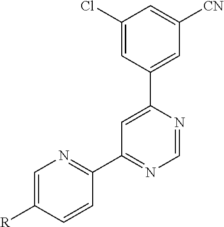

or a pharmaceutically acceptable salt thereof, wherein R is H, Me, F, Cl or cyano;

wherein the administration results in remission for at least 3 months.

Embodiment II-8. Use of a compound in the manufacture of a medicament for treatment for promoting remission of a substance use disorder (SUD), wherein the treatment comprises administering the medicament to a subject having the SUD;

wherein the compound is of the formula

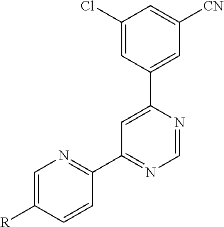

or a pharmaceutically acceptable salt thereof, wherein R is H, Me, F, Cl or cyano.

Embodiment II-9. Use of a compound in a treatment for promoting remission of a substance use disorder (SUD), wherein the treatment comprises administering a therapeutically effective amount of the compound to a subject having the SUD;

wherein the compound is of the formula

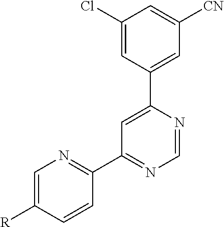

or a pharmaceutically acceptable salt thereof, wherein R is H, Me, F, Cl or cyano.

Embodiment II-10. A compound for use in a method for promoting remission of a substance use disorder (SUD), the method comprising administering a therapeutically effective amount of the compound to a subject having the SUD;

wherein the compound is of the formula

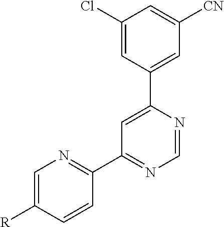

or a pharmaceutically acceptable salt thereof, wherein R is H, Me, F, Cl or cyano.

Embodiment II-11. A medicament for treatment for promoting remission of substance use disorder (SUD), the medicament comprising a compound, wherein the treatment comprises administering the medicament to deliver a therapeutically effective amount of the compound to a subject having the SUD;

wherein the compound is of the formula

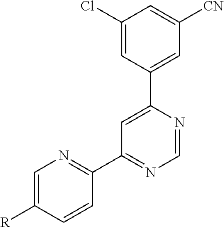

or a pharmaceutically acceptable salt thereof, wherein R is H, Me, F, Cl or cyano.

Embodiment II-12. The method, the use, the compound for use, and the medicament of embodiments II-7 to II-11, wherein the administration results in remission for at least 12 months.

Embodiment II-13. The method, the use, the compound for use, and the medicament of embodiments II-1 to II-12, wherein the administration prevents relapse for at least 3 months.

Embodiment II-14. The method, the use, the compound for use, and the medicament of embodiments II-1 to II-13, wherein the administration prevents relapse for at least 12 months.

Embodiment II-15. The method, the use, the compound for use, and the medicament of embodiment II-13 and II-14, wherein the method or the treatment further comprises one or more tests to determine relapse.

Embodiment II-16. The method, the use, the compound for use, and the medicament of embodiment II-15, wherein one of the one or more tests to determine relapse is a Timeline-Followback self-reported substance consumption assessment.

Embodiment II-17. The method, the use, the compound for use, and the medicament of embodiment II-15, wherein one of the one or more tests to determine relapse is a urine drug screening test, a saliva drug test, or a hair follicle drug test.

Embodiment II-18. The method, the use, the compound for use, and the medicament of embodiment II-15, wherein one of the one or more tests to determine relapse is an acute alcohol ingestion test.

Embodiment II-19. The method, the use, the compound for use, and the medicament of embodiments II-15, wherein one of the one or more tests to determine relapse is a chronic alcohol use test.

Embodiment II-20. The method, the use, the compound for use, and the medicament of embodiments II-1 to II-19, wherein the administration results in the subject having no early-relapse symptoms for at least 3 months.

Embodiment II-21. The method, the use, the compound for use, and the medicament of embodiments II-1 to II-20, wherein the administration results in the subject having no early-relapse symptoms for at least 12 months.

Embodiment II-22. The method, the use, the compound for use, and the medicament of embodiments II-1 to II-21, wherein the method or the treatment further comprises determining if the subject meets the DSM-5 diagnostic criteria of the SUD, administering the compound or the pharmaceutically acceptable salt thereof if the subject meets the DSM-5 criteria of the SUD, and not administering the compound or the pharmaceutically acceptable salt thereof if the subject does not meet the DSM-5 diagnostic criteria of the SUD.

Embodiment II-23. The method, the use, the compound for use, and the medicament of embodiments II-1 to II-22, wherein the compound or the pharmaceutically acceptable salt thereof is administered to the subject after a period of abstinence by the subject.

Embodiment II-24. The method, the use, the compound for use, and the medicament of embodiment II-23, wherein the method or the treatment further comprises performing a test to confirm abstinence.

Embodiment II-25. The method, the use, the compound for use, and the medicament of embodiment II-23 and II-24, wherein the period of abstinence is at least 12 hours and the subject has a breath or blood alcohol level of about 0.

Embodiment II-26. The method, the use, the compound for use, and the medicament of embodiments II-23 to II-25, wherein the period of abstinence is at least 1 day, at least 3 days, at least 5 days, at least 1 week, at least 2 weeks, at least 3 weeks, at least 1 month, at least 3 months, or at least 6 months.

Embodiment II-27. The method, the use, the compound for use, and the medicament of embodiments II-1 to II-26, wherein R is F or cyano.

Embodiment II-28. The method, the use, the compound for use, and the medicament of embodiments II-1 to II-27, wherein the compound is

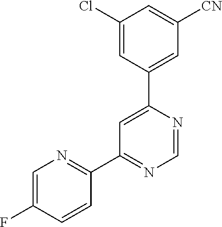 [SMILES: N#Cc1cc(Cl)cc(-c2cc(-c3ccc(F)cn3)ncn2)c1]

or a pharmaceutically acceptable salt thereof.

Embodiment II-29. The method, the use, the compound for use, and the medicament of embodiments II-1 to II-27, wherein the compound is

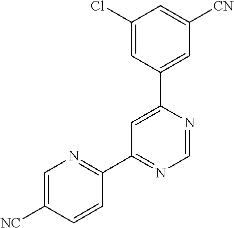 [SMILES: N#Cc1ccc(-c2cc(-c3cc(Cl)cc(C#N)c3)ncn2)nc1]

or a pharmaceutically acceptable salt thereof.

Embodiment II-30. The method, the use, the compound for use, and the medicament of embodiments II-1 to II-29, wherein the method or the treatment achieves an average plasma concentration of about 10 ng/ml to about 300 ng/mL.

Embodiment II-31. The method, the use, the compound for use, and the medicament of embodiments II-1 to II-30, wherein the subject is administered a dosage of about 5 mg/day to about 300 mg/day, about 30 mg/day to about 300 mg/day, about 50 mg/day to about 300 mg/day, about 5 mg/day to about 200 mg/day, about 30 mg/day to about 200 mg/day, about 50 mg/day to about 200 mg/day, about 30 mg/day to about 120 mg/day, about 50 mg/day to about 120 mg/day, about 30 mg/day to about 100 mg/day, about 50 mg/day to about 100 mg/day, about 30 mg/day to about 90 mg/day, about 50 mg/day to about 90 mg/day, about 30 mg/day to about 80 mg/day, about 50 mg/day to about 80 mg/day, about 30 mg/day to about 70 mg/day, about 50 mg/day to about 70 mg/day, about 60 mg/day to about 90 mg/day, about 60 mg/day to about 80 mg/day, or about 60 mg/day to about 70 mg/day of the compound or the pharmaceutically acceptable salt thereof.

Embodiment II-32. The method, the use, the compound for use, and the medicament of embodiments II-1 to II-31, wherein the subject is administered a dosage of about 10 mg/day, about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 200 mg/day, or about 300 mg/day of the compound or the pharmaceutically acceptable salt thereof.

Embodiment II-33. The method, the use, the compound for use, and the medicament of embodiments II-31 to II-32, wherein the subject is administered about ⅓ the dosage for days 1 to 3 of administration, about ⅔ the dosage for days 4 to 6 of administration, and the full dosage from day 7 onwards.

Embodiment II-34. The method, the use, the compound for use, and the medicament of embodiment II-33, wherein the subject is administered about ⅓ the dosage over 2 daily administrations for days 1 to 3 of administration, about ⅔ the dosage over 2 daily administrations for days 4 to 6 of administration, the full dosage over 2 daily administrations for days 7 to 13 of administration, and the full dosage in a once daily administration for days 14 onward.

Embodiment II-35. The method, the use, the compound for use, and the medicament of embodiment II-34, wherein the subject is administered 50 mg/day over 2 daily administrations for days 1 to 3 of administration, about 100 mg/day over 2 daily administrations for days 4 to 6 of administration, about 150 mg/day over 2 daily administrations for days 7 to 13 of administration, and about 150 mg/day in a once daily administration for days 14 onward.

Embodiment II-36. The method, the use, the compound for use, and the medicament of embodiments II-1 to II-35, wherein the SUD is alcohol use disorder (AUD).

Embodiment II-37. The method, the use, the compound for use, and the medicament of embodiments II-1 to II-17, II-20 to II-24, and II-26 to II-35, wherein the SUD is opioid use disorder (OUD).

Embodiment II-38. The method, the use, the compound for use, and the medicament of embodiments II-1 to II-17, II-20 to II-24, and II-26 to II-35, wherein the SUD is a stimulant use disorder.

Embodiment II-39. The method, the use, the compound for use, and the medicament of embodiment II-38, wherein the stimulant use disorder is cocaine use disorder (CUD).

### EXAMPLES

The following examples are provided to illustrate the present disclosure and should not be construed as limiting thereof. In these examples, all parts and percentages are by weight, unless otherwise noted.

### Example 1—Effects of Compound 1 on Home-Cage Alcohol Drinking, Positive Reinforcement Function of Alcohol, and Cue-Induced Reinstatement of Alcohol Seeking (Relapse)

The following is an additional example concerning effects of Compound 1 on home-cage alcohol drinking, on the positive reinforcement functions of alcohol, and relapse.

### SUMMARY

Background: The purpose of this Research Objective was to investigate the preclinical efficacy of Compound 1, a novel mGluR5 negative allosteric modulator (NAM), to reduce: 1) chronic alcohol drinking; 2) positive reinforcement function of alcohol as modeled by operant self-administration; and 3) cue-induced reinstatement of operant alcohol-seeking behavior as a model of relapse in Wistar rats. Compound 1 is brain penetrant and shows high affinity for mGluR5 following systemic administration in rats, mice, and primates.

Methods and Results: Acute systemic administration of Compound 1 (0-10 mg/kg, IP) produced a dose-dependent reduction in home-cage sweetened alcohol intake. Comparison of Compound 1 (3 mg/kg) to the mGluR5 NAM MPEP (3 mg/kg) and the opiate antagonist naltrexone (3 mg/kg) showed that all compounds significantly reduced home-cage alcohol intake after 1 hr of access as compared to vehicle control with no statistically significant difference between the 3 test compounds. After 24-hrs of alcohol access, only Compound 1 showed efficacy for reducing sweetened alcohol intake. Overall, there were trends toward reductions in parallel water intake but no dose of Compound 1 reached statistical significance on this control measure. Compound 1 had no effect on habituation to a novel environment, total motor activity, or anxiety-like behavior when evaluated in an open-field test suggesting that effects on home-cage alcohol drinking and operant self-administration were not associated with non-specific behavioral effects. Repeated treatment with Compound 1 (3 mg/kg) for 7 days also reduced average alcohol intake in the home-cage over the 1-week period, but only during the first hour of access. Importantly, Compound 1 also reduced operant alcohol self-administration (e.g., number of alcohol reinforced lever presses) in a dose-dependent manner, which indicates blockade of the positive reinforcing effects of alcohol. Finally, Compound 1 (10 mg/kg) blocked cue-induced reinstatement of alcohol-seeking behavior, which is an animal model of relapse that is triggered by exposure to environmental stimuli previously paired with alcohol use.

Conclusions: Compound 1 showed preclinical efficacy for reducing voluntary alcohol drinking, operant alcohol self-administration (reinforcement/reward), and cue-induced reinstatement (relapse) in Wistar rats. When administered acutely, Compound 1 significantly decreased alcohol drinking and operant self-administration, and was equally efficacious when compared to naltrexone, which is FDA approved for treating AUD. Compound 1 showed improved efficacy to reduce palatable alcohol intake after a 24-hr access period. Control experiments (water intake, motor activity, anxiety-like performance) found no nonspecific effects that might account for reductions in alcohol drinking, self-administration, or relapse-like behavior. These preclinical results support the conclusion that Compound 1 may be useful in the medical management of chronic alcohol drinking and relapse.

Specific Goals of these Studies

The purpose of Research Objective 3.1 was to evaluate the preclinical efficacy of the novel mGluR5 negative allosteric modulator Compound 1 (3-chloro-5-[6-(5-fluoropyridin-2-yl)pyrimidin-4-yl]benzonitrile; AKA HTL0014242) for reducing chronic voluntary alcohol drinking in rats. Two studies were conducted under two separate Research Objectives-Objective 3.1 and Objective 3.2.

Research Objective 3.1. RO 3.1 had two goals. First, Objective 3.1a was to evaluate preclinical efficacy of acute treatment with Compound 1 (0-10 mg/kg) to reduce voluntary home-cage alcohol drinking was evaluated in Study 3.1.a. The behavioral effects of Compound 1 were compared to the mGluR5 negative modulator MPEP and the opiate antagonist naltrexone, which is FDA approved for treatment of AUD. In addition, an effective dose of Compound 1 was evaluated for potential nonspecific behavioral effects in open-field locomotor chambers that evaluate motor function and other naturalistic rodent behaviors. Second, Objective 3.1b was to test an effective dose of Compound 1 (3 mg/kg) identified in Study 3.1.a to evaluate effects of a repeated treatment protocol.

Research Objective 3.2. Under Research Objective 3.2, studies were conducted to evaluate the preclinical efficacy of Compound 1 to reduce the positive reinforcing and relapse inducing effects of alcohol. First, preclinical efficacy data were collected on the ability of pretreatment with an acute dose range of Compound 1 to reduce the positive reinforcing effects of alcohol during chronic operant self-administration. We also sought to assess efficacy of repeated treatment with a single effective dose of Compound 1 determined from the acute dose-response curve. Second, the final objective of this project was to assess the preclinical efficacy of Compound 1 to inhibit relapse-like behavior. The cue-induced reinstatement method was used to model relapse that occurs when abstinent individuals encounter environmental stimuli that were previously paired with drug use. These “cues” are widely known to induce craving and relapse in abstinent drug users, including AUD patients. We have shown that the mGluR5 NAM MPEP blocks cue-induced reinstatement in rats and predict that Compound 1 will show similar efficacy.

### Materials and Methods

Animals: Male Wistar rats (Charles River Laboratories, INC) were used in these studies. Rat housing and testing was conducted at the University of North Carolina at Chapel Hill (UNC-CH) School of Medicine in a state-of-the-art rodent vivarium approved by the Association for Assessment and Accreditation of Laboratory Animal Care International (AAALAC). Rat health was monitored daily by study investigators and veterinarians in the Division of Comparative Medicine UNC. All experimental procedures were carried out in accordance with the NIH Guide to Care and Use of Laboratory Animals (Committee for the Update of the Guide for the Care and Use of Laboratory Animals, 2011) and institutional guidelines.

Rats were housed individually in standard Plexiglas cages with standard rodent food and water available ad libitum. Upon arrival from the vendor, rats were given 7 days to acclimate to the vivarium during which they were weighed and handled by an investigator each day to monitor general health and habituate them to human contact. Rats were then weighed each day within 1-h of experimental procedures to calculate drug dosages relative to body weight. Rats showed typical growth and development with body weight throughout the experimental procedures. The colony was maintained at 21÷1° C. on a 12-hour light/dark cycle, with the lights on from 10 AM-10 PM. All behavioral experiments were conducted during the light portion of the light cycle.

Research Objectives 3.1a and 3.1b—Effects of Acute Compound 1 on Home-Cage Alcohol Drinking (Acute and Repeated Treatment)

Home-cage alcohol drinking method. Oral alcohol and water intake were examined using a two-bottle choice protocol, which is standard in the field and commonly used in our laboratory. Male Wistar rats (n=12) were first adapted to drinking from a 50 ml conical tube fitted with a standard stainless-steel ball bearing sipper tube that limits spillage. Then, two bottles were place on the cage daily. One bottle contained an alcohol solution and the other contained water. Bottles containing fresh solutions were weighed daily before and after a 24 hr access period. After drug treatment, bottles were weighed after 1 hr and 24 hrs of access. Each week, rats had 4-5 days of sweetened alcohol and water available and the remaining days only water.

Since Wistar rats do not voluntarily consume pharmacologically relevant doses of alcohol, they were trained to drink alcohol using the well-characterized and widely-used sucrose fading technique. Briefly, rats were first exposed to alcohol (10% v/v)+sucrose (5% w/v). versus water in the two bottles. Then, over a period of 6-weeks, the concentrations of alcohol and sucrose were gradually adjusted to alcohol (8% v/v)+sucrose (2% w/v) to achieve intake of pharmacologically relevant doses of alcohol. Rats were maintained at this concentration for the duration of the experiment for both acute (3.1.a) and chronic (3.1.b) home-cage drinking studies. Use of sweetened alcohol solutions in preclinical research provides face validity to the binge-intoxication stage of alcohol addiction in humans where sweeteners are commonly added to alcohol.

Compound 1 treatment. After establishing baseline intake of sweetened alcohol and water, rats were habituated to i.p. injections. All rats (N=12) were administered 3 habituation injections of vehicle (10% w/v kolliphor in BCD (10%)) prior to the Compound 1 dose response curve and alcohol intake was measured. Then, the Compound 1 dose response curve was conducted with dose order randomized according to a modified Latin-Square design. Rats were given a minimum of 1-2 days between injections to ensure normal drinking was re-established in absence of drug administration. Each dose of Compound 1 (0-10 mg/kg) was administered via i.p. injection and rats were placed back in the home-cage for 1 hr prior to the return of sweetened alcohol and water. Alcohol intake was measured after 1-h and 24-hrs of access in grams by weighing the drinking bottles. Data were excluded from 1 rat that emptied both bottles fully each day without drinking the solutions; thus, results are presented for a final group of N=11 rats.

Locomotor activity: testing for nonspecific behavioral effects. After completion of the Compound 1 dose response curve, potential nonspecific effects of Compound 1 (3 mg/kg) were assessed using the open-field locomotor test. This test evaluates potential effects on motor activity and anxiety-like behavior. Rats were administered Compound 1 (0 or 3 mg/kg, i.p.) in a randomized counterbalanced order. After a 1-hr pretreatment period (matching the pretreatment period used in the drinking study), rats were placed in the open-field apparatus and locomotor activity was computer-monitored for 1-hr. To assess anxiety-like behavior, activity in the center zone of the open field was analyzed.

Efficacy comparisons to MPEP and Naltrexone. Following locomotor testing, effects of the mGluR5 negative modulator MPEP (3 mg/kg, i.p.) and the opiate antagonist naltrexone (3 mg/kg, i.p.) were evaluated on alcohol drinking as positive controls. Drugs were administered on separate days in a counter-balanced order.

Repeated treatment with Compound 1. To evaluate effects of repeated treatment with Compound 1 on home-cage alcohol drinking, baseline intake of sweetened alcohol and water, and habituation to i.p. injections was conducted as described above in a separate group of male Wistar rats (n=12). During habituation to i.p. injections, rats were moved to a 7 day per week alcohol access schedule to allow for chronic daily treatment. Rats were then administered vehicle (4 consecutive days), Compound 1 (3 mg/kg, 7 consecutive days), then vehicle (4 consecutive days) according to an A-B-A experimental design for a total of 15 days. Access to alcohol was provided 1-hr after vehicle or Compound 1 injection. Alcohol and water intake were measured as described above after 1-hr and 24-hrs of access. Data were excluded from one rat that failed to consume alcohol; thus, results are reported for a final group of N=11 rats.

Research Objective 3.2a and 3.2b—Effect of Compound 1 on Alcohol Reinforcement (Reward) and Relapse

### Behavioral Apparatus

Twelve operant conditioning chambers designed for rats (30.5 cm×24.1 cm×21.0 cm; Med Associates, Georgia, VT) were located within sound-attenuating cubicles in a devoted room to limit extraneous potential impact by extraneous environmental variables. Each cubicle was equipped with an exhaust fan that provided ventilation and masked external sounds. The chambers were electronically interfaced to a Microsoft Windows based computer that was programmed using investigator developed software to control sessions and record number of lever press responses at 100-ms intervals.

The right wall of each chamber contained a liquid receptacle and a response lever (Med Associates) that was termed the active lever. Lever press responses on the active lever activated a syringe pump (PHM-100, Med Associates) that delivered 0.1 ml of alcohol solution (ethanol 8% v/v+sucrose 2% w/v) into the receptacle over a 1.66-s period. Head entries into the liquid receptacle were recorded when an infrared photo beam was broken. A stimulus light located above the response lever was illuminated during pump activation. Lever presses during reinforcer delivery were recorded but produced no programmed consequence. The opposite wall of each chamber containing a response lever that was termed the inactive lever. Responses on the inactive lever were recorded but produced no programmed consequence. Behavior on this lever serves as a control for motor disruptions and other nonspecific effects of test compounds.

### Operant Alcohol Self-Administration: Training and Baseline

Alcohol self-administration training was conducted using general methods as previously reported and validated for evaluating mechanisms of the reinforcing effects alcohol (Schroeder J P, Overstreet D H, Hodge C W. The mGluR5 antagonist MPEP decreases operant ethanol self-administration during maintenance and after repeated alcohol deprivations in alcohol-preferring (P) rats. Psychopharmacology (2005) 179:262-270). Rats were first given 1-week exposure to the alcohol solution (ethanol 8% v/v+sucrose 2% w/v) vs. water in drinking bottles in their home cage. This provides habituation to a novel taste and initial experience with the drug. Then, during 3 initial training days, rats were placed in the self-administration chambers for a 16-h session to establish reliable lever pressing behavior. We have shown that this auto-shaping method, which relies on exploratory behavior, produces robust alcohol-reinforced lever press responding in rodents. During training and throughout the experiment each lever press response produced the alcohol solution on a fixed-ratio 1 (FR-1) schedule of reinforcement. After initial training, rats were allowed to voluntarily self-administer the alcohol solution during daily (M-F) 30-min sessions for 30 days to establish a stable longitudinal baseline.

### Operant Alcohol Self-Administration: Drug Testing

Testing the efficacy of Compound 1 to reduce the reinforcing, or rewarding, effects of alcohol. Following establishment of stable baseline self-administration behavior, effects of pre-session treatment with the novel mGluR5 negative allosteric modulator Compound 1 (0, 0.3, 1, 3, and 10 mg/kg, IP) were evaluated on operant alcohol self-administration behavior (n=12 rats). To control for potential handling or injection effects, rats (n=12) were first administered 3 habituation injections of vehicle (10% w/v kolliphor in BCD (10%)) prior to evaluating the Compound 1 dose response curve. Compound 1 (0-10 mg/kg) doses were administered according to a within-subject randomized Latin-Square dosing design to control for potential dose order effects. All doses were administered 1-h prior to the beginning of operant self-administration sessions based on known receptor occupancy data in rats following systemic injection. Drug testing was conducted on Tuesday and Friday of each week only if behavior remained stable (as compared to baseline) on intervening non-drug days. Parameters of operant performance were measured, including total lever press responses, number of headpokes into the liquid receptacle, and rate of operant responding, which is a direct measure of positive reinforcement function.

Efficacy comparisons to MPEP and Naltrexone. After completion of the acute Compound 1 dose response curve, effects of the mGluR5 antagonist MPEP (3 and 10 mg/kg, IP) and the opiate antagonist naltrexone (3 mg/kg, IP) were evaluated in the same rats for comparison of Compound 1 to compounds known to reduce operant alcohol self-administration. MPEP has a similar mechanism of action via mGluR5 and, importantly, naltrexone is FDA approved for treatment of AUD in humans; thus, providing comparison data to a validated therapeutic compound. Effects of MPEP were evaluated first followed by naltrexone. Like Compound 1 testing, each drug was administered 1-h prior to behavioral sessions and only on Tuesday or Friday under stable baseline conditions. The same vehicle (10% w/v kolliphor in BCD (10%) was used for all test compounds.

Repeated treatment with Compound 1. To evaluate effects of repeated treatment with Compound 1 on operant alcohol self-administration, a separate group of rats (n=12) was trained to self-administer alcohol as described above. After operant behavior reached a stable baseline, habituation to i.p. injections were conducted as described above. During habituation to i.p. injections, rats exhibited a severe stress response to injections and the study was terminated. Another group of rats (n=12) was then trained to self-administer alcohol and a new injection protocol (e.g., 2-hr pretreatment time) was initiated. This method also resulted in a stress response and reduced alcohol self-administration. In a final attempt to evaluate repeated treatment, we re-established baseline behavior in the same rats and administered Compound 1 (0 and 10 mg/kg) orally in a palatable food pellet as previously reported. Doses were administered according to an A-B-A (vehicle-Compound 1-vehicle) experimental design with 5 consecutive days of exposure to each vehicle treatment and 7 days of exposure to Compound 1. There was no observable stress response using this method of exposure. Operant self-administration (number of alcohol reinforced lever presses) was recorded during daily 15-min test sessions (n=12 rats).

### Cue-Induced Reinstatement of Alcohol-Seeking Behavior

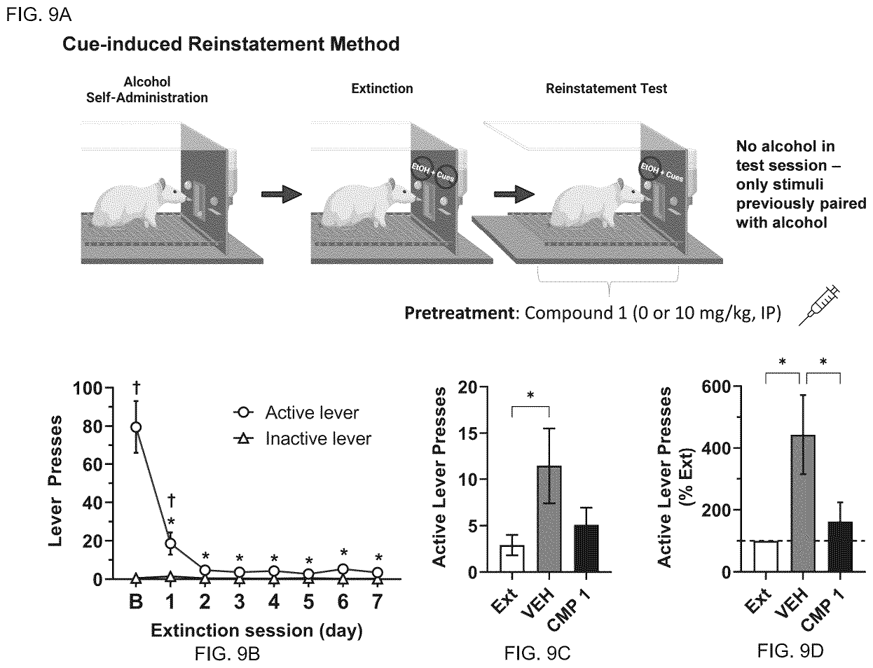

Operant alcohol self-administration training. Male Wistar rats (n=12) were trained to self-administer alcohol in two-lever operant conditioning chambers as described above. A total of n=11 rats completed the experiment. One rat failed to acquire the initial behavior. During all operant self-administration sessions (training and 30-day baseline period), responses on the alcohol-paired lever resulted in the presentation of a compound stimulus consisting of a visual cue light located above the response lever, and the auditory stimulus of pump activation from a fixed spatial location (see FIG. 9A, left panel). Responses on the inactive lever produced no programmed consequences. Fluid cups were inspected at the end of each session to verify intake. Extinction. Following the 30-day baseline, self-administration behavior was extinguished by removing all programmed consequences of lever press responding (i.e., absence of cue-light, pump sound, and sweetened alcohol delivery (see FIG. 9A, middle panel). Extinction training continued for 7 days at the end of which response totals on the active lever declined from 80 responses per session to 3 responses, which was not different from totals on the inactive lever and is consistent with full extinction of alcohol-reinforced lever press responding.

Cue-induced reinstatement test. On the next day, rats were given a reinstatement test in which lever press responses were followed by presentation of the cue light and auditory pump sound (as during training) but in the absence of ethanol reinforcement to examine cue-induced alcohol-seeking behavior (see Cue-induced reinstatement method FIG. 9A, right panel). One hour prior to the reinstatement session rats were divided into 2 equal subgroups groups and administered Compound 1 (0 or 10 mg/kg, IP) to determine if Compound 1 alters cue-induced reinstatement of alcohol-seeking behavior as compared to vehicle control. Groups were matched on body weight, baseline response totals, and extinction performance prior to drug testing. After this test, rats were returned to baseline conditions. After re-establishment of baseline performance, rats underwent a second extinction procedure followed by a second reinstatement test in a cross-over design where rats that received vehicle during the first test were administered Compound 1 (10 mg/kg) and rats that previously received Compound 1 were administered vehicle.

### Behavioral Data Analysis

The principal behavioral parameter of operant ethanol self-administration was the number of lever presses. Self-administration and extinction data were analyzed separately by one- or two-way analysis of variance (ANOVA) with factors for drug dose, lever (active vs. inactive) and time where appropriate (GraphPad Software Inc., La Jolla, CA USA). Multiple comparisons were conducted with Šídák's or Dunnett's multiple comparisons tests where appropriate.

### Results

Research Objective 3.1a—Effects of Acute Compound 1 on Home-Cage Alcohol Drinking

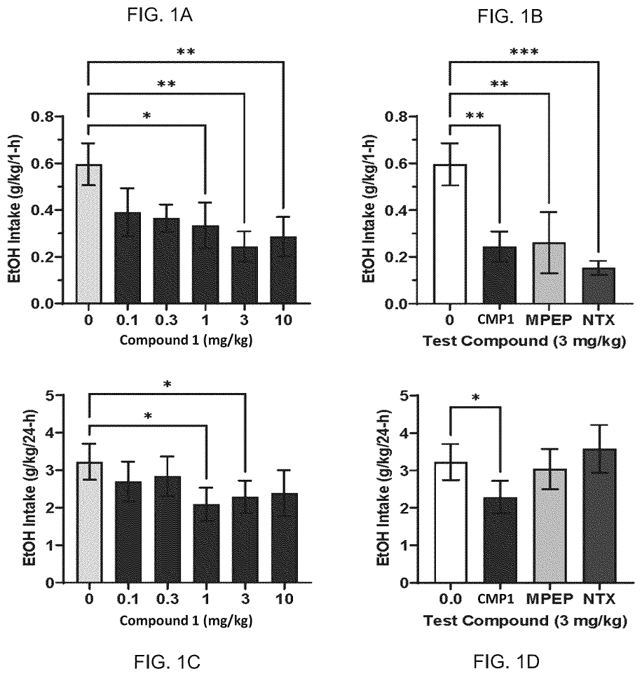

Alcohol intake (1-hr access). Rats consumed an average EtOH dosage of 0.596±0.09 g/kg (MEAN±SEM) during the first hour of access following vehicle pretreatment. Repeated measures analysis of variance (RM-ANOVA) showed that acute administration of Compound 1 (0-10 mg/kg) significantly decreased voluntary home-cage alcohol drinking by Wistar rats (F (5, 50)=3.040, P=0.0180). Follow up multiple comparison procedures showed that Compound 1 (1, 3, and 10 mg/kg) each significantly decreased alcohol intake as compared to vehicle control (FIG. 1A). The greatest reduction in EtOH intake was seen following the 3 mg/kg dosage of Compound 1. Effects of Compound 1 (3 mg/kg) were compared to MPEP (3 mg/kg) and Naltrexone (3 g/kg) as positive controls. RM-ANOVA showed that overall drug treatment significantly decreased alcohol drinking (F (3, 30)=7.74, P=0.0006). Multiple comparison procedures indicated that all three drugs significantly decreased alcohol drinking at 1-hr of access as compared to vehicle control (FIG. 1B). Thus, Compound 1 showed a high degree of efficacy for reducing voluntary alcohol drinking during the first hour of access when motivation in rodents is at a peak.

Alcohol intake (24-hr access). RM-ANOVA showed that acute pretreatment with Compound 1 (0-10 mg/kg) significantly reduced alcohol drinking measured after 24-hrs of access (F (5, 50)=2.429, P=0.0478). Follow up multiple comparison procedures showed that Compound 1 (1 and 3 mg/kg) each significantly decreased alcohol intake after 24-hrs of access (FIG. 1C). The trend toward reduced EtOH intake following Compound 1 (10 mg/kg) did not achieve statistical significance. Comparison of Compound 1 to equivalent doses of MPEP and Naltrexone showed that overall drug treatment significantly reduced alcohol intake at the 24-hr measurement interval (F (3, 30)=3.058, P=0.0433). Interestingly, follow up multiple comparisons showed that only Compound 1 (3 mg/kg) maintained efficacy at the 24-hr measurement interval (FIG. 1D) indicating improved efficacy of Compound 1 as compared to a similar acting compound (MPEP) and an FDA approved medication for treatment of AUD (Naltrexone). Results after 24-hr access show that Compound 1 (1 or 10 mg/kg) exhibits significant efficacy for reducing voluntary alcohol drinking over an extended period following treatment.

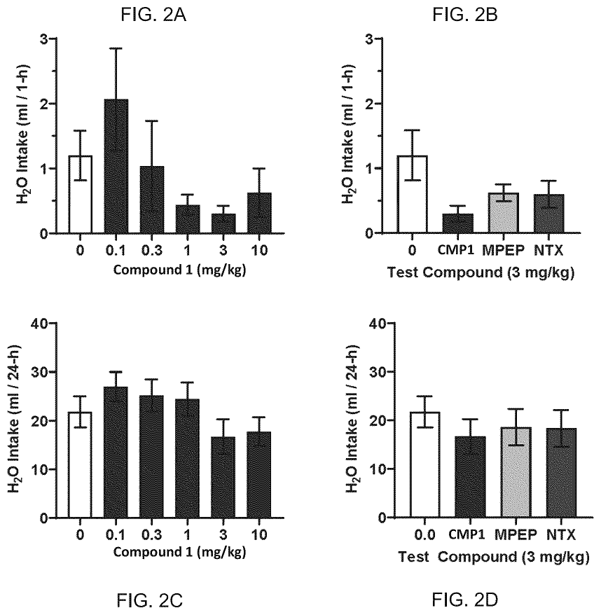

Water intake (1-hr and 24-hr access). RM-ANOVA showed that Compound 1 (0-10 mg/kg) or Compound 1 (3 mg/kg) in comparison to MPEP and Naltrexone. Trends toward reduced water intake did not reach statistical significance (FIG. 2A-2B). At the 24-hr measurement interval, RM-ANOVA identified an overall reduction in H₂O intake following pretreatment with the full dose range of Compound 1 (0-10 mg/kg) (F (5, 50)=6.542, P<0.0001). However, follow up multiple comparisons did not identify a significant change at any dose of Compound 1 as compared to vehicle (FIG. 2C). This suggests that overall trends (both slight increases and decreases) with little variability contributed to the main effect. Finally, there was no effect of Compound 1 (3 mg/kg) or comparison compounds on H₂O intake after 24-hr access (FIG. 2D). These results show a favorable profile indicating lack of nonspecific effect on general fluid consumption for Compound 1.

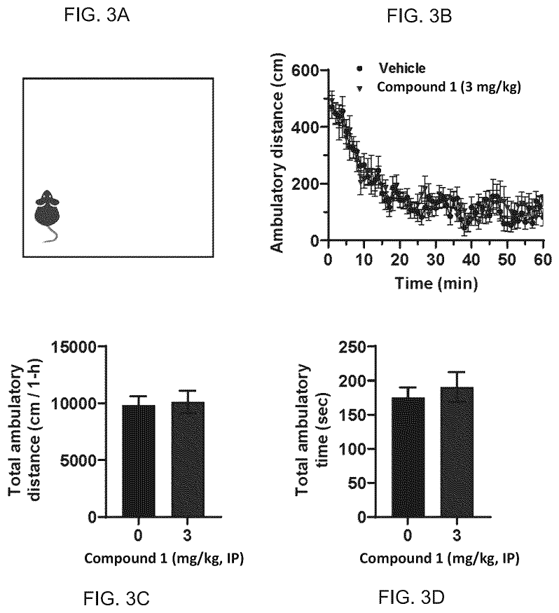

Open-field locomotor test for nonspecific behavioral effects. Pharmacological manipulations have the potential to alter rodent behavior by producing nonspecific effects on motor ability, learning and memory, or emotional processing. The open field test is a widely used method for assessing all these potential nonspecific effects in a single test. In this method, rodents are injected with a test compound and placed in an open-field apparatus that records ambulatory behavior in real time via computer analysis of photobeam breaks (FIG. 3A). Results showed that Compound 1 (3 mg/kg) had no effect on initial exploration or habituation to the novel open-field environment as compared to vehicle control (FIG. 3B). Similarly, Compound 1 (3 mg/kg) had no effect on summary measures of total ambulatory distance (FIG. 3C) or total ambulatory time (FIG. 3D) as compared to vehicle control. This indicates that normal learning, memory, and motor functions are intact and unaltered by drug treatment.

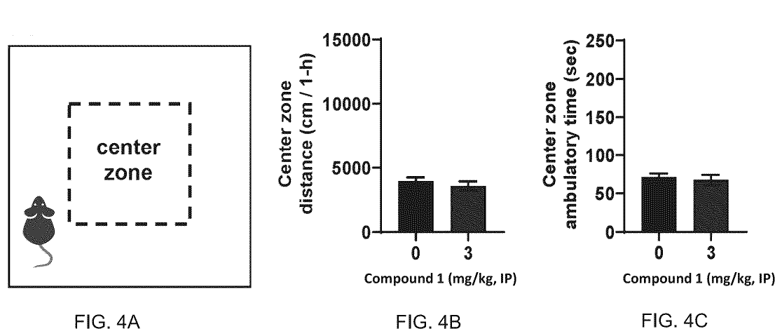

To evaluate potential nonspecific alterations in emotional processing, locomotor data were analyzed for potential alteration in motor activity in the center zone of the open field (FIG. 4A). Rats showed normal avoidance of the center zone following treatment with Compound 1 (3 mg/kg) as compared to vehicle (FIG. 4B and FIG. 4C). Overall, the important negative control results shown in FIG. 3 and FIG. 4 indicate that Compound 1 did not reduce alcohol drinking via nonspecific alterations in motor ability, cognitive function (spatial learning and memory) or emotional processing.

Research Objective 3.1b-Effects of Repeated Compound 1 on Home-Cage Alcohol Drinking

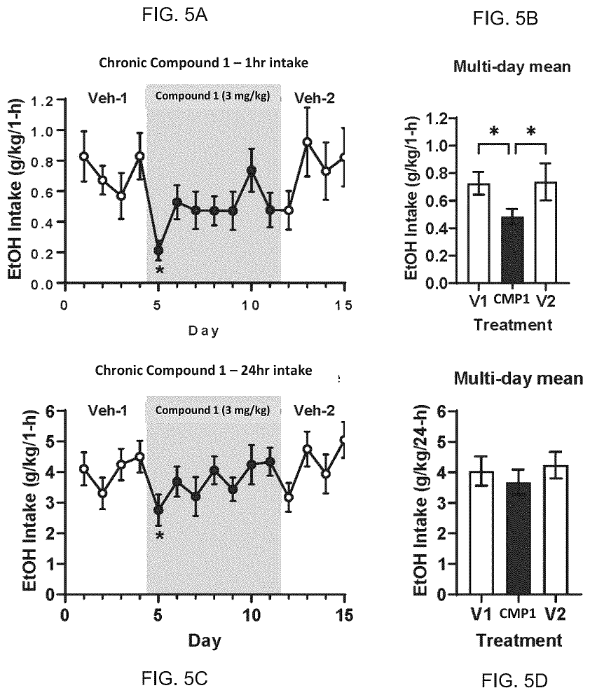

Alcohol intake (1-hr access). Rats consumed an average EtOH dosage of 0.73±0.08 g/kg (MEAN±SEM) during the first hour of access following vehicle pretreatment (Days 1-4; FIG. 5A). Repeated measures analysis of variance (RM-ANOVA) showed that repeated administration of Compound 1 (3 mg/kg) significantly decreased voluntary home-cage alcohol drinking by Wistar rats (F (14, 140)=2.303, P=0.0071). Follow up multiple comparison procedures of data form individual treatment days showed that Compound 1 (3 mg/kg) reduced alcohol intake only on day 5 as compared to the day 4 vehicle, although there was a trend toward reduced intake on days 2-5, and 7 (FIG. 5A). Alcohol drinking remained somewhat reduced on day 12 after return to vehicle administration, which suggests a residual effect of Compound 1 treatment. Subsequently, alcohol drinking returned to baseline levels on days 12-15 following vehicle treatment, suggesting that the effects of Compound 1 did not extend beyond day 12.

To further evaluate the effects of Compound 1 in the aggregate, daily alcohol drinking data from each condition (Veh-1, Compound 1, and Veh-2) were converted to an overall average of each rat's performance. RM-ANOVA showed that Compound 1 (3 mg/kg) significantly decreased alcohol intake (F (1.689, 16.89)=4.284, P=0.0366). Follow up multiple comparison procedures indicated that average alcohol intake during the 7-day Compound 1 treatment phase was lower that both Veh-1 and Veh-2 conditions (FIG. 5B). These results demonstrate preclinical efficacy of repeated Compound 1 treatment to reduce alcohol drinking during the first hour of access.

Alcohol intake (24-hr access). Rats consumed an average EtOH dosage of 4.04±0.48 g/kg (MEAN±SEM) after 2-hrs of access following vehicle pretreatment (Days 1-4; FIG. 5C). Repeated measures analysis of variance (RM-ANOVA) of the daily drinking data showed that repeated administration of Compound 1 (3 mg/kg) significantly reduced alcohol drinking (F (14, 140)=3.315, P=0.0001) as compared to average vehicle levels (FIG. 5C). Like the 1-hr data above, follow up multiple comparison procedures of data form individual treatment days showed that Compound 1 (3 mg/kg) reduced alcohol intake only on day 5 as compared to the day 4 vehicle (FIG. 5C). Visual inspection of the drinking data during Compound 1 treatment shows an increasing trend that is consistent with development of tolerance (FIG. 5C, middle panel). Also similar to the 1-hr results, alcohol drinking trended lower on day 12 but was not significantly different that initial vehicle days, via Dunnett's test. FIG. 5D shows no effect of Compound 1 (3 mg/kg) when data were averaged over each treatment conditions.

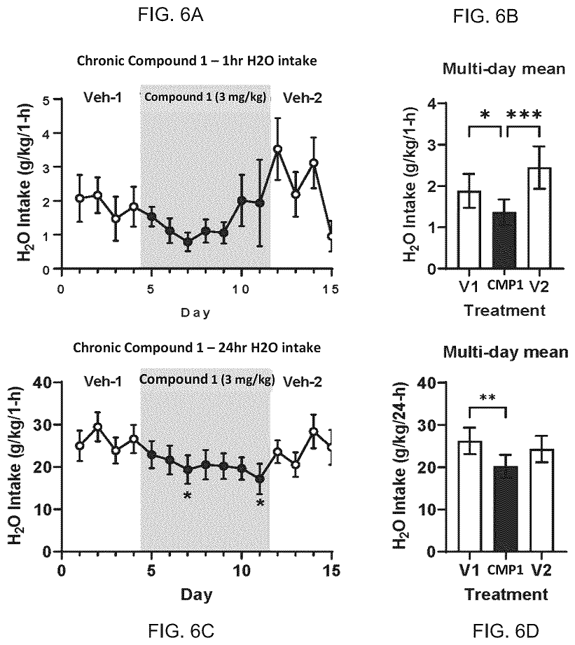

Water intake (1-hr and 24-hr access). RM-ANOVA showed that repeated treatment with Compound 1 (3 mg/kg) produced a significant overall reduction in H₂O intake after 1-hr of access as compared to vehicle controls (F (14, 140)=2.073, P=0.0167). However, multiple comparison procedures failed to identify a significant change at a particular treatment day (FIG. 6A). This result is likely due to nonsignificant trends in H₂O intake at various days. However, when evaluated by averaging H₂O intake across the 3 experimental phases, RM-ANOVA showed that Compound 1 decreased water intake F (2, 20)=9.959, P=0.0010) and that this overall effect was due to significant differences between the Compound 1 treatment phase and both vehicle control conditions (FIG. 6B). This analysis has increases statistical power due to making fewer post hoc comparisons and was more likely to detect a difference.

After 24-hrs access, RM-ANOVA detected an overall reduction in H₂O intake produced by Compound 1 (3 mg/kg) treatment (F (14, 140)=3.396, P<0.0001). Multiple comparison procedures showed that this overall effect was driven by significant reductions in H₂O intake at days 7 and 11 (FIG. 6C). This effect dissipated after return to vehicle administration during treatment days 12-15 (FIG. 6C). This effect on H₂O intake was also apparent when data were averaged across the experimental phases, RM-ANOVA: (F (1.824, 18.24)=7.370, P=0.0054). Holm-Šídák's multiple comparisons test found that the overall effect was driven by a Compound 1 mediated reduction in H₂O intake as compared to the first vehicle condition only (FIG. 6D). Overall, these reductions in H₂O intake were modest and had no effect on overall health as measured by experimenter and veterinary observations.

Research Objective 3.2.a—Acute and Repeated Dosing Strategy-Operant Self-Administration (Positive Reinforcement)

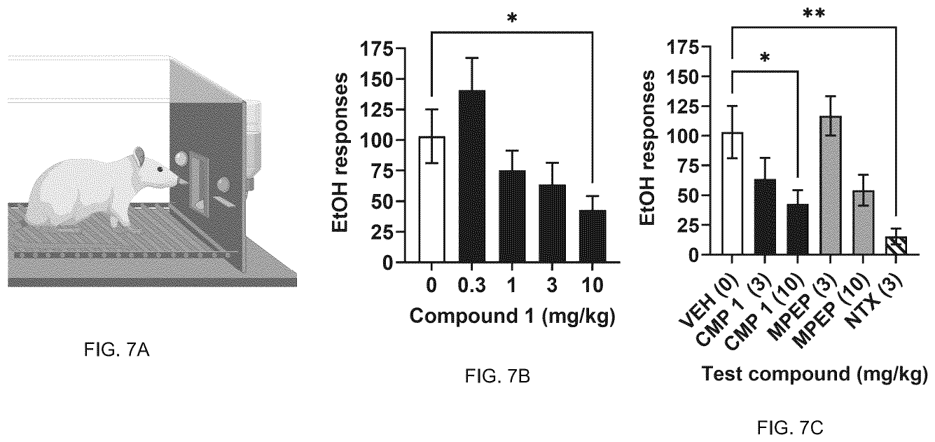

Effects of Acute Compound 1 on Operant Self-Administration. Rats were trained as described above to lever press for ethanol in operant conditioning chambers (FIG. 7A). Under baseline conditions, rats emitted an average of 103 responses on the ethanol lever. Compound 1 (0.3-10 g/kg, IP) pretreatment reduced alcohol reinforced lever press responses in a dose-dependent manner during 15-min test sessions [RM-ANOVA (F (2.917, 29.17)=3.810, P=0.0211)] (FIG. 7B). Dunnett's test showed that Compound 1 (10 mg/kg) significantly decreased alcohol intake after 24-hrs of access (FIG. 7B, asterisk). Comparison of Compound 1 (3 and 10 mg/kg) to equivalent doses of MPEP and Naltrexone (3 mg/kg) showed that Compound 1 (10 mg/kg) and naltrexone (3 mg/kg) exhibited similar efficacy to reduce alcohol reinforced lever press responses (FIG. 7C). Follow up multiple comparisons showed that Compound 1 (10 mg/kg) and Naltrexone (3 mg/kg) reduced EtOH reinforced responses (FIG. 7C, asterisks). Compound 1 (10 mg/kg) and Naltrexone (3 mg/kg) did not differ from one another, indicating equivalent efficacy.

IMPORTANTLY: These data indicate that: 1) Compound 1 dose-dependently reduced the positive reinforcing effects of alcohol; and 2) Compound 1 showed similar efficacy to FDA approved naltrexone in reducing alcohol reinforcement.

Effects of repeated treatment with Compound 1 on Operant Self-Administration. Rats (n=12) were trained to lever press for EtOH reinforcement as described above. After establishing stable baseline behavior and habituation to IP injections, daily injections of vehicle were conducted 1-h before operant self-administration sessions as part of an A-B-A (vehicle-Compound 1-vehicle) experimental design. Unfortunately, all rats in the group exhibited a major stress response to daily injections; thus, the study was halted.

A second group of rats (n=12) was purchased, and an alternative injection strategy (e.g., 2-h pre-session interval between injection and testing) was employed. Unfortunately, the new group of rats also showed a stress response to daily injections under the modified protocol.

In a final attempt to salvage the study, injections were halted in the second group of rats and EtOH self-administration behavior was allowed to stabilize over a 1-week period. Then, Compound 1 (0 or 10 mg/kg) was delivered orally via a palatable food pellet 2-hr before daily sessions according to the proposed A-B-A study protocol. The oral pellet method of drug delivery has been used successfully for activation of a TET-on viral vector, and models methods of oral drug delivery via palatable substances that are widely used in the field.

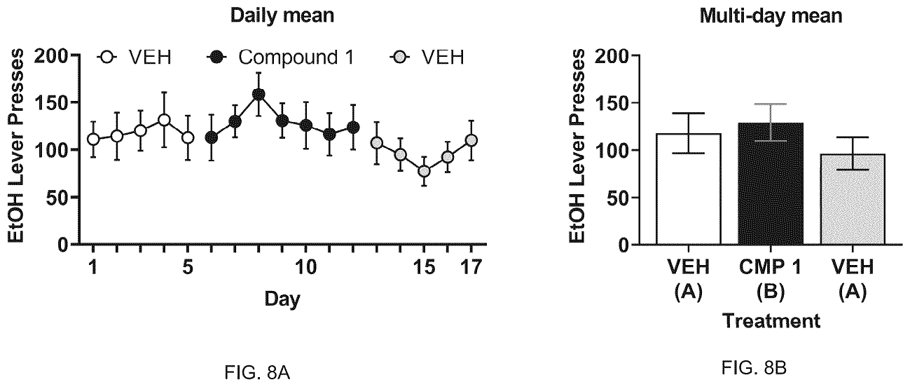

Under these conditions, rats (n=12) rapidly consumed the pellet containing Compound 1 (0 mg/kg, vehicle) and behavior remained stable for 5 days of testing. For the next 7 days, rats were administered Compound 1 (10 mg/kg, oral) in palatable food pellets followed by a return to vehicle for 5 days (A-B-A design). Daily results are illustrated in FIG. 8A, which shows that EtOH reinforced lever press responses were stable during the first vehicle exposure (Days 1-5). Administration of Compound 1 (10 mg/kg) failed to alter operant alcohol self-administration (Days 6-12). Return to vehicle (Days 13-17) in the final stage shows that behavior was somewhat variable as a function of day but exhibited no consistent upward or downward trajectory. FIG. 8B shows a summary plot where data from all rats were averaged across each experimental phase (A-B-A). Statistical analysis by one-way ANOVA found no significant effect of treatment on the multi-day mean of EtOH reinforced lever press responses F (2, 33)=0.7387, P=0.4855. Overall, this experiment did not show an effect of Compound 1 (10 mg/kg, oral pellet) on the reinforcing effects of alcohol. However, the apparent lack of effect should be interpreted with caution and may be due to pharmacokinetic and/or receptor occupancy differences between this oral consumption method and the more standard IP injection method, which produces a well-characterized profile of Compound 1 [other studies in this report and (Bennet K A, Sergeev E, MacSweeney CP, Bakker G and Cooper A E. Understanding exposure-receptor occupancy relationships for metabotropic glutamate receptor 5 negative allosteric modulators across a range of preclinical and clinical studies. The Journal of Pharmacology and Experimental Therapeutics (2021) 377:157-168.)]. Thus, full interpretation of the present results will require additional pharmacokinetic and behavioral testing.

### Research Objective 3.2.b.—Cue-Induced Reinstatement of Alcohol Seeking (Relapse)

Baseline self-administration. On the last day of baseline rats (n=11) responded significantly more on the EtOH (active) lever as compared to the concurrently available inactive lever (t (21)=5.781, P<0.0001) (FIG. 9B, baseline denoted by B on x-axis).

Extinction of alcohol self-administration. During the 7 days of extinction, responses on the lever that previously resulted in presentation of ethanol reinforcement and the environmental cues (stimulus light and pump sound) produced no programmed consequence. Overall, rats emitted more responses on the formerly active lever during extinction F (1, 21)=32.35, P<0.0001). However, consistent with extinction of behavior, responding on the formerly active lever decreased as a function of days (F (7, 147)=28.18, P<0.0001) and was not different from responding on the inactive lever after Day 1 (FIG. 9B). There was also a statistical interaction between time and lever (F (7, 147)=27.25, P<0.0001) indicating that the primary change in behavior was the time-dependent reduction in active lever responding. These data show extinction of ethanol reinforced responding (i.e., equivalent responding on levers that previously produced ethanol and the inactive lever) when all consequences of lever pressing were removed for 7 days.

Cue-induced reinstatement of alcohol-seeking behavior: blockade by Compound 1. On day 8 of the procedure, half of the rats were administered vehicle and the other half received Compound 1 (10 mg/kg) and then underwent reinstatement tests in which responses on the active lever resulted in presentation of discrete cues (stimulus light above the lever and pump sound) that were previously paired with ethanol delivery during baseline (FIG. 9A). Analysis of total response data via one-way RM-ANOVA showed a significant effect of treatment (vehicle vs Compound 1) condition (F (2, 20)=3.849, P=0.0385). Multiple comparison procedures showed that responding on the previously active lever was increased following vehicle treatment, which demonstrates cue-induced reinstatement, which was blocked by Compound 1 (FIG. 9C). Data were also analyzed relative to extinction performance using each rat as its own control to generate group means. These results showed a reinstatement effect of ˜400% of extinction level performance that was fully blocked by Compound 1 (F (2, 16)=6.146, P=0.0105) (FIG. 9D). Overall, the behavioral data demonstrate robust cue-induced reinstatement of alcohol-seeking behavior after vehicle treatment that was blocked by the mGluR5 inhibitor Compound 1 (FIG. 9C).

### CONCLUSIONS

Purpose of these studies: These studies were conducted to evaluate the preclinical efficacy of the mGluR5 negative allosteric modulator Compound 1 as a pharmacotherapy for AUD. We utilized rat behavioral methods that have exceptional translational relevance to the human disorder and address critical therapeutic indications. First, experiments determined if Compound 1 shows efficacy for reducing chronic alcohol drinking in the home-cage. Second, we utilized state-of-the-art operant alcohol self-administration methods (Schroeder J P, Overstreet D H, Hodge C W. The mGluR5 antagonist MPEP decreases operant ethanol self-administration during maintenance and after repeated alcohol deprivations in alcohol-preferring (P) rats. Psychopharmacology (2005) 179:262-270) to determine if Compound 1 shows efficacy to reduce the positive reinforcing effect of alcohol, which are a fundamental requirement of abuse liability (Stolerman, 1992). Third, we evaluated the potential efficacy of Compound 1 to reduce relapse-like behavior in rats. This was accomplished by evaluating the effects of pretreatment with Compound 1 on cue-induced reinstatement of alcohol-seeking behavior (Cannady R, Fisher K R, Durant B, Besheer J, Hodge C W. Enhanced AMPA receptor activity increases operant alcohol self-administration and cue-induced reinstatement. Addiction Biology (2013) 18:54-65.). This method utilizes operant conditioning to evaluate the ability of environmental stimuli previously paired with alcohol to trigger alcohol-seeking behavior, which models cue-induced craving that is common in humans suffering from AUD. The following sections provide a summary of results and translational conclusions related to these three preclinical domains.

### Therapeutic Indication: Reducing Chronic Alcohol Drinking

Results from Study 3.1a, showed that acute administration of Compound 1 (0-10 mg/kg) produced a dose-dependent reduction in home-cage alcohol intake, which is highly consistent with previously reported effects of FDA approved Naltrexone and Acamprosate in rat models of alcohol drinking. In this study, Compound 1 produced equivalent efficacy as compared to MPEP (a comparable mGluR5 inhibitor) and Naltrexone (FDA approved for treatment of AUD) when assessed after 1-hr of alcohol access. However, at 24-hr, only Compound 1 showed efficacy to reduce alcohol drinking, which indicates improved efficacy as compared to the FDA approved compound Naltrexone. The positive efficacy results obtained with Compound 1 occurred in the absence of nonspecific effects on water intake, motor activity, cognition, or anxiety-like behavior. This suggests that the impact of mGluR5 inhibition in active alcohol drinkers is relatively specific to alcohol use. Study 3.1b evaluated the effects of repeated Compound 1 (3 mg/kg) treatment on alcohol drinking. There was a robust effect on the first day of treatment with some loss of efficacy over successive treatment days. Translational Conclusion: Overall, these results indicate that Compound 1 shows significant preclinical efficacy for the reduction of chronic alcohol drinking using home-cage drinking methods that are standard in the field. Thus, Compound 1 may reduce chronic alcohol use in clinical populations with AUD.

Therapeutic Indication: Reducing the Positive Reinforcing Effects of Alcohol which Drive Repetitive Drug-Seeking Behavior

The fundamental behavioral process of reinforcement reflects the tendency of all animals, human and non-human, to repeat responses that produce a desired outcome. Accordingly, reinforcement mechanisms underlie the repetitive nature of alcohol seeking-behavior during both the initial binge/intoxication and subsequent dependence stages of addiction. Results from Study 3.2a, showed that acute administration of Compound 1 (0-10 mg/kg) produced a dose-dependent reduction in alcohol reinforced lever press responses. We compared the efficacy of Compound 1 to MPEP (a similar mGluR5 inhibitor) and Naltrexone (the opiate antagonist approved by the FDA for treatment of AUD). Compound 1 and Naltrexone demonstrated equivalent efficacy to reduce the positive reinforcing effects of alcohol. Study 3.2b attempted to evaluate the impact of repeated treatment with Compound 1; however, rats exhibited excessive stress responses to repeated IP injections and an alternative strategy for drug delivery (oral food pellet) was ineffective. Translational Conclusion: These results indicate that acute treatment with Compound 1 reduces the positive reinforcing effects of alcohol, which is expressed as a reduction in chronic operant alcohol self-administration. This finding has exceptional translational value because the reinforcing effects of alcohol are known to drive the chronic repetitive alcohol use that characterizes AUD. Thus, treatment with Compound 1 may be useful in the medical management of excessive chronic alcohol use in humans suffering from AUD.

### Therapeutic Indication: Reducing Cue-Induced Reinstatement of Alcohol-Seeking Behavior

Relapse to alcohol-seeking after abstinence is a hallmark behavioral pathology of AUD that is often triggered by conditioned reinforcement (cue-induced) processes. Clinical studies show that cues associated with alcohol use promote craving and relapse in abstinent alcoholics alcoholics. Thus, a key challenge for the field is identifying medications that block this behavioral process. In this study, rats were trained to self-administer alcohol in operant conditioning chambers. Each lever press response produced alcohol and a stimulus complex (stimulus light, pump sound). After establishing a stable chronic baseline of alcohol self-administration, extinction methods were conducted wherein lever press responses produced no programmed consequence-accordingly lever press responding on the active lever was extinguished to <4 responses per session. On a reinstatement test day, lever press responses produced the stimulus complex only (no alcohol) to evaluate the ability of cue exposure to trigger relapse-like behavior (e.g., an increase in non-reinforced lever pressing). Results showed that Compound 1 (10 mg/kg) significantly decreased cue-induced reinstatement of alcohol seeking behavior. Translational Conclusion: This finding indicates that treatment with Compound 1 blocks the ability of cues previously associated with alcohol use to trigger relapse-like behavior. Since exposure to environmental stimuli previously associate with alcohol use is a major cause of relapse in clinical AUD populations, this result strongly supports the potential value of Compound 1 as a medication for prevention of relapse in humans.

### Example 2—Evaluation of Compound 1 to Attenuate Cocaine Dependence Using the Conditioned Place Preference (CPP) Test in Rats

INTRODUCTION: Conditioned place preference (CPP) is a commonly used behavioral test to evaluate an animal's preference for an environment that has been associated with a positive reinforcer. Following CPP training, animals will spend more time in the environment associated with positive reinforcement compared to other environments without the positive reinforcer. This behavior can be used to evaluate the rewarding or aversive properties of a drug or stimulus.

OBJECTIVE: This study was designed to evaluate the efficacy of Compound 1 in attenuating cocaine dependence using the conditioned place preference (CPP) test in male SD rats.

### Material and Methods

Animals: Male Sprague Dawley rats (Envigo, IN, 275-299 g) were group housed upon arrival (2 per cage). All rats were examined and handled to assure adequate health and suitability. During the study, a 12/12 light/dark cycle was maintained (lights switched on at 7:00 am). The room temperature was maintained between 2° and 23° C. with a relative humidity maintained around 50%. Chow (LabDiet #5001) and water were provided ad libitum for the duration of the study. The test was performed during the animal's light cycle phase.

### Formulations:

Cocaine (10 mg/kg) was dissolved in saline and injected IP at a dose volume of 1 ml/kg. Cocaine was administered on training days 3, 5, 7 and 9 immediately prior to training.

3-((2-Methyl-4-thiazolyl)ethynyl)pyridine (MTEP, 3 mg/kg), was dissolved in saline and injected IP on day 10 (bias test) at a dose volume of 1 ml/kg 30 minutes prior to testing.

Compound 1 (1, 3 and 10 mg/kg) was formulated in 10% Solutol HS15 and 90% (10% aqueous 2-hydroxypropyl-β-cyclodextrin) and injected IP on day 10 (bias test) at a dose volume of 2 ml/kg. Compound 1 was administered 30 minutes prior to testing.

### Cpp Protocol:

The chamber used in this study contained two compartments [(60 cm (L)×40 cm (W)×24 cm (H)]. The compartment used for each animal and drug pairing was assigned and counterbalanced in advance and adjusted as necessary in the rebalancing procedure after day 1 (see details below). Within the chamber, visual and physical features were used to create the two distinct compartments. For example, one compartment may have a flat plastic floor with white stripes and the other may have a textured floor and black stripes. The chambers were cleaned with 70% alcohol in between runs.

Testing sessions were recorded on days 1 and 10 to determine time spent (seconds) in each chamber.

Day 1 Pre-conditioning/Habituation: Animals are allowed to explore the entire testing chamber freely for 20 min. Animals that display a strong preference for one compartment (>75% time spent in one compartment) are excluded.

Rebalancing Procedure: Animals are rebalanced following day 1 scoring if the difference between groups for time spent in one compartment is greater than 15%. Animals that failed to meet criteria (>75% time spent in one compartment) are replaced to complete balancing.

Days 2-9 Conditioning: All animals are treated with saline on days 2, 4, 6, and 8 and with cocaine or saline on days 3, 5, 7, and 9 depending on their assigned group. Animals are confined to their assigned compartment (saline- or cocaine-paired compartment) for 20 minutes following dosing.

Day 10 Post-conditioning bias test: Animals are dosed once with either saline, MTEP or Compound 1 (1, 3 and 10 mg/kg) according to their assigned. Following dosing, animals are allowed to explore the testing chamber freely for 20 minutes with the compartment dividing door open. Their activity exploring the chamber is recorded for analysis.

TABLE 1
CPP Procedure. Each session in 20 minutes.

| | | Alternation 1 | | Alternation 2 | | Alternation 3 | Alternation 4 | | | |
|---|---|---|---|---|---|---|---|---|---|---|
| | Day 1 | Day 2 | Day 3 | Day 4 | Day 5 | Day 6 | Day 7 | Day 8 | Day 9 | Day 10 |
| Saline Group | Habituation | Saline | Saline | Saline | Saline | Saline | Saline | Saline | Saline | Bias Test with |
| | | | | | | | | | | Saline |
| | | | | | | | | | | Treatment |
| Compound | Habituation | Saline | Cocaine | Saline | Cocaine | Saline | Cocaine | Saline | Cocaine | Bias Test with |
| Groups | | | | | | | | | | Compound |
| | | | | | | | | | | Treatment |

Statistical Analysis: Statistical analysis was conducted using GraphPad Prism 9. Scorers blinded to the treatment groups scored the amount of time each animal spent exploring each chamber on days 1 and 10. For bias test data, a one-way analysis of variance (ANOVA) followed by Dunnett's post hoc test was used. Significance was set at p<0.05 and values exceeding two standard deviations of the mean were considered statistical outliers and were excluded from the analysis. Additionally, if an animal spent >85% or <15% of the time in one compartment even without any obvious signs of sedation or locomotor dysfunction, they were excluded from the analysis. As a result of this criteria to ensure reliable data, the Compound 1, 3 mg/kg and 10 mg/kg groups have been excluded from the analysis due to low n-values, since half or more subjects had been removed from these two groups based on the criteria (see attached excel spreadsheet).

### Results

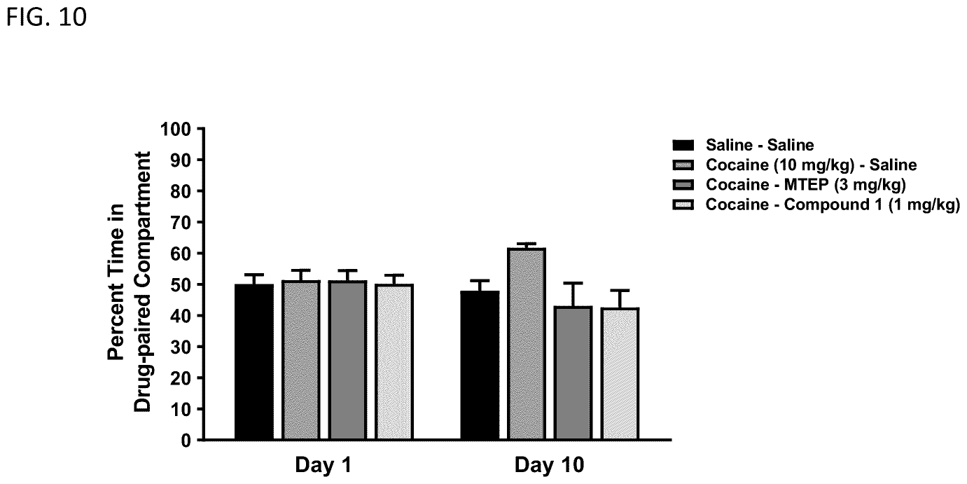

Percent of time in drug-paired compartment: The percentage of time spent in the drug-paired compartment on days 1 (baseline) and 10 (bias test) is shown in FIG. 10. On day 10, one-way ANOVA found a significant main effect of treatment [F (3,48)=4.708, p<0.01] with Dunnett's post hoc test showing a significant increase in time spent in the drug-paired compartment for the Cocaine-Saline group compared to the Saline-Saline group, indicating that Cocaine 10 mg/kg effectively induced CPP. Dunnett's test also showed that the Cocaine-MTEP and Cocaine-Compound 1 (1 mg/kg) groups spent significantly less time in the drug-paired compartment than the Cocaine-Saline group (p<0.05 and p<0.01, respectively), suggesting an attenuation of cocaine-induced CPP.

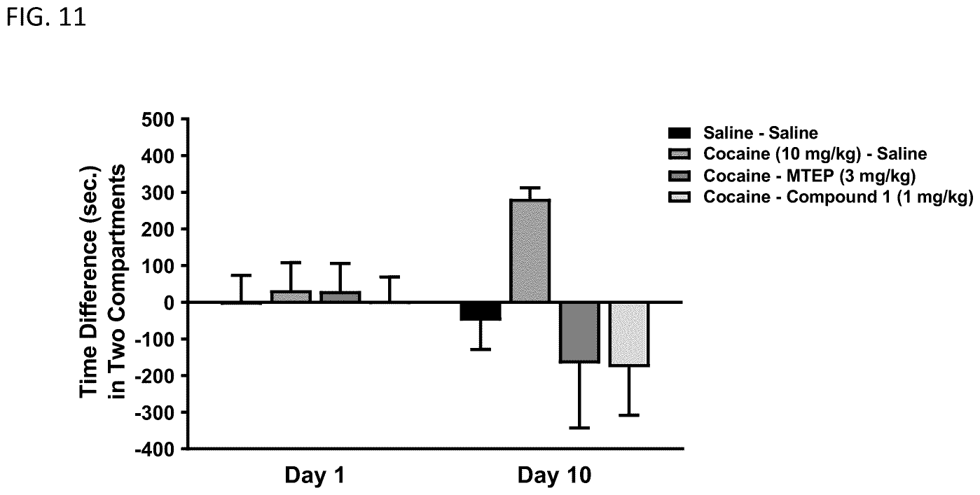

Preference scores: FIG. 11 shows the time difference between time spent in the drug-paired and the saline-paired compartment on days 1 (baseline) and 10 (bias test). On day 10, one-way ANOVA found a significant main effect of treatment [F (3,48)=4.709, p<0.01] with Dunnett's post hoc test showing a significant increase in time difference in the drug-paired compartment for the Cocaine-Saline group compared to the Saline-Saline group, indicating that Cocaine 10 mg/kg effectively induced CPP. Dunnett's test also showed that the Cocaine-MTEP and Cocaine-Compound 1 (1 mg/kg) groups had a significantly lower time difference than the Cocaine-Saline group (p<0.05 and p<0.01, respectively), suggesting an attenuation of cocaine-induced CPP.

### SUMMARY

The objective of this study was to evaluate the efficacy of Compound 1 in a CPP paradigm using cocaine as the reinforcer. Significant CPP was found in cocaine-treated group, indicating the validity of this assay in showing the abuse liability of cocaine. The results also showed that cocaine-induced CPP was attenuated by MTEP 3 mg/kg and Compound 1 (1 mg/kg) treatment, suggesting Compound 1 possesses potential in treating cocaine abuse.

Compound 1, 3 mg/kg and 10 mg/kg groups were excluded from data analysis because half or more subjects in these two groups showed extreme values (>85% or <15% of time spent in a single chamber), which is unlikely to happen in saline- or vehicle-treated rats. The extreme values occurred because those rats largely stayed in one compartment randomly. The side effects that occurred with Compound 1 (3 mg/kg and 10 mg/kg) doses may have obscured the compound's efficacy.

### Statistical Tables

TABLE 2
Percent time in compound-paired compartment

| ANOVA table | SS | DF | MS | F (DFn, DFd) | P value |
|---|---|---|---|---|---|
| Treatment (between | 3233 | 3 | 1078 | F (3, 48) = | P = |
| columns) | | | | 4.708 | 0.0058 |
| Residual (within | 10987 | 48 | 228.9 | | |
| columns) | | | | | |
| Total | 14220 | 51 | | | |

TABLE 3
Statistical Analysis for Percent time
in compound-paired compartment

| Dunnett's multiple | Mean | 95.00% Cl | Adjusted |
|---|---|---|---|
| comparisons test | Diff. | of diff. | P Value |
| Cocaine - Saline vs. Saline - Saline | 13.86 | 0.6152 to 27.11 | 0.0382 |
| Cocaine - Saline vs. Cocaine - MTEP | 18.73 | 3.687 to 33.78 | 0.011 |
| Cocaine - Saline vs. | 19.16 | 4.534 to 33.79 | 0.0071 |
| Cocaine - Compound 1 (1 mg/kg) | | | |

TABLE 4
Preference Scores

| ANOVA table | SS | DF | MS | F (DFn, DFd) | P value |
|---|---|---|---|---|---|
| Treatment (between | 1862261 | 3 | 620754 | F (3, 48) = | P = |
| columns) | | | | 4.709 | 0.0058 |
| Residual (within | 6327928 | 48 | 131832 | | |
| columns) | | | | | |
| Total | 8190189 | 51 | | | |

TABLE 5
Statistical Analysis for Preference Scores

| Dunnett's multiple | Mean | 95.00% Cl | Adjusted |
|---|---|---|---|
| comparisons test | Diff. | of diff. | P Value |
| Cocaine - Saline vs. | 332.9 | 15.02 to 650.8 | 0.038 |
| Saline - Saline | | | |
| Cocaine - Saline vs. | 449.6 | 88.52 to 810.7 | 0.011 |
| Cocaine - MTEP | | | |
| Cocaine - Saline vs. | 459.9 | 108.8 to 811.0 | 0.0071 |
| Cocaine - Compound 1 | | | |
| (1 mg/kg) | | | |

### Example 3—the Evaluation Compound 1 in Attenuating Cocaine Dependence Using the Intravenous Self-Administration (SA) Models in Rats

INTRODUCTION: Cocaine abuse represents a significant health problem worldwide. Cocaine rehabilitation has proven to be very challenging, not only because of the addictive properties of cocaine, but also because of the likelihood of relapse after a period of abstinence. Several pre-clinical models have been developed to assess compounds' abuse liability and/or evaluate their efficacy in treating drug dependence. Intravenous self-administration is widely accepted as a valid tool in this field. Using different protocols under this model, one can assess whether a novel compound shows efficacy in attenuating cocaine self-administration; one can also evaluate the efficacy of a novel compound in preventing or decreasing reinstatement of drug-seeking behavior after extinction of cocaine self-administration.

OBJECTIVE: The aim of this study was to evaluate the efficacy of Compound 1 on cocaine self-administration in rats.

### Material and Methods

Animals: Adult male Sprague-Dawley rats (300-325 g at arrival) from Envigo (Indiana, USA) were used. Upon arrival, rats were single housed in standard cages with filter tops and they were acclimated for at least 7 days before any treatment. All rats were examined and handled prior to initiation of the study. During the course of the study, 12 h/12 h light/dark cycles were maintained. The room temperature was 21-23° C. with a relative humidity maintained at 30-70%. Water was provided ad libitum for the duration of the study.

Test compounds: Cocaine Hydrochloride (Sigma-Aldrich, USA) was dissolved in saline (0.9% NaCl). In more detail, cocaine was made 1.05 mg/ml which is equivalent to 0.3 mg/kg/infusion per 350 g rats. The infusion rate was 0.1 ml/infusion.

Compound 1 was formulated in 10% Solutol HS15+90% (10% aqueous hydroxypropyl) betacyclodextrin. Compound was IP dosed 30 min before testing. The dose volume of Compound 1 was 2 ml/kg. Initially, for the fixed ratio attenuation test (Stage I), three doses 1, 3 and 10 mg/kg, were used. For the subsequent reinstatement test (Stage II), 3 mg/kg Compound 1 was used. During the 30 min treatment period, slightly decreased muscle tone was observed, but the rats were not sedated.

Reference compound MTEP (3-((2-Methyl-4-thiazolyl)ethynyl)pyridine, a selective allosteric antagonist of mGluR5) at 3 mg/kg was prepared with saline and IP dosed at 1 ml/kg dose volume.

Apparatus: Intravenous drug self-administration took place in experimental chambers within sound-attenuating cubicles equipped with an exhaust fan (Med Associates, VT). Each chamber contains two response levers situated on one wall of the chamber. A stimulus light was located above each lever and a house light was located at the top of the opposite wall. A pellet receptacle was situated between the two levers for delivery of food pellets (Bio-Serv's Dustless Precision Pellets #F0165, 45 mg). An infusion pump mounted above each chamber delivers drug solutions via Tygon tubing connected to a single channel fluid swivel, which was mounted on a balance arm above the operant chamber. The output of the liquid swivel was attached to the externalized terminus of the intravenous catheter.

Surgery and catheter maintenance: The rats were first implanted with jugular vein catheters (Access Technologies, USA). After the surgery, catheters were flushed daily with 0.2 ml of heparin-Enrofloxacin solution to avoid clogging and to ensure smooth drug infusion. The flushing liquid was made in 50 ml volume units which contained 1500 U Heparin and 320 mg Enrofloxacin (Baytril®). The solution was stored in sterilized vials in a 4° C. refrigerator. After the rats recovered from catheterization surgery, they were food-restricted (about 15 or 20 g/day) and maintain at ˜85% of their free-feeding, age-matched control body weight throughout the study. Starting from the 3rd day of food restriction, rats were trained to press the active lever (one of the two levers) to obtain food.

During the study, Methohexital sodium (Brevital®, Henry Schein Animal Health, USA) was used twice for catheter evaluation and thereby for indirect infusion confirmation. Brevital is a short-acting barbiturate that, when infused through the catheter, produces overt signs of sedation within seconds. Animals that do not show immediate signs of sedation suggest a dysfunction of their catheters, and they were removed from the experiment. In the present study, Brevital® test at the end of the acquisition training indicated that 23 out of 24 rats had functioning catheters.

Acquisition of cocaine self-administration: It took about 10 days for all the rats to reach criteria in food training (50 food pellets taken in a 1-hr session) thereby to be ready for the drug training. Rats were then allowed to self-administer cocaine solutions by pressing the active lever in a fixed-ratio (FR) schedule of reinforcement. In this study we used FR5, i.e., five lever presses for one drug infusion. Each drug infusion lasted 1.0 sec. One drug infusion was followed by a 20 second timeout period, during which time no drug was delivered even if the active lever was pressed. During the timeout period, the stimulus light above the active lever was on. Each training or testing session lasted 1 hour.

### Stage I: The Evaluation of Compound 1 Using Conventional Fixed Ratio Protocol

After establishing stable cocaine self-administration baseline (less than 20% variation in daily amount of drug infusions over 3 consecutive days; a minimum of 6 drug infusions per session) through 20 days of acquisition training, the rats were ready for compound testing. Compound testing was conducted twice a week (typically Tuesday and Friday), and baseline cocaine training was maintained on other days. Since 23 rats are much more than necessary (12 rats can provide reliable data), we randomly picked 16 rats for Stage I compound testing. The unchosen 7 rats continued to undergo daily cocaine training.

A within-subject design in which each rat received all treatments was applied with a Latin square test schedule. There were 6 treatment groups in this study: (1) Saline; (2) MTEP 3 mg/kg; (3) Compound vehicle; (4) Compound 1 (1 mg/kg); (5) Compound 1 (3 mg/kg); (6) Compound 1 (10 mg/kg).

### Stage II: The Evaluation of Compound 1 Using Reinstatement Protocol

After completion of Stage I, the rats were re-trained with cocaine for one week to ensure stable baseline. And then the rats underwent a 6-day extinction training procedure in which only saline infusion occurred when active lever was pressed. Meanwhile, the rats were divided into 3 roughly even groups for pharmacological stressor-induced reinstatement—All rats received one injection of alpha-2 adrenergic antagonist yohimbine (2 mg/kg) and then placed into the training box immediately). Thirty min before yohimbine treatment, one group of rats received saline, another group received MTEP 3 mg/kg injection, and the 3rd group received Compound 1 at 3 mg/kg.

Yohimbine-induced reinstatement was re-tested two more times in the same rats 3 days and 6 days after the first test, under continuous extinction procedure (There were 2 more days of extinction training between the 1st and 2nd reinstatement test, and between the 2nd and 3rd reinstatement test). Using the 3×3 within subject design, each given animal goes through all three treatments (saline, MTEP and Compound 1) across the three rounds of test.

Data Analysis: Prism software package Version 9 was used for statistical analysis and graphing. The number of active lever-press responses were recorded during each session and compared between treatment groups.

A one-way mixed-effects model (REML) of ANOVA followed by Dunnett's post hoc tests were used to analyze efficacy of Compound 1 and MTEP on cocaine self-administration in stages I (The repeated measure ANOVA was not totally applicable due to a few outlier data points, which are removed), and one-way mixed-effects model (REML) of ANOVA followed by Tukey's post hoc tests were used in Stage II. An effect was considered significant if P<0.05. Data were represented as the mean and standard error to the mean (s.e.m).

Statistical outliers which exceeded mean +/− (2× standard deviation) of the group were removed from the analysis. Also, for reinstatement, the extinction time was shortened (from 9 days plus extension as option to 6 days). In the present study, 3 rounds of reinstatement tests were performed, and we wished to maintain the reinstatement power, thus we cut extinction duration. Accordingly, we loosened the extinction criterion from 6 or less infusions to 9 or less infusions during the last extinction session. With this criterion, 17 out of 21 rats were eligible in drug test in Stage II. This adjustment did not change data quality.

### Results

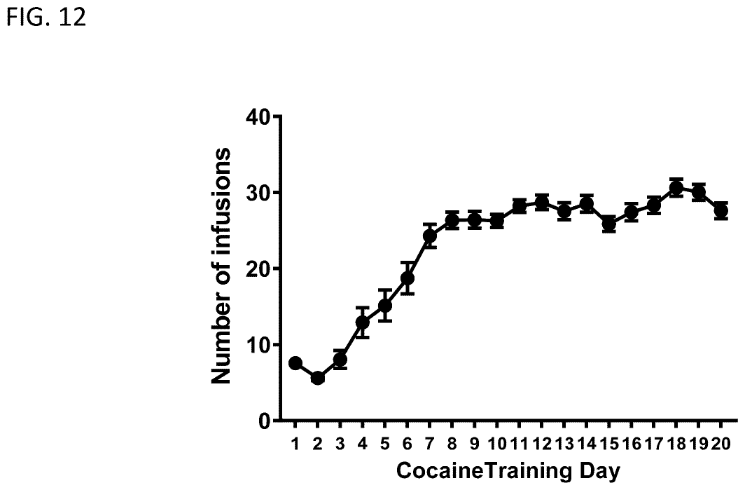

Cocaine acquisition: As shown in FIG. 12, The cocaine acquisition training lasted 20 days. The number of infusions increased steadily, and the curves had already reached a plateau after 15 days of acquisition training. Five more days of training were added to ensure sufficient acquisition.

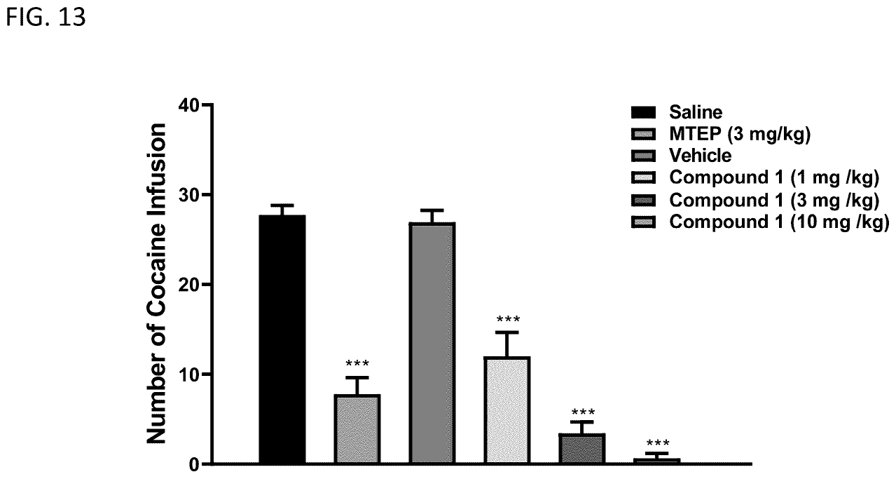

Compounds effects on cocaine self-administration: Compounds' effects on cocaine self-administration are illustrated in FIG. 13. A one-way mixed-effects model (REML) of ANOVA followed by Dunnett's post hoc tests (saline treatment as reference) found a significant main effect of treatment (F [2.525,35.86]=74.77, P<0.001). Post hoc comparisons showed that MTEP 3 mg/kg significantly decreased cocaine infusion. All three doses of Compound 1 also showed efficacy to significantly attenuate cocaine self-administration. The power of the compound progressively increased with higher doses.

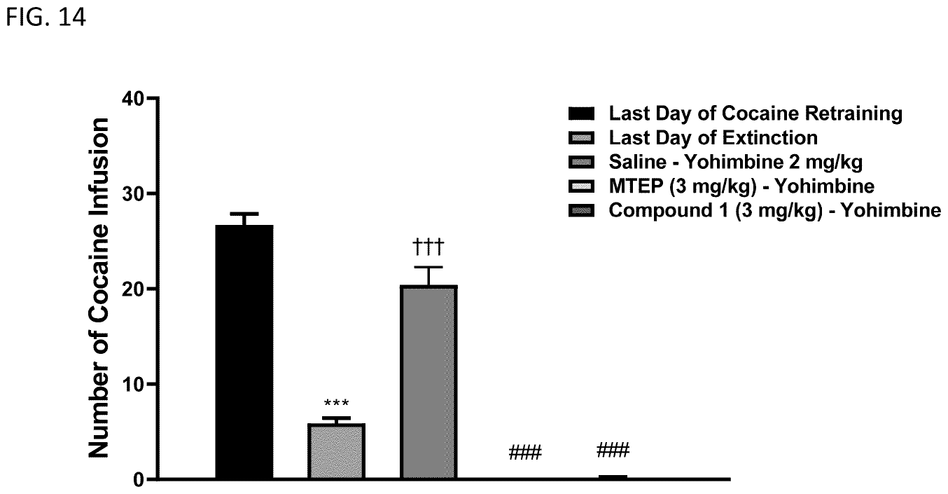

Compounds effects on pharmacological stressor-induced cocaine reinstatement: Based on the data from Stage I and signs of a potential side effect of Compound 1 at the 10 mg/kg dose (decreased muscle tone), a 3 mg/kg dose was chosen for Stage II. Compound 1's effects in the presence of a pharmacological stressor, yohimbine, induced reinstatement, a model for relapse (FIG. 14). One-way ANOVA with mixed-effects model (REML) found a significant effect of treatment (F [1.777,26.66]=150.7, P<0.001). Because multiple comparisons with different reference group are needed in this case, Tukey's post hoc test is used. Multiple comparisons showed that extinction training caused significant (˜80%) decrease of cocaine infusion (P<0.001). Yohimbine 2 mg/kg induced significant reinstatement (P<0.001). Both MTEP 3 mg/kg and Compound 1 (3 mg/kg) co-administered with yohimbine almost entirely prevented cocaine reinstatement (Ps<0.001).

### Summary

The present study tested the efficacy of test Compound 1 and reference compound MTEP in a rat model of cocaine self-administration.

The acquisition of cocaine self-administration was well established after 20 days of training. Using conventional FR5 protocol, MTEP 3 mg/kg and test Compound 1 at 1, 3 and 10 mg/kg all showed significant efficacy in attenuating cocaine infusion.

After a 6-day extinction of the self-administration response, the pharmacological stressor yohimbine 2 mg/kg led to significant reinstatement of cocaine self-administration (P<0.001). Both reference compound MTEP 3 mg/kg and Compound 1 (3 mg/kg) significantly prevented occurrence of the reinstatement (Ps<0.001).

This study demonstrates promising results in a rat model of cocaine self-administration, wherein Compound 1 effectively reduced cocaine infusions and prevented reinstatement of cocaine-seeking behavior in rats. These findings suggest that Compound 1 may have potential in treating cocaine abuse.

### Statistical Tables

### Stage I—Compounds Effects on Cocaine Self-Administration

TABLE 6
Stage I - Compounds effects on cocaine self-administration

| | Matching: | |
|---|---|---|
| Mixed-effects model (REML) | Across row | |
| Alpha | 0.05 | |
| Fixed effect (type III) | P value | F (DFn, DFd) |
| Treatment (between columns) | <0.0001 | F (2.525, 35.86) = 74.77 |
| Random effects | SD | Variance |
| Individual (between rows) | 3.598 | 12.94 |
| Residual | 5.249 | 27.55 |
| Data summary | | |
| Number of treatments (columns) | 6 | |
| Number of subjects (rows) | 16 | |
| Number of missing values | 4 | |

TABLE 7
Statistical Analysis for Stage I - Compounds
effects on cocaine self-administration

| Dunnett's multiple | Mean | 95.00% Cl | Adjusted |
|---|---|---|---|
| comparisons test | Diff. | of diff. | P Value |
| Saline vs. MTEP mg/kg | 19.93 | 15.86 to 24.00 | <0.0001 |
| Saline vs. Vehicle | 0.8 | −2.625 to 4.225 | 0.9375 |
| Saline vs. Compound 1 (1 mg/kg) | 15.73 | 9.481 to 21.99 | <0.0001 |
| Saline vs. Compound 1 (3 mg/kg) | 24.3 | 21.52 to 27.07 | <0.0001 |
| Saline vs. Compound 1 (10 mg/kg) | 27.07 | 24.37 to 29.77 | <0.0001 |

### Stage II: Compounds Effects on Cocaine Reinstatement:

TABLE 8
Stage II - Compounds effects on cocaine reinstatement

| | Matching: | |
|---|---|---|
| Mixed-effects model (REML) | Across row | |
| Alpha | 0.05 | |
| Fixed effect (type III) | P value | F (DFn, DFd) |
| Treatment (between columns) | <0.0001 | F (1.777, 26.66) = 150.7 |
| Data summary | | |
| Number of treatments (columns) | 5 | |
| Number of subjects (rows) | 17 | |
| Number of missing values | 4 | |

TABLE 9
Statistical Analysis for Stage II - Compounds effects on cocaine reinstatement

| | Mean | 95.00% Cl | Adjusted |
|---|---|---|---|
| Tukey's multiple comparisons test | Diff. | or diff. | P Value |
| Last Day of Cocaine Retraining vs. | 20.82 | 17.01 to 24.64 | <0.0001 |
| Last Day of Extinction | | | |
| Last Day of Cocaine Retraining vs. | 6.294 | 1.290 to 11.30 | 0.0106 |
| Saline - Yohimbine 2 mg/kg | | | |
| Last Day of Cocaine Retraining vs | 26.71 | 23.01 to 30.40 | <0.0001 |
| MTEP 3 mg/kg - Yohimbine | | | |
| Last Day of Cocaine Retraining vs | 26.57 | 22.74 to 30.41 | <0.0001 |
| Compound 1 (3 mg/kg) - Yohimbine | | | |
| Last Day of Extinction vs. | −14.53 | −20.13 to −8.931 | <0.0001 |
| Saline - Yohimbine 2 mg/kg | | | |
| Last Day of Extinction vs. | 5.882 | 4.052 to 7.712 | <0.0001 |
| MTEP 3 mg/kg - Yohimbine | | | |
| Last Day of Extinction vs. | 5.749 | 3.832 to 7.666 | <0.0001 |
| Compound 1 (3 mg/kg) - Yohimbine | | | |
| Saline - Yohimbine 2 mg/kg vs. | 20.41 | 14.02 to 26.80 | <0.0001 |
| MTEP 3 mg/kg - Yohimbine | | | |
| Saline - Yohimbine 2 mg/kg vs. | 20.28 | 13.90 to 26.66 | <0.0001 |
| Compound 1 (3 mg/kg) - Yohimbine | | | |
| MTEP 3 mg/kg - Yohimbine vs. | −0.1333 | −0.5680 to 0.3013 | 0.8655 |
| Compound 1 (3 mg/kg) - Yohimbine | | | |

### Example 4—Evaluation of the Efficacy of Compound 1 on Attenuating Oxycodone Dependence Using the Intravenous Self-Administration (SA) Model in Rats

INTRODUCTION: Opioid abuse represents a significant health problem worldwide. Opioid rehabilitation has proven to be very challenging, not only because of the addictive properties of cocaine, but also because of the likelihood of relapse after a period of abstinence. Several pre-clinical models have been developed to assess compounds' abuse liability and/or evaluate their efficacy in treating drug dependence. Intravenous self-administration is widely accepted as a valid tool in this field. Using different protocols under this model, one can assess whether a novel compound shows efficacy in attenuating cocaine self-administration; one can also evaluate the efficacy of a novel compound in preventing or decreasing reinstatement of drug-seeking behavior after extinction of opioid self-administration.

OBJECTIVE: The aim of this study was to evaluate the efficacy of Compound 1 on oxycodone self-administration in rats.

### Material and Methods

Animals. Adult male Sprague-Dawley rats (300-325 g at arrival) from Envigo (Indiana, USA) were used. Upon arrival, rats were single housed in standard cages with filter tops, and they were acclimated for at least 7 days before any treatment. All rats were examined and handled prior to initiation of the study. During the study, 12 h/12 h light/dark cycles were maintained. The room temperature was 21-23° C. with a relative humidity maintained at 30-70%. Water was provided ad libitum for the duration of the study.

Test Article(s)
Oxycodone (0.05 mg/kg/infusion; Sigma-Aldrich, USA) was dissolved in saline and infused at a rate of 0.1 ml/infusion.
Compound 1 (0.3, 1, and 3 mg/kg) was formulated in 10% Solutol HS15+90% (10% aqueous hydroxypropyl)-β-cyclodextrin and injected IP 30 minutes prior to test at a dose volume of 2 ml/kg. In the reinstatement study only 3 mg/kg was used.
MTEP (3-((2-Methyl-4-thiazolyl)ethynyl)pyridine; 3 mg/kg), was dissolved in saline and injected IP 30 minutes prior to testing at a dose volume of 2 ml/kg.

### Methods

Apparatus. Intravenous drug self-administration took place in experimental chambers within sound-attenuating cubicles equipped with an exhaust fan (Med Associates, VT). Each chamber contains two response levers situated on one wall of the chamber. A stimulus light was located above each lever and a house light was located at the top of the opposite wall. A pellet receptacle was situated between the two levers for delivery of food pellets (Bio-Serv's Dustless Precision Pellets #F0165, 45 mg). An infusion pump mounted above each chamber delivers drug solutions via Tygon tubing connected to a single channel fluid swivel, which was mounted on a balance arm above the operant chamber. The output of the liquid swivel was attached to the externalized terminus of the intravenous catheter.

Surgery and catheter maintenance: The rats were first implanted with jugular vein catheters (Access Technologies, USA). After the surgery, catheters were flushed daily with 0.2 ml of heparin-Enrofloxacin solution to avoid clogging and to ensure smooth drug infusion. The flushing liquid was made in 50 ml volume units which contained 1500 U Heparin and 320 mg Enrofloxacin (Baytril®). The solution was stored in sterilized vials in a 4° C. refrigerator. After the rats recovered from catheterization surgery, they were food-restricted (about 15 or 20 g/day) and maintained at ˜85% of their free-feeding, age-matched control body weight throughout the study. Starting from the 3rd day of food restriction, rats were trained to press the active lever (one of the two levers) to obtain food. During the study, Methohexital sodium (Brevital®, Henry Schein Animal Health, USA) was used twice for catheter evaluation and thereby for indirect infusion confirmation. Brevital is a short-acting barbiturate that, when infused through the catheter, produces overt signs of sedation within seconds. Animals without showing immediate signs of sedation suggest a dysfunction of their catheters, and they were removed from the experiment. The rats were first implanted with jugular vein catheters (Access Technologies, USA). After the surgery, catheters were flushed daily with 0.2 ml of heparin-Enrofloxacin solution to avoid clogging and to ensure smooth drug infusion. The flushing liquid was made in 50 ml volume units which contained 1500 U Heparin and 320 mg Enrofloxacin (Baytril®). The solution was stored in sterilized vials in a 4° C. refrigerator. After the rats recovered from catheterization surgery, they were food-restricted (about 15 or 20 g/day) and maintained at ˜85% of their free-feeding, age-matched control body weight throughout the study. Starting from the 3rd day of food restriction, rats were trained to press the active lever (one of the two levers) to obtain food. During the study, Methohexital sodium (Brevital®, Henry Schein Animal Health, USA) was used a few times for catheter evaluation and thereby for indirect infusion confirmation. Brevital is a short-acting barbiturate that, when infused through the catheter, produces overt signs of sedation within seconds. Animals that do not show immediate signs of sedation suggest a dysfunction of their catheters, and they were removed from the experiment. In the present study, Brevital® test at the end of the acquisition training a few more times indicated that 16 out of 20 rats had functioning catheters, and their data are eligible for analysis.

Acquisition of oxycodone self-administration. After reaching criteria in food training (50 food pellets taken in a 1-hr session) rats were allowed to self-administer oxycodone solutions by pressing the active lever in a fixed-ratio (FR) schedule of reinforcement. In this study we used FR3, i.e., three lever presses for one drug infusion. Each drug infusion lasted 1.0 sec. One drug infusion was followed by a 20 second timeout period, during which time no drug was delivered even if the active lever was pressed. During the timeout period, the stimulus light above the active lever was on. Each training or testing session lasted 1 hour.

Evaluation of Compound 1 on Oxycodone Self Administration. Testing commenced after establishing stable oxycodone self-administration baseline (less than 20% variation in daily amount of drug infusions over 3 consecutive days; a minimum of 6 drug infusions per session) through 20 days of acquisition training. Compound testing was conducted twice a week (typically Tuesday and Friday), and baseline oxycodone training was maintained on other days. Sixteen rats were used in the study. A within-subject design in which each rat received all treatments was applied with a Latin square test schedule. There were 6 treatment groups in this study: 1) Saline; 2) MTEP 3 mg/kg; 3) Compound vehicle; 4) Compound 1 (0.3 mg/kg); 5) Compound 1 (1 mg/kg); 6) Compound 1 (3 mg/kg).

Evaluation of Compound 1 on Oxycodone Reinstatement. After completion of testing rats were re-trained with oxycodone for one week to ensure stable baseline. Rats then underwent a 6-day extinction where only saline infusion occurred when active lever was pressed. Meanwhile, the rats were divided into 3 groups: All rats received one injection of alpha-2 adrenergic antagonist yohimbine (2 mg/kg) with 0 min pretreatment (rats were placed into the training box immediately). Thirty minutes before yohimbine treatment, one group of rats received saline, another group received MTEP 3 mg/kg injection, and the 3rd group received Compound 1 (3 mg/kg). Yohimbine-induced reinstatement was re-tested two more times in the same rats 3 days and 6 days after the first test, under continuous extinction procedure.

Statistical Analysis. Prism 9.40 software package Version 9 was used for statistical analysis and graphing. The number of active lever responses were recorded during each session and compared between treatment groups. A one-way mixed-effects model (REML) of ANOVA followed by Dunnett's post hoc tests were used to analyze efficacy of Compound 1 and MTEP on oxycodone self-administration in stages I (The repeated measure ANOVA was not totally applicable due to a few outlier data points, which are removed), and one-way mixed-effects model (REML) of ANOVA followed by Tukey's post hoc tests were used in Stage II. An effect was considered significant if P<0.05. Data were represented as the mean and standard error to the mean (s.e.m). Statistical outliers which exceeded mean +/− (2× standard deviation) of the group were removed from the analysis.

### Results

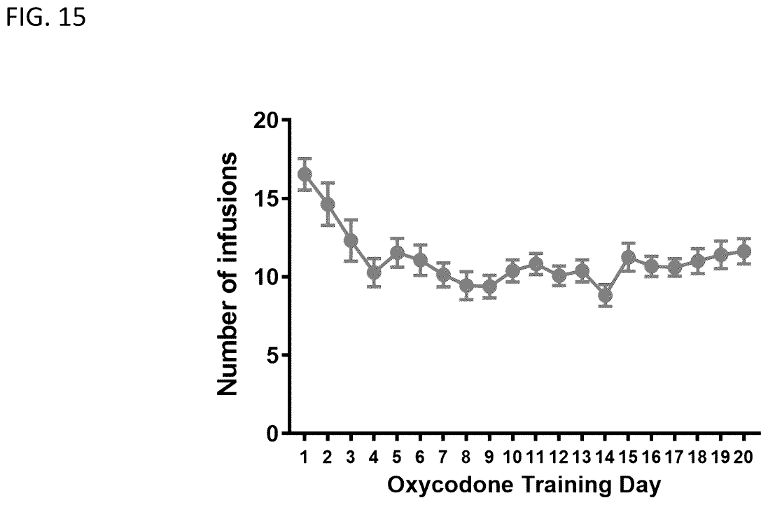

Oxycodone Acquisition. The oxycodone acquisition training lasted 20 days as shown in FIG. 15. The number of infusions slightly decreased from the momentum of food training in the first three days and maintained about 10-14 infusions/session on average until the training ended.

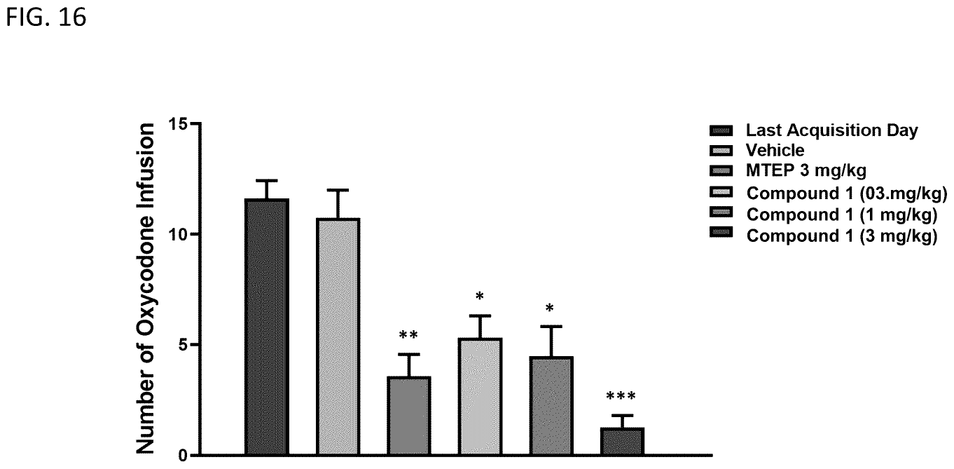

Effects of Compound 1 on Oxycodone Self Administration. The effect of Compound 1 on oxycodone self-administration is shown in FIG. 16. A one-way mixed-effects model (REML) of ANOVA followed by Dunnett's post hoc tests found a significant main effect of treatment (F [3.247,47.41]=17.73, P<0.001). Post hoc comparisons showed that MTEP 3 mg/kg significantly decreased self-administered oxycodone infusion compared to vehicle treatment. All three doses of Compound 1 also significantly attenuated oxycodone self-administration.

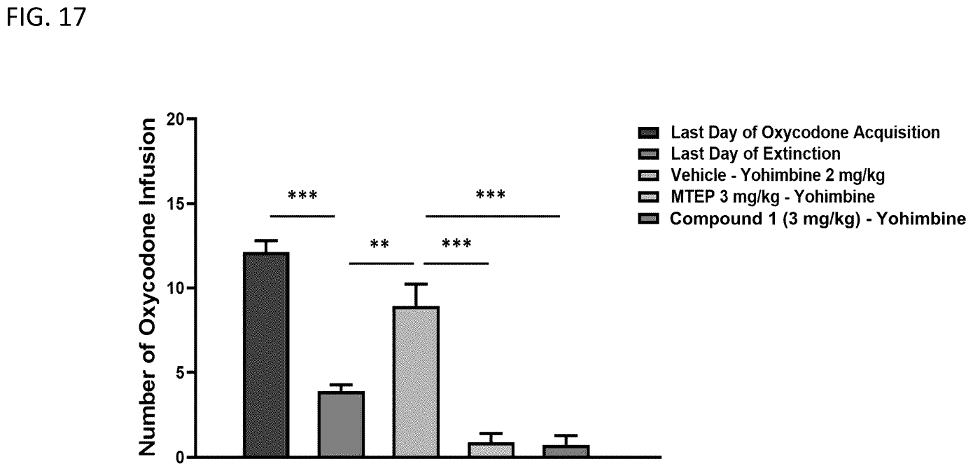

Effects of Compound 1 on Oxycodone Reinstatement. The effect of Compound 1 on yohimbine-induced reinstatement is shown in FIG. 17. One-way ANOVA with mixed-effects model (REML) found a significant effect of treatment (F [1.886,26.88]=55.96, P<0.001). Tukey's post hoc analysis showed that extinction training caused significant (˜70%) decrease of oxycodone infusion (P<0.001). Yohimbine 2 mg/kg induced significant reinstatement (P<0.001 relative to extinguished level). Both MTEP 3 mg/kg and Compound 1 (3 mg/kg) attenuated yohimbine-induced oxycodone reinstatement (Ps<0.001 relatively to Vehicle-Yohimbine group).

### Summary

The present study tested the efficacy of Compound 1 on attenuating oxycodone self-administration and reinstatement.

Acute injection of Compound 1 (0.3, 1 and 3 mg/kg) significantly decreased oxycodone self-administration. After extinction of the self-administration response, the pharmacological stressor yohimbine (2 mg/kg) led to significant reinstatement of oxycodone self-administration. Compound 1 (3 mg/kg) significantly prevented occurrence of the reinstatement. MTEP also attenuated oxycodone self-administration and reinstatement. These results suggest that Compound 1 possesses potential efficacy in treating oxycodone abuse.

### Statistical Tables

TABLE 10
Effect of Compound 1 on Oxycodone Self Administration.

| | Matching: | |
|---|---|---|
| Mixed-effects model (REML) | Across row | |
| Alpha | 0.05 | |
| Fixed effect (type III) | P value | F (DFn, DFd) |
| Treatment (between columns) | <0.0001 | F (2.525, 35.86 = 74.77 |
| Random effects | SD | Variance |
| Individual (between rows) | 3.598 | 12.94 |
| Residual | 5.249 | 27.55 |
| Data summary | | |
| Number of treatments (columns) | 6 | |
| Number of subjects (rows) | 16 | |
| Number of missing values | 4 | |

TABLE 11
Effect of Compound 1 on Oxycodone Reinstatement.

| Dunnett's multiple | Mean | 95.00% Cl | Adjusted |
|---|---|---|---|
| comparisons test | Diff. | of diff. | P Value |
| Vehicle vs. Last | −0.875 | −3.940 to 2.190 | 0.8849 |
| Acquisition Day | | | |
| Vehicle vs. MTEP | 7.188 | 2.476 to 11.90 | 0.0027 |
| 3 mg/kg | | | |
| Vehicle vs. Compound 1 | 5.438 | 09588 to 9.916 | 0.0154 |
| (0.3 mg/kg) | | | |
| Vehicle vs. Compound 1 | 6.283 | 1.135 to 11.43 | 0.0152 |
| (1 mg/kg) | | | |
| Vehicle vs. Compound 1 | 9.483 | 5.632 to 13.33 | <0.0001 |
| (3 mg/kg) | | | |

### Example 5—Phase 1, Randomized, Placebo Controlled, Multiple Ascending Dose (MAD) Study to Evaluate the Safety, Tolerability, and Pharmacokinetics of Compound 1 in Healthy Subjects

Example 5 presents the protocol for a suspended, multiple ascending dose (MAD) study to evaluate the safety, tolerability, and pharmacokinetics (PK) of Compound 1 in healthy subjects. In the following paragraphs, Example 5 is described in the future tense for sake of brevity; the study was suspended at day 9 after dosage of 6 patients. Greater than greater than expected exposure was observed at the 100 mg/day (i.e., 50 mg twice per day) dosage levels. Example 6 presents the protocol for a planned, amended Phase 1 MAD study.

Study Objectives and Endpoints—See Table 12 for Study Objectives and Endpoints. There will be up to 4 dose cohorts, and within each cohort subjects will be randomly assigned to receive either active drug or placebo. Study subjects will be administered active drug or placebo twice daily for 14 days (note: only the AM dose is administered on Day 14).

In this study, genetic samples will be collected for possible exploratory pharmacogenetics research that may be conducted to investigate the association between genetic factors (genotypes) and clinical assessments (phenotypes). If conducted, these studies would aim to better identify inherited genetic factors which may predict response to treatment with Compound 1, predict relative susceptibility to drug-drug interactions, predict genetic predisposition to side effects, or provide more information regarding Compound 1 in the disease state and how subjects may respond to Compound 1.

TABLE 12
Study Objectives and Endpoints

| OBJECTIVES | ENDPOINTS |
|---|---|
| Primary | Primary |
| To assess the safety and tolerability | The primary endpoint is safety assessed after |
| of Compound 1 following multiple | multiple-dose administrations by: |
| ascending oral doses | Adverse events (AE) |
| | Clinical laboratory tests |
| | Vital sign measurements |
| | 12-lead electrocardiograms (ECG) |
| | Physical examinations |
| | Psychiatric assessments (Brief Psychiatric |
| | Rating Scale [BPRS], |
| | Clinician-Administered Dissociative States |
| | Scale [CADSS], Columbia Suicide Severity |
| | Rating Scale [C-SSRS], Modified |
| | Mini-Mental State Examination (3MS) and |
| | Visual Analogue Scale [VAS]) |
| Secondary | Secondary |
| To assess the pharmacokinetics (PK) | The secondary objective will be assessed |
| profile of Compound 1 following | with the following endpoints: |
| multiple-ascending oral doses | Day 1: AUC from time zero to the time the |
| To evaluate the effect of a high-fat, | end of the 12-hr dosing interval |
| high-caloric meal on the steady-state PK | (AUC_{0-TAU, FD}), maximum concentration |
| of Compound 1 at a dose to be | (C_{max, FD}), time of maximum concentration |
| determined after the first cohort has | (T_{max, FD}), minimum concentration (C_{min, FD}), |
| completed | all after the first dose (FD) |
| | Day 13 (Fed State) and Day 14 (Fasting |
| | State): AUC_{0-TAU, SS}, C_{max, SS}, T_{max, SS}, and |
| | C_{min, SS}, all at steady-state (SS), along with |
| | apparent total plasma clearance (CL/F) and |
| | apparent volume of distribution (Vz/F). |
| Exploratory | Additional PK evaluations |
| To assess endpoints related to | Accumulation ratio for ARAUC derived as |
| Compound 1 mechanism of action | AUC_{TAU, SS}/AUC_{TAU, FD} and ARCMAX as |
| To evaluate CYP1A2 genotypes and | C_{max, SS}/C_{max, FD} |
| their association with phenotypes for | Estimations of dose proportionality at steady |
| potential impact on metabolism and to | state |
| identify biomarkers of potential | Estimation of time to achieve steady state |
| response to Compound 1. | Food effect- evaluation will compare the |
| | AUC_{TAU, SS} and C_{max, SS} at steady-state following |
| | a high-fat, high caloric meal and following |
| | fasted conditions at a single dose level |
| | (cohort to be determined) |

### Study Design

### Adaptive Features and Risk Management of Study Design

The rationale of having the following adaptive features is based on the hypothesis-forming approach of this clinical trial. The following categories will be adapted as follows:

TABLE 13
Adaptive Features and Boundaries

| Adaptive study | | |
|---|---|---|
| design category | Adaptive Features | Boundaries |
| Dose level | Doses for the subsequent | Doses are intended to escalate; |
| | cohorts will be | however, they may be |
| | determined/adapted based on | adjusted to an intermediate |
| | tolerability, and PK data (if | (lower), an equivalent dose or |
| | data permits) collected in | a higher dose as per the |
| | previous cohorts. | adaptive features. For any |
| | | individual subject the |
| | | maximum systemic exposure |
| | | should not exceed or be |
| | | predicted to exceed an AUC₀₋₂₄ |
| | | of 8100 h · ng/mL at steady |
| | | state and/or a maximum serum |
| | | concentration predicted to |
| | | exceed a Cₘₐₓ of 391 ng/mL. |
| The number of | The quantity of MAD cohorts | The maximal number of |
| MAD cohorts | may be adapted | different dose level cohorts |
| | | will be 4. |
| Dose Titration | An additional dose titration | Based on safety/tolerability |
| | cohort may be added in this | findings in any of the previous |
| | study based on interim safety | cohorts, a 14-day dose |
| | data reviews and tolerability | titration cohort (7 days |
| | to the study drug. | titration + 7 days stable |
| | | dosing) may be added. |

Abbreviations: MAD = multiple ascending dose; PK = Pharmacokinetics.

Decision-making process for the above adaptive study categories will be as follows: (1) Interim review of safety and tolerability of Compound 1 and emerging data from completed or ongoing MAD cohorts in a blinded fashion by the SRC. (2) Outcome on the adaptive study category will be documented by the SRC.

Based on the above, implementation can be performed without delay and Institutional Review Board (IRB) approval. If any adaptive features are outside the above pre-specified boundaries, IRB review and approval is mandatory before implementation.

### Maximum Tolerated Dose (MTD)

The Maximum Tolerated Dose (MTD) is defined as the highest dose that can be administered without clinically significant adverse events (AEs). The MTD will be determined by dose escalation until the stopping criteria are met as outlined in the section Stopping Rules.

The MTD is defined as the highest dose that does not cause unacceptable side effects. If no such toxicity is observed, the dose received in Cohort 3 will be considered the MTD.

### Overall Study Design

This study will be a randomized, double-blind, placebo-controlled, fixed-sequence, MAD study. The study will be conducted in a single clinical research unit (CRU). The study will consist of 3 cohorts, with the option of an additional cohort (intermediate dose cohorts or titration cohort). Each cohort will consist of 8 subjects (6:2 for active: placebo), for a maximum total sample size of approximately 32 subjects. Subjects will only participate in 1 cohort.

Screening will occur within approximately 28 days prior to the first scheduled study drug administration. Screening data will be reviewed to determine subject eligibility. Subjects who meet all inclusion criteria and none of the exclusion criteria and who consent to participation will be admitted to the CRU for baseline evaluations prior to dosing. All baseline safety results should be available prior to the first study drug administration.

Subjects will be fasted overnight for 10 hours prior to the morning dose, followed by a 2 hour fast. Subjects will fast for 2 hours prior to dosing and 2 hours following the evening dose. For Cohort 3, on Day 13 subjects will receive a standardized high-fat, high-calorie meal 30 minutes before dosing.

Each subject will be randomly assigned to 1 of the following cohorts: Cohort 1: Group 1 (6 subjects): 50 mg of Compound 1 twice daily (bid); Group 2 (2 subjects): placebo (matching Compound 1) bid. Cohort 2: Group 3 (6 subjects): 100 mg of Compound 1 bid; Group 4 (2 subjects): placebo (matching Compound 1) bid. Cohort 3: Group 5 (6 subjects): 150 mg of Compound 1 bid; Group 6 (2 subjects): placebo (matching Compound 1) bid.

In the event of low bioavailability in cohort 1 and cohort 2, resulting in a lower-than-expected predicted exposure in cohort 3 (C_{avg,ss}<100 ng/mL), the planned dosing schedule may be adjusted to allow administration of a 200 mg dose in cohort 3.

Safety will be assessed and blood samples for PK will be collected throughout confinement. Subjects will be discharged from the CRU on Day 18. Subjects will return to the CRU on Day 25 for a follow-up visit and End of Study procedures.

The maximum duration of subject participation, including screening, will be approximately 53 days.

Subjects who terminate the study early will perform follow-up procedures at the time of Early Termination.

Based on safety and tolerability findings in any of the cohorts, a 14-day dose titration cohort to evaluate the impact on TEAEs may be added as an additional cohort (7 days titration+7 days stable dosing).

The schedule of activities (SoA) of the study is described in Table 16.

### Study Treatments

The following treatments will be administered according to Table 14. (1) IP: Compound 1 oral tablet. (2) Placebo: Matching placebo

TABLE 14
Dose Cohorts

| | N | | |
|---|---|---|---|
| Cohort | (Active:Placebo) | Dose | Drug Administration |
| 1 | 6:2 | Twice daily | Twice daily Compound 1 |
| | | dose of 50 mg | or placebo dosing from |
| 2 | 6:2 | Twice daily | Days 1 to 13 and once |
| | | dose of 100 mg | in the morning on Day |
| 3 | 6:2 | Twice daily | 14 (for a total of 27 |
| | | dose of 150 mg | consecutive study drug |
| 4 | 6:2 | Titration or | administrations) |
| (Optional) | | Intermediate | |
| | | dose cohort | |

### Subject Population

Subjects meeting all the inclusion criteria and none of the exclusion criteria at screening may be eligible for participation in this study. Continued eligibility will be assessed upon admission to the clinical site, prior to the first study drug administration.

Screen failures are defined as participants who consent to participate in the clinical trial but are not subsequently randomly assigned to the study intervention or entered in the study.

### Inclusion Criteria

Provision of signed and dated informed consent form (ICF)

Stated willingness to comply with all study procedures and availability for the duration of the study

Healthy adult male or female

If male, meets one of the following criteria:
a) Is able to procreate and agrees to use one of the accepted contraceptive regimens and not to donate sperm from the first study drug administration to at least 90 days after the follow-up visit. An acceptable method of contraception includes one of the following: Abstinence from heterosexual intercourse; Male condom with spermicide or male condom with a vaginal spermicide (gel, foam, or suppository)
Or b) Is unable to procreate; defined as surgically sterile (i.e., has undergone a vasectomy at least 180 days prior to the first study drug administration)

If female, meets one of the following criteria:
(1) Physiological postmenopausal status, defined as the following: absence of menses for at least 12 months prior to the first study drug administration (without an alternative medical condition); and Follicle stimulating hormone (FSH) levels ≥40 mIU/mL at Screening;
Or (2) Surgical postmenopausal status, defined as the following: bilateral oophorectomy, salpingectomy, tubal ligation; and/or hysterectomy

Aged at least 18 years but not older than 59 years, inclusive, at the time of informed consent

Body mass index (BMI) within 18.5 kg/m2 to 32.0 kg/m2, inclusively

Minimum body weight of at least 50.0 kg

Non- or ex-smoker (An ex-smoker is defined as someone who completely stopped using nicotine products for at least 90 days prior to the first study drug administration)

Must be willing to abstain from drinking coffee or caffeine containing beverages during the study

Have no clinically significant diseases captured in the medical history or evidence of clinically significant findings on the physical examination (including vital signs) and/or ECG, as determined by an Investigator

Has clinical laboratory test results within the reference ranges of the testing laboratory, with the exception of results outside the reference ranges that are deemed not clinically significant by the Investigator (or designee) at Screening and check-in

Has supine blood pressure and pulse rate within the following ranges after 5 minutes rest: systolic blood pressure 90 to 140 mmHg, diastolic blood pressure 50 to 90 mmHg, and pulse rate 45 to 90 bpm at screening and on Day −1.

### Exclusion Criteria

Female who is lactating

Female who is pregnant according to the pregnancy test at Screening or prior to the first study drug administration

Female using the following systemic contraceptives: oral, patch or vaginal ring, in the 28 days prior to the first study drug administration

Female using hormone replacement therapy in the 28 days prior to the first study drug administration

Female using the following systemic contraceptives: injections or implant, or hormone-releasing intrauterine device in the 13 weeks prior to the first study drug administration and during the study

Drinking excessive amounts of tea, coffee, chocolate, and/or beverage containing caffeine (>2 cups/day)

Use of tobacco or nicotine containing products (including but not limited to; cigarettes, electronic cigarettes, pipes, cigars, chewing tobacco, nicotine patch, or nicotine gum) within 90 days prior to the first study drug administration and the inability to abstain from nicotine containing products until the follow-up visit.

Past or current history of any mental, behavioral, or neurodevelopmental disorder as defined by the tenth revision of the International Classification of Diseases (ICD-10). Subjects with family history of significant mental, behavioral, or neurodevelopmental disorders unless determined by the Investigator (or designee) and agreed by the Medical Monitor to be non-clinically significant (NCS) will be excluded

History or clinical manifestation of any metabolic, allergic, dermatological, hepatic, renal, hematological, pulmonary, gastrointestinal, neurological, respiratory, or endocrine disorder, unless determined by the Investigator (or designee) and agreed by the Medical Monitor to be NCS

Active or history of cardiovascular or cerebrovascular disease, including hypertension, angina, ischemic heart disease, transient ischemic attacks, bundle branch block, evidence of myocardial ischemia, stroke, and peripheral arterial disease sufficient to cause symptoms and/or require therapy to maintain stable status

History of significant hypersensitivity, intolerance, or allergy to any drug compound, food, or other substance, unless approved by the Investigator (or designee)

Active neoplastic disease or history of any neoplastic disease within 5 years of Screening (except for basal or squamous cell carcinoma of the skin or carcinoma in situ that has been definitely treated with standard of care)

Active infection (e.g., sepsis, pneumonia, abscess) or a serious infection (e.g., resulting in hospitalization or requiring parenteral antibiotic treatment) within 6 weeks prior to dosing

History of stomach or intestinal surgery or resection that would potentially alter absorption and/or excretion of orally administered drugs (uncomplicated appendectomy and hernia repair will be allowed)

Any of the following at Screening and/or prior to the first study drug administration:

QT interval corrected for heart rate using Fridericia's method (QTcF), QRS duration, PR interval outside of normal limits confirmed by repeat measurement, unless deemed NCS by PI and agreed by medical monitor;

Findings which would make QTc measurements difficult or QTc data uninterpretable;

History of additional risk factors for torsades de pointe (e.g., heart failure, hypokalemia, family history of long QT syndrome);

Maintenance therapy with any drug or significant history of drug dependency or alcohol abuse (>3 units of alcohol per day, intake of excessive alcohol, acute or chronic);

Positive test result for alcohol, cotinine, and/or drugs of abuse at Screening or prior to the first drug administration;

Positive screening results to HIV Ag/Ab combo, hepatitis B surface antigen or hepatitis C virus tests;

And any other clinically significant abnormalities in laboratory test results at Screening that would, in the opinion of an Investigator, increase the subject's risk of participation, jeopardize complete participation in the study, or compromise interpretation of study data;

Intake of an IP in the 28 days prior to the first study drug administration

Use of any prescription drugs in the 28 days prior to the first study drug administration, that in the opinion of an investigator would put into question the status of the participant as healthy

Use of St. John's wort in the 28 days prior to the first study drug administration and during the study

Consumption of any foods or beverages which alter CYP1A2 activity, e.g., barbecued food or cruciferous vegetables, such as broccoli and cauliflower, within 14 days prior to (first) check-in (a list of prohibited foods will be provided to subjects)

Consumption of any foods or beverages containing Seville-type oranges, grapefruit, or poppy seeds within 7 days prior to (first) check-in

Vegetarians, vegans, and/or subjects unable to consume the high-fat breakfast (participants in the food effect evaluation only)

Receipt of blood products within 2 months prior to check-in

Donation of 1 unit of blood to American Red Cross or equivalent organization or donation of over 500 mL of blood in the 56 days prior to the first study drug administration

Donation of plasma in the 7 days prior to the first study drug administration

Poor peripheral venous access

History or significant hypersensitivity to Compound 1 or any related products (including excipients of the formulations) as well as severe hypersensitivity reactions (like angioedema) to any drugs

Subjects who, in the opinion of the Investigator (or designee; including input from subjects' general practitioner, as applicable), should not participate in this study

Subject hospitalized for any reason in a period of 30 days before the start of the study

Subjects who are investigational site staff members or directly involved in the conduct of the study and their family members or subjects who are employed by the Sponsor

### Withdrawal Criteria

### Before First Treatment Administration

Before the first treatment administration, inclusion/exclusion criteria will govern the subjects to be dosed. Subjects withdrawn before first treatment administration will not be followed up and will not undergo End-of-Study/Early Termination assessments. Other safety assessments may be performed if required.

Subjects are free to withdraw their consent to participate in the study at any time, without prejudice. The reason for their withdrawal or for deciding to end their participation will be documented.

### After First Treatment Administration

Subjects may, at any time, voluntarily withdraw from the study or be removed from the study at the discretion of an Investigator or Sponsor. An Investigator may withdraw a subject at any time if it is determined that continuing the study would result in a significant safety risk to the subject or if their behavior is deleterious to the study environment. If such withdrawal occurs, or if the subject fails to return for visits, an Investigator should determine the primary reason for a subject's premature withdrawal from the study and record the reason in the subject's study documents.

A subject will be withdrawn if any of the following criteria are met:

Change in compliance with any inclusion/exclusion criterion that is clinically relevant and affects subject safety as determined by the Investigator (or designee)

Any clinically relevant sign, symptom, or intercurrent illness that, in the opinion of the Investigator (or designee), warrants subject withdrawal

Non-compliance with the study restrictions that might affect subject safety or study assessments/objectives, as considered applicable by the Investigator (or designee)

A subject reports any SAE that is judged as possibly related to the IP by the Investigator (or designee)

A subject experiences increased ALT or AST≥3×ULN with total bilirubin ≥2× ULN, or increased AST ≥3×ULN accompanied by ALP ≥1.5×ULN

A subject has an increase of >60 ms from baseline (predose on Day 1 of each treatment period, as applicable) in QTcF, QTcF >500 ms confirmed by the average of 2 additional ECG recordings performed at least 5 minutes apart, or other clinically significant conduction disturbance or arrhythmia. All ECGs for this determination must be performed under strict resting conditions. Subjects will be required to rest for 10 minutes in supine position prior to ECG collection.

In the event of a subject meeting any of the above withdrawal criteria, the Sponsor's Medical Monitor may deem it necessary for unblinding to occur.

If a subject is withdrawn, the Sponsor will be notified and the date and reason(s) for the withdrawal will be documented in the subject's electronic Case Report Form (eCRF). If a subject is withdrawn, the Investigator should make every effort to perform a final study visit 7 days after the last administration of the investigational product and complete the assessments and procedure outlined in the SOA. Other procedures may be performed at the Investigator's (or designee's) and/or Sponsor's discretion. If the subject is in-house, these procedures should be performed before the subject is discharged from the clinic, where possible. The Investigator (or designee) may also request that the subject return for an additional follow-up visit. All withdrawn subjects will be followed until resolution of all their AEs or until the unresolved AEs are judged by the Investigator (or designee) to have stabilized.

Subjects who are withdrawn for reasons not related to the study drug may be replaced following discussion between the Investigator and the Sponsor. Subjects withdrawn as a result of AEs thought to be related to the study drug will generally not be replaced.

An Investigator may remove a subject from the study on the recommendation of the PK facility and/or sponsor due to an unanticipated event that could result in an inadequately characterized PK profile (e.g., a missed blood draw, an AE, meal deviation, concomitant medication intake, etc.

In the case of a clinically significant illness detected during the trial (including COVID-19 diagnosis), the Principal Investigator (or delegate) will, in concert with the Sponsor, determine the most appropriate course of action on an individual basis. Evaluations will include but are not limited to:

The safety of the subject and other study participants

The possible effect the illness would have on the results gathered during the trial, and their ability to be appropriately analyzed or interpreted

The possibility of suspending participation then re-initiating it after recovery

The implication of any inclusion or exclusion criteria that would contradict possible actions

The implication of any adherence to regulatory guidelines that may be affected by actions decided; for example, group effect analysis

The sample size calculation, current number of subjects, and possibility of replacement subjects

Evaluations and decision-making for subject removal will be documented in the study file, reported to the Sponsor, and discussed where appropriate in the Clinical Study Report.

### Stopping Rules

Participation in the clinical study may be discontinued by an Investigator (or delegate) in charge of the study or by the Sponsor for any of the following reasons in Table 15.

TABLE 15
Stopping Rules

| | | Cohort and/or |
|---|---|---|
| | Individual Subject | Dose Escalation |
| Stopping Rules | Stopping Rules | Stopping Rules |
| ALT or AST ≥3 × ULN | ≥1 subject | N/AP |
| ALT or AST >5 × ULN | N/AP | ≥2 subjects |
| Creatinine >1.2 × ULN or an | ≥1 subject | ≥2 subjects |
| increase in serum creatinine | | |
| by ≥30 μmol/L within 24 | | |
| hours or an increase in serum | | |
| creatinine to ≥1.5 × baseline | | |
| within 7 days | | |
| QTcF >500 msec or change | ≥1 subject | ≥2 subjects |
| from baseline: QTcF >60 | | |
| msec | | |
| Heart rate <45 bpm or heart | ≥1 subject | ≥2 subjects |
| rate >130 bpm^{a} | | |
| Systolic blood pressure <80 | ≥1 subject | ≥2 subjects |
| or systolic blood pressure >155 | | |
| mmHg | | |
| Diastolic blood pressure >100 | ≥1 subject | ≥2 subjects |
| mmHg | | |
| Occurrence of a serious | ≥1 subject | ≥1 subject |
| adverse event considered at | | |
| least possibly related to the | | |
| IP administration | | |
| Possibly drug related severe | N/AP | ≥2 subjects |
| AEs in the same cohort, | | |
| independent of within or not | | |
| within the same system-organ | | |
| class | | |
| Clinically significant drug- | N/AP | ≥2 subjects |
| related laboratory | | |
| abnormalities of the same | | |
| character | | |

^{a}When resting heart rate is between 60-100 beats per minute, use clinical judgment when characterizing bradycardia among some healthy subject populations, for example, conditioned athletes.
Abbreviations: ALT = alanine aminotransferase; AST = aspartate aminotransferase; bpm = beats per minute; msec = millisecond; N/AP = not applicable; ULN = upper limit of normal.

### Trial Stopping Rules

The Investigator must contact the Sponsor immediately to discuss whether to suspend dosing if an AE or laboratory abnormalities indicate that continued dosing of subsequent subjects would not be tolerated or would jeopardize the subjects' safety. The Sponsor alone may suspend dosing at any time for any reason.

### Clinical Trial Stopping Rules:

1. If 1 Compound 1-related SAE occurs

2. If ‘severe’ non-serious adverse reactions (i.e., severe non-serious adverse events considered as, at least, possibly related to Compound 1) in 2 subjects in the same cohort, independent of within or not within the same system-organ-class.

3. One or more subjects fulfill Hy's law defined as increases in aspartate aminotransferase (AST) or alanine aminotransferase (ALT) ≥3× upper limit of normal (ULN) and total bilirubin ≥2×ULN (confirmed with repeat testing) where no other reason can be found to explain the combination of increases, e.g., elevated serum alkaline phosphatase (ALP) indicating cholestasis, viral hepatitis, or administration of another drug.

4. Occurrence of 1 death attributable to the study treatment.

If any of the above scenarios occur, the study will be immediately put on hold. Further discussion will then occur within the SRC, and a safety review will be conducted. Following the SRC review, the study may continue if the Investigator and Sponsor agree it is safe to proceed. If the study is stopped, the MTD will be declared at the dose level lower than that escalation dose.

Lifestyle and/or Dietary Requirements

Subjects will be prohibited from consuming food or beverages containing grapefruit, pomelo, and/or poppy seeds for 7 days prior to the first dosing and during the study.

Subjects will eat only the food provided by the study site during confinement at the CRU.

Subjects will not be permitted to use tobacco or nicotine containing products (including but not limited to; cigarettes, electronic cigarettes, pipes, cigars, chewing tobacco, nicotine patch, or nicotine gum) within 90 days prior to the first study drug administration and must be willing to abstain from nicotine containing products until the follow-up visit.

Subjects will be prohibited from consuming alcohol for 48 hours prior to each dosing and during each study period. Throughout the study, in case of any doubt about alcohol consumption, a test for alcohol may be performed if requested by an Investigator.

Subjects will be prohibited from consuming any foods or beverages which alter CYP1A2 activity (e.g., barbecued food or cruciferous vegetables, such as broccoli and cauliflower) for 7 days prior to check-in (a list of prohibited foods will be provided to subjects).

Subjects must abstain from caffeine for 48 hours prior to check-in and will be prohibited from drinking coffee or caffeine containing beverages throughout the study.

Males who are sexually active will be made aware of the possible male-mediated fetal toxicity associated with the study drug. Male subjects will be expected to use an acceptable contraceptive regimen and not to donate sperm as described in the section Inclusion Criteria.

Subjects will be informed that it is strongly recommended that their female partner of childbearing potential uses one of the following 2 contraceptive methods: Systemic contraceptives (birth control pills, injectable/implantable/insertable hormonal birth control products, transdermal patch) or intrauterine device.

### Concomitant Treatment

In addition to the drugs prohibited as per the Exclusion Criteria section, subjects will also be prohibited from taking any over-the-counter (OTC) products for 14 days prior to the first dosing and for the entire duration of the study.

Medications that are substrates, inhibitors, or inducers of CYP1A2 are specifically prohibited (including, but not limited to, those containing caffeine, tizanidine, theophylline, naproxen, fluvoxamine, cimetidine, fluoroquinolones, or ticlopidine).

Occasional use of paracetamol/acetaminophen at doses ≤2 grams/day will be permitted at the Investigator's discretion.

Except for medication which may be required to treat AEs, no other treatment or medication other than the study drugs will be allowed from the first dosing until all study activities and evaluations have been completed.

Subjects will be instructed to notify the study site about any new medications taken after the start of the study treatment. All medications and significant non-drug therapies (including physical therapy and blood transfusions) administered after the subject has received the study treatment must be listed in the subject case report form (CRF). The drug name and dose taken will be noted. An investigator or delegate and/or the Sponsor will decide whether the subject will be permitted to remain in the study, depending on the drug used, the time of drug intake, etc.

### Study Treatments

Investigational Products: All investigational products, including the placebo, will be provided by the Sponsor. Compound 1 (50 mg oral capsules) will be provided by the Sponsor. Details of study drug preparation, methods of administration, and final volumes will be detailed in study-specific procedures. Placebo: Placebo capsules matched to Compound 1 will be supplied by the Sponsor. The weight is matched to the Sponsor's Compound 1 oral capsules and the composition is the same, including the potentially abuse-deterrent excipients, but without Compound 1.

### Investigational Product Management

Packaging, Labeling and Dispensing: The Sponsor will be responsible for ensuring that the IP is manufactured in accordance with applicable current Good Manufacturing Practice regulations and requirements. The IPs will be labeled according to the requirements of local law and legislation. The IPs will be dispensed by the CRU's pharmacy, unless the Sponsor supplies the pharmacy with prelabeled individual dosing samples.

Storage and Handling: All study drugs will be shipped from the client or client resources to the CRU's pharmacy. Compound 1 should be stored between 2° C. and 8° C. and kept in a tightly closed container. The product should not be used if expired and should not be frozen. The CRU's pharmacy will maintain an inventory record of the IPs received, stored (in a secure restricted area), and dispensed. IPs will be provided to study subjects only.

### Method of Assigning Subjects to Treatment Groups

Randomization codes will be generated with a computer program according to the study design, the number of subjects and the number of treatments. Within each cohort, subjects will be randomized (3:1) to receive Compound 1 or placebo. The random allocation of each IP to each subject will be done in such a way that the study is balanced. Once generated, the randomization code will be final and will not be modified.

Subjects who sign the ICF and are randomized but do not receive the study treatment may be replaced. Subjects, who sign the ICF, are randomized and receive the study treatment, and subsequently withdraw, or are withdrawn or discontinued from the study, will not be replaced.

### Blinding

The randomization code will not be available to the personnel of the bioanalytical facility until the bioanalytical phase of the study has been completed. The treatment assignment will not be known by the study participants.

Furthermore, the randomization code will not be available to the physician and clinical staff involved in the collection, monitoring, revision, or evaluation of AEs, as well as clinical staff who could have an impact on the outcome of the study, and including the pharmacokineticist (or delegate), until all the CRFs have been approved and signed and the bioanalytical phase of the study has been completed.

The preparation of the products will be done by designated personnel that are not directly involved in the clinical aspects of the trial.

The randomization code must not be broken except in emergency situations where the identification of a subject's study treatment is required by an investigator for further treatment to the subject or to complete a SAE report. Randomization information will be held by designated individual(s). The date and reason for breaking the blind must be recorded.

The results of the PK analyses will be made available only to the personnel responsible for evaluating the safety data before proceeding with the next dose level.

### Study Drug Accountability

Complete and accurate inventory records of all study drugs will be maintained. This includes acknowledgment of receipt of each shipment of study product (quantity and condition), subject dispensing records, and returned or destroyed study product.

At the conclusion of the study, all unused investigational products and all medication containers will be returned to the Sponsor unless the Sponsor has approved other arrangements. Drug accountability will be performed at the completion of the trial.

### Administration of Study Drug

The study drug will be administered twice daily (12 hours apart) on Days 1-13 and once in the morning on Day 14. The date and time of each dose will be recorded. For each subject, all scheduled post-dose activities and assessments will be performed relative to the time of the first study drug administration.

An oral dose of the assigned formulation will be administered to subjects with approximately 240 mL of water at ambient temperature. The tablet must be swallowed whole and must not be chewed or broken.

### Treatment Compliance

The study drug will be dispensed only to eligible subjects and administered under the supervision of study personnel. Treatment compliance will be verified according to the site's standard operating procedures (SOPs).

### Meals

Food intake will be controlled for each confinement period and for all subjects.

Subjects will be required to fast overnight for 10 hours prior to the morning dose and for at least 2 hours following dosing. Subjects will also be required to fast for 2 hours prior to the evening dose and 2 hours following dosing. On Day 13, for one cohort (to be determined after the first cohort has completed), subjects will be administered Compound 1 30 minutes after the start of a standardized high-fat, high-calorie meal 30 minutes. Consistent with the meal description outlined in the draft FDA Guidance for Industry: Assessing the Effects of Food on Drugs in INDs and NDAs-Clinical Pharmacology Consideration (2019, accessed 19 May 2022). A high-fat meal is defined as a meal providing approximately 800-1000 calories with approximately 500-600 calories derived from fat. An example high-fat meal consists of 2 eggs fried in butter, 2 strips of bacon, 2 slices of toast with butter, 4 ounces of hash brown potatoes, and 8 ounces of whole milk. Substitutions in this test meal may be made provided that the meal delivers a similar amount of calories from protein, carbohydrate, and fat and has comparable meal volume and texture. Subjects must eat the total content of this meal in 30 minutes or less.

### Fluids

Fluid intake other than water will be controlled for each confinement period and for all subjects.

Each dose of Compound 1 will be administered with approximately 8 oz or 240 mL or water. Water will be permitted as needed except from 1 hour pre-dose until 1 hour after dosing (except for water consumed for the dose administration).

### Other Protocol Restrictions

Subjects will remain in bed (seated or semi-reclined) for at least the first 4 hours following drug administration. However, should AEs occur, subjects may be placed in an appropriate position. During this interval and after the 4-hour period, subjects will be permitted to get up under supervision. Subjects will not engage in strenuous activity at any time during the confinement periods.

### Study Procedures

An overview of the study activities for each participant is detailed in Table 16.

Subjects may leave the clinical site on Day 20. However, they may be advised to stay at the clinical site for safety reasons, if judged necessary by the investigator or delegate in charge.

Unless otherwise stated in the protocol, the Standard Operating Procedures (SOPs) of the study facilities, which are available for all activities relevant to the quality of the study, will be followed during this study. When the nominal time for multiple events occurs simultaneously, the events will be staggered using their acceptable windows (acceptable windows for each assessment are specified in the following sections of this protocol), with priority given to those events related to primary study endpoints.

Any deviation from protocol procedures should be noted in the source documentation and compiled for reporting in the Clinical Study Report.

TABLE 16
Schedule of Activities

| | | | Day | Days | Day | Days | Day | Days | Day | | | Follow- up/EOS |
|---|---|---|---|---|---|---|---|---|---|---|---|---|
| | Screening | Check-in | 1 | 2-3 | 4 | 5-6 | 7 | 8-13 | 14 | Days15-17 | Discharge | visit |
| | Day | | | | | | | | | | | |
| | | | | | | | | | | | | Day 25^{z} |
| Study | Days −28 | | | | | | | | | | Day | (+2 |
| Procedures | to −1 | Day −1 | | | | | | | | | 18 | days) |
| Informed Consent | X | | | | | | | | | | | |
| Inclusion/Exclusion | X | X | | | | | | | | | | |
| Criteria | | | | | | | | | | | | |
| Admission to Unit | | X | | | | | | | | | | |
| Demographic Data | X | | | | | | | | | | | |
| Medical History | X | X^{a} | | | | | | | | | | |
| Psychiatric History | X | | | | | | | | | | | |
| Physical | X | X^{b} | | | | | | | | | X^{p} | |
| Examination | | | | | | | | | | | | |
| Psychiatric | X^{c} | | | | | | | | | | | |
| Examinations^{c} | | | | | | | | | | | | |
| Urinary Drug | X | X | | | | | | | | | | |
| Screen | | | | | | | | | | | | |
| Alcohol Urine Test | X | X | | | | | | | | | | |
| Serology | X | | | | | | | | | | | |
| Pregnancy Test^{d} | X | X | | | | | | | | | X | |
| FSH^{e} | X | | | | | | | | | | | |
| Height and body | X^{f} | | | | | | | | | | X | X |
| weight | | | | | | | | | | | | |
| Genotyping | | | X | | | | | | | | | |
| sample^{g} | | | | | | | | | | | | |
| Exploratory | | | X^{h} | X^{h} | | | X^{h} | | X^{h} | | X | |
| Biomarkers sample | | | | | | | | | | | | |
| Check-in | | X | | | | | | | | | | |
| Check-out | | | | | | | | | | | X | |
| Nonresidential visit | X | | | | | | | | | | | X |
| Randomization | | X | | | | | | | | | | |
| Compound 1 or | | | X | X | X | X | X | X | X | | | |
| Placeboⁱ | | | | | | | | | | | | |
| Plasma PK | | | X | X | X | X | X | X | X | X | 96.00 | X |
| Sampling^{j, k, l} | | | | | | | | | | | | |
| Adverse Event | X | X | X | X | X | X | X | X | X | X | X | X |
| Recording | | | | | | | | | | | | |
| Prior/Concomitant | X | X | X | X | X | X | X | X | X | X | X | X |
| Medication | | | | | | | | | | | | |
| Monitoring | | | | | | | | | | | | |
| Clinical chemistry, | X | X | X | X | X | X | | X | X | X | 96.00 | X |
| Hematology, and | | | | | | | | | | | | |
| Urinalysis^{m} | | | | | | | | | | | | |
| Vital Signs: Blood | X | X | X | X | X | X | X | X | X | X | 96.00 | X |
| Pressure, Pulse | | | | | | | | | | | | |
| Rate and Oral Body | | | | | | | | | | | | |
| Temperature^{n, o} | | | | | | | | | | | | |
| 12-lead ECG^{q, r} | X | | X | X | X | X | X | X | X | X | 96.00 | X |
| VAS Alertness | X | | X | | | | X | | X | | 96.00 | |
| Scale^{s, t} | | | | | | | | | | | | |
| BPRS, CADSS^{t, u, v, x} | X | X | X | | | | X | | X | | | |
| 3MS^{t, v, w, x} | X | X | X | | | | X | | X | | | |
| C-SSRS^{y} | X | X | X | X | | X | X | X | | X | X | X |
| Discharge | | | | | | | | | | | X | |

Abbreviations: BPRS = Brief Psychiatric Rating Scale; CADSS = Clinician-Administered Dissociative States Scale; 3MS = Modified Mini-Mental State; C-SSRS = Columbia-Suicide Severity Rating Scale; ECG = electrocardiogram; FSH = follicle-stimulating hormone; PK = pharmacokinetic(s).
^{a}Interim medical history.
^{b}Symptom directed physical examination and at Investigator discretion.
^{c}Clinical Interview for DSM-5 (SCID-5 CT) will be conducted by appropriately trained staff. This psychiatric examination will include an assessment of premorbid personality, personal and developmental history, alcohol and substance use history, forensic history, past psychiatric history.
^{d}In all females. Serum pregnancy test at Screening and urine pregnancy tests at all other times. A positive urine pregnancy test will be confirmed with a serum pregnancy test.
^{e}In all females to determine postmenopausal status.
^{f}Height measured at Screening only.
^{g}The genotyping samples will be stored and may be processed and analyzed to assess genotyping of drug metabolizing systems.
^{h}Day 1 at pre-dose, 1, 4, and 8 hours; day 2 pre-dose; and days 7 and 14 at pre-dose, 1, 4, and 8 hours.
ⁱDosing twice daily interval from Day 1 to Day 14, two intakes separated by 12.00 hours (note: only the AM dose is administered on Day 14).
^{j}Timing of PK blood samples may be changed based on emerging data.
^{k}At each protocol specified timepoint for PK (including predose baseline) out to 12.00 hours postdose.
^{l}Serial blood will be collected on Day 1, 13 and 14 at predose, 0.5, 1.00, 2.00, 3.00, 4.00, 6.00, 8.00, and 12.00 hours. Trough samples (predose AM) will be collected on Days 2, 5, 7, 9, 11 and 12. Additional PK samples will be drawn after the last dose on Day 14 at 24.00 (Day 15), 36.00 (Day 15), 48.00 (Day 16), 72.00 (Day 17), 96.00 (Day 18) hours postdose and Day 25 at the follow-up EOS visit.
^{m}Day 1 predose, then 24.00 (Day 2) and 72.00 hours (Day 4) postdose, then every 2 days (Days 6, 8, 10, 12, 14, 16).
ⁿDays 1 and 7: Prior to morning dosing and 0.5, 1.00, 2.00, 3.00, 4.00, 6.00, 8.00, and 12.00 hours. Days 2, 4, 6, 8, 10, and 12: Prior to dosing and 4.00 and 12.00 hours postdose. Day 14: Prior to dosing and 0.5, 1.00, 2.00, 3.00, 4.00, 6.00, 8.00, 12.00, 24.00 (Day 15), 48.00 (Day 16), 72.00 (Day 17), 96.00 (Day 18) hours postdose.
^{o}At baseline on Day 1, predose vital sign measurements (except oral body temperature) will be conducted in triplicate (with an interval of approximately 10-30 minutes between each recording). The average value of each parameter will be considered as baseline value.
^{p}Symptom-directed physical examination.
^{q}Days 1, 7, and 14: Predose and at 4.00, 6.00, 8.00, and 12.00 hours postdose. Days 2, 4, 6, 8, 10, and 12: Predose and 4.00 and 12.00 hours postdose. Day 14: Prior to dosing and 0.5, 1.00, 2.00, 3.00, 4.00, 6.00, 8.00, 12.00, 24.00 (Day 15), 48.00 (Day 16), 72.00 (Day 17), 96.00 (Day 18) postdose.
^{r}At baseline in Day 1 predose, ECG recordings will be conducted in triplicate at (−45, −30, and −15 minutes). The average value of each parameter will be considered as baseline value.
^{s}Day 1, 7, and 14: Predose then 4.00, 6.00, and 12.00 hours postdose and Day 18.
^{t}May also be conducted at other times if deemed appropriate based on emerging data.
^{u}Check-in on Day −1, then 6.00 and 24.00 hours postdose on Day 1, Day 7, and Day 14.
^{v}Conducted as part of the psychiatric examination.
^{w}Check-in on Day −1, and 24.00 hours postdose on Day 1, Day 7, and Day 14.
^{x}Subjects will be screened within 28 days prior to dosing. A psychiatric examination will be performed at Screening by a suitably trained psychiatrist or appropriately trained staff using a clinical interview. This will include an assessment of premorbid personality, personal and developmental history, alcohol, and substance use history, forensic history, past psychiatric history, and mental state examination.
^{y}Every other day from Day 1 to discharge.
^{z}Approximately 1 week after last dose.

### Safety Assessments

Safety assessments will include physical examination, psychiatric assessments, vital signs, 12-lead ECG, clinical laboratory tests, and AE monitoring. At the discretion of an investigator, additional safety assessments may be performed as needed to ensure subject safety.

The Investigator or delegate in charge will be present at the clinical site for at least the first 4 hours following first drug administration and will remain available at all times throughout the study.

### Medical History

The medical history at screening will include all queries by the medical and clinical staff related to the subject's well-being and history of relevant past medical events/experiences. Medical history will include all demographic data (age, sex, race, body weight, height, and BMI) and baseline characteristics. Alcohol and smoking habits will also be recorded.

### Physical Examination

A physical examination will be performed by a medically qualified and licensed individual as outlined in Table 16.

The physical examination will include a general review of the following body systems (at minimum): head and neck, cardiovascular, respiratory, gastrointestinal, brief neurological and general appearance, unless a symptom-oriented physical exam is indicated.

### Vital Signs

Vital signs will be measured as outlined in Table 16. Vital signs will be measured within 1 hour prior to dosing. On-study vital signs will be assessed within +/−20 minutes of the nominal time point.

Blood pressure and pulse rate will be measured after being in supine position for at least 3 minutes.

### 12-Lead Electrocardiogram

Twelve-lead ECGs will be performed as outlined in Table 16.

Predose ECGs should be completed 1 hour prior to the morning dose. All other ECG assessments should be completed within +/−20 minutes of the nominal time point.

### Pharmacogenetic and Exploratory Biomarkers Sampling

A blood sample will be collected for CYP 1A2 genotyping on Day 1 as outlined in Table 16. Blood samples will also be collected on Day 1 and during designated times (per Table 16) during the treatment period for exploratory biomarker analysis.

### Laboratory Evaluations

Laboratory evaluations will be performed as outlined in Table 16.

The laboratory evaluations to be conducted for this study are presented in Table 17. Additional clinical laboratory tests may be performed by the medical laboratory as part of larger standard test panels (not required for subject safety).

TABLE 17
Clinical Laboratory Evaluations

| Clinical | |
|---|---|
| Laboratory | |
| Test Panel | Description |
| General | Alanine aminotransferase, albumin, alkaline phosphatase, |
| biochemistry: | aspartate aminotransferase, bilirubin total and conjugated |
| | bilirubin if total bilirubin is elevated, blood urea nitrogen, |
| | calcium, chloride, Creatinine phosphokinase (CPK), Bicarbonate |
| | (CO2), creatinine (including eGFR calculated using the |
| | CKD-EPI calculation, cholesterol total, glucose, LDH, |
| | phosphorous, potassium, protein total, sodium, and uric acid |
| Coagulation: | Prothrombin time (PT)/INR and partial thromboplastin time |
| | (aPTT) levels |
| Endocrinology: | FSH¹ |
| Hematology: | White cell count with differential (absolute values of neutrophil, |
| | lymphocyte, monocyte, eosinophil, and basophil), red cell count, |
| | hemoglobin, hematocrit, mean corpuscular volume, and platelet |
| | count |
| Serology²: | Human immunodeficiency virus (HIV) Ag/Ab Combo, Hepatitis |
| | B surface antigen and Hepatitis C virus |
| Urinalysis: | Color, clarity, specific gravity, pH, leukocyte, protein, glucose, |
| | ketones, bilirubin, blood, nitrite, urobilinogen. Microscopic |
| | examination will only be performed if the dipstick test is outside |
| | of the reference range for leukocyte, blood, nitrite, or protein |
| Urine drug | Alcohol, amphetamines, barbiturates, benzodiazepines, |
| screen: | cannabinoids, cocaine, cotinine, methadone, opiates, |
| | phencyclidine, and tricyclic antidepressants |
| Pregnancy test: | Serum pregnancy test or urine pregnancy test |

¹Screening visit only.

The Investigator or delegate in charge will assess each abnormal value to determine if it is clinically significant. Post-dose clinically significant laboratory values will be reported as AEs, if applicable, as judged by the Investigator or delegate in charge. Only test results required by the protocol and/or abnormal results will be entered in the clinical database and reported in the Clinical Study Report, based on report requirement.

### Brief Psychiatric Rating Scale (BPRS)

The BPRS scale is designed for the assessment of psychiatric symptoms or disorders (e.g., depression, anxiety, hallucinations, and unusual behavior).

The scale must be administered by a psychiatrist or other individual that is suitably qualified by education or training. The assessment will be performed as outlined in Table 16.

The BPRS should be completed prior to Compound 1 administration and at approximately 6.00 and 24.00 hours after dosing on Days 1, 7, and 14.

### Clinician-Administered Dissociative States Scale (CADSS)

The CADSS scale is designed for the assessment of dissociative states in adults.

The scale must be administered by a psychiatrist or other individual that is suitably qualified by education or training. The assessment will be performed as outlined in Table 16.

The CADSS should be completed prior to Compound 1 administration and at approximately 6.00 and 24.00 hours after dosing on Days 1, 7, and 14.

### Columbia Suicide Severity Rating Scale (C-SSRS)

The C-SSRS is a questionnaire designed for the assessment of suicidal ideation and behavior in adolescents and adults.

To monitor for a history of (the past 2 years to present) or for the emergence of suicidal ideation and behavior, subjects will undergo C-SSRS evaluations at the time points indicated in Table 16.

The questionnaire must be administered by a psychiatrist or other individual that is suitably qualified by education or training.

The C-SSRS should be completed prior to Compound 1 administration and will be assessed on Days 1, 3, 5, 7, 9, 11, 13, 15, 17, on day 18 prior to discharge, and at the follow-up/EOS visit.

If there is a positive result for suicidality on the C-SSRS after Screening (defined by a subject answering “yes’ to questions 4 or 5 on the suicidal ideation portion of the C-SSRS), the subject will be evaluated by an investigator or medically qualified sub-investigator for continuation in the study.

If a subject becomes suicidal during the study, an investigator or medically qualified sub-investigator should provide the appropriate treatment to the subject.

### Modified Mini-Mental State Examination (3 MS)

The 3 MS is an examination designed for the assessment of dementia in adults.

The examination must be administered by a psychiatrist or other individual that is suitably qualified by education or training. The assessment will be performed as outlined in Table 16.

The 3 MS should be completed prior to Compound 1 administration and at approximately 24.00 hours after dosing on Days 1, 7, and 14.

### Visual Analogue Alertness Scale (VAS)

The VAS alertness scale is designed to assess the alertness of subjects across a continuum scale with vales from 0 to 10.

The scale must be administered by a psychiatrist or other individual that is suitably qualified by education or training. The assessment will be performed as outlined in Table 16.

The VAS should be completed prior to Compound 1 administration and at approximately 4.00, 6.00, and 12.00 hours after dosing on Days 1, 7, and 14. This assessment will also occur on Day 18 prior to discharge.

### Rescue Therapy

A crash cart with the necessary resuscitation equipment, including parenteral naloxone, will be available in case of a medical emergency.

### Pharmacokinetic Assessments

A total of 37 blood samples will be collected for PK assessments. The complete blood sampling schedule is presented in Table 18.

TABLE 18
Pharmacokinetic Blood Sampling Schedule

| Day | Nominal Time^{a} (hours) |
|---|---|
| Days 1 and 13^{a} | 0.00 |
| | 0.50 |
| | 1.00 |
| | 2.00 |
| | 3.00 |
| | 4.00 |
| | 6.00 |
| | 8.00 |
| | 12.00 |
| Days 2, 5, 7, | 0.00 |
| 9, 11, and 12 | |
| Day 14 | 0.00 |
| | 0.50 |
| | 1.00 |
| | 2.00 |
| | 3.00 |
| | 4.00 |
| | 6.00 |
| | 8.00 |
| | 12.00 |
| Day 15 | 24.00 |
| | 36.00 |
| Day 17 | 72.00 |
| Day 18 | 96.00 |
| Day 25 + 2 days | 264.00 |

^{a}Nominal times listed are relative to the morning dose.

Blood samples will be collected by direct venipuncture into a labeled tube containing the appropriate anticoagulant as specified by the bioanalytical facility. As an option to the subject or if judged necessary by the clinical staff, blood samples may be collected from an indwelling cannula, which will be placed in the vein of the subject.

The time of PK blood sample collection will be calculated relative to the time of treatment administration. The actual time of all PK blood draws will be recorded and reported for all subjects.

Windows for timed PK blood sample collections are presented in Table 19. PK samples collected outside of the pre-specified windows will be documented as protocol deviations. Since actual times are to be used for the PK analysis, deviations will be reflected in the analysis unless indicated otherwise upon review of the data.

TABLE 19
Acceptable Windows for Timed PK Blood
Specimen Collection Procedures

| Elapsed Time | Accepted Window | |
|---|---|---|
| Pre-dose | No window specified | |
| >0 hour to ≤30 minutes | ±1 | minute |
| >30 minutes to ≤4 hours | ±2 | minutes |
| >4 hours to ≤12 hours | ±5 | minutes |
| >12 hours to ≤24 hours | ±10 | minutes |
| >24 hours to ≤72 hours | ±2 | hours |
| >72 hours | ±4 | hours |

Compound 1 concentrations for PK assessments will be obtained through bioanalysis of the plasma derived from the blood samples drawn during this study, using a validated bioanalytical method.

### Pharmacokinetic Sample Processing, Storage and Shipping

Blood samples for PK determination will be processed, stored, and shipped according to the sample processing instructions supplied by the bioanalytical facility.

### Adverse Events Documentation

### Definitions

An AE is defined as any untoward medical occurrence in a subject administered an investigational product and which does not necessarily have a causal relationship with the treatment. An AE can therefore be any unfavorable and unintended sign (including a clinically significant abnormal clinical laboratory finding, for example), symptom, or disease temporally associated with the use of an investigational product, whether or not related to the investigational product.

A suspected adverse reaction (SAR) is any AE for which there is a reasonable possibility the drug caused the AE. ‘Reasonable possibility’ means there is evidence to suggest a causal relationship between the drug and the AE. A suspected adverse reaction implies a lesser degree of certainty about causality than adverse reaction, which means any AE caused by a drug.

An AE may be:
A new illness;
Worsening of a concomitant illness;
An effect of the study drug including comparator; it could be an abnormal clinical
laboratory value as well as a significant shift from baseline within normal range which an investigator considers to be clinically important;
Surgical procedures themselves are not AEs. They are therapeutic measures for conditions that required surgery. The condition for which the surgery is required is an AE, if it occurs or is detected during the study period. Planned surgical measures permitted by the clinical study protocol and the conditions(s) leading to these measures are not AEs, if the condition(s) was (were) known before the start of study treatment. In the latter case, the condition should be reported as medical history.

A SAE or reaction is any untoward medical occurrence that at any dose:
Results in death;
Is life-threatening;
Requires inpatient hospitalization or prolongation of existing hospitalization;
Results in persistent or significant disability or incapacity (defined as a substantial disruption of a person's ability to conduct normal life functions);
Is a congenital anomaly or birth defect;
Is an important medical event that may jeopardize the subject or may require intervention to prevent one of the other outcomes listed above (according to medical judgment of an investigator).

### Severity Assessment

All AEs will be graded per the FDA's Guidance for Industry on Toxicity Grading for Healthy Adult and Adolescent Volunteers Enrolled in Preventive Vaccine Clinical Trials.

Every effort will be made to obtain an adequate evaluation of the severity.

### Causality Assessment

An Investigator will determine the relationship of any AE to the study drug using the guidelines presented in Table 20.

TABLE 20
Adverse Event Relationship to Study Drug

| Relationship | |
|---|---|
| to Drug | Comment |
| Reasonable | A temporal relationship exists between the AE |
| Possibility | onset and administration of the investigational product |
| | that cannot be readily explained by the subject's |
| | clinical state orconcomitant therapies. |
| | Furthermore, the AE appears with some degree |
| | of certainty to be related, based on the known |
| | therapeutic and pharmacologic actions or AE |
| | profile of the investigational product. |
| | In case of cessation or reduction of the dose the |
| | AE may abate or resolve and it may reappear upon |
| | rechallenge. |
| No Reasonable | Evidence exists that the AE has an etiology other |
| Possibility | than the investigational product. |
| | For SAEs, an alternative causality must be provided |
| | (e.g., preexisting condition, underlying disease, |
| | intercurrent illness, or concomitant medication). |

AE = adverse event;
IP = investigational product;
SAE = serious adverse event

### Adverse Event Monitoring

For the purposes of this study, the monitoring period for AEs extends from Screening until the EOS visit. From screening to the first dose of the study, AEs will be recorded as screening events or as part of the medical history, as applicable. AEs occurring after initiation of study drug will be indicated as TEAEs in the clinical study report.

Subjects will be questioned on their health status at the beginning of each study period and before each departure from the clinical site. Open-ended questions will be asked.

During the study, all AEs spontaneously reported by the subject, observed by the clinical staff, or elicited by general questioning will be recorded for all subjects and reported in the CRF.

If necessary, every effort will be made to obtain an adequate follow-up of the subjects. Should any subject choose to withdraw from the study, they will be advised of the safety precautions to be taken.

Any AE which remains unresolved as of the last visit will require an evaluation and follow-up until the AE has been resolved or a reasonable explanation for its persistence found or is deemed mild and safely resolving.

In the case of AEs deemed related to the Investigational Product, every effort will be made to determine the final outcome.

It is an investigator's responsibility to ensure subjects experiencing AEs receive appropriate follow-up, treatment where required, and that every action is well documented.

Classification of AEs will be performed by System Organ Class (SOC) and Preferred Term (PT) using the Medical Dictionary for Regulatory Activities (MedDRA), version 25.0 or higher.

Concomitant medications will be coded using the World Health Organization drug dictionary (WHO-DDE March 2021 or later).

### Reporting of Pregnancy

Pregnancy in a female study subject shall be reported to the Sponsor within 24 hours of the knowledge of its occurrence by an investigator or delegate (for pregnancies occurring during the course of the study or immediately following the end of the study). Because of the possibility the fetus/embryo could have been exposed to the study drug through the parent and for the subject's safety, the pregnancy will be followed up to determine its outcome, including spontaneous or voluntary termination, details of birth, presence or absence of any birth defects, congenital anomalies, or maternal and/or newborn complications.

Pregnancy that occurs within 90 days after the follow-up visit in a female partner of a male study subject shall be reported to the Sponsor within 24 hours of the knowledge of its occurrence by the clinical site that such pregnancy occurred during the course of the study or right after. Because of the possibility that the fetus/embryo could have been exposed to the study drug through the parent and for the safety of the subject's female partner, the pregnancy will be followed up to determine its outcome, including spontaneous or voluntary termination, details of birth, presence or absence of any birth defects, congenital anomalies, or maternal and/or newborn complications.

The pregnancy will be recorded and reported by the clinical site to the Sponsor. Pregnancy follow-up will also be properly recorded to ensure quality and completeness of the data belonging to the study drug and will include an assessment of the possible causal relation between the study drug and any pregnancy outcome. Any SAE experienced during pregnancy will be reported on an SAE Report Form.

### Serious Adverse Event Reporting

The CRU will notify any SAE to the Sponsor, without regard to causality, within 24 hours after becoming aware of its occurrence.

If, during follow-up, any non-serious AE worsens and eventually meets the criteria for an SAE, that AE should be recorded as a new SAE.

The initial SAE report must be as complete as possible, including details of the current illness and SAE, and an assessment of the causal relationship between the event and the investigational product(s). Information not available at the time of the initial report (e.g., an end date for the AE, laboratory values received after the report, or hospital discharge summary) must be documented. All follow-up information must be reported as soon as the relevant info is available.

An SAE will be considered “unexpected” if the AE is not listed in the investigator brochure or is not listed at the specificity or severity that has been observed; or, if an investigator brochure is not required or available, is not consistent with the risk information described in the general investigational plan or elsewhere in the current application. “Unexpected,” as used in this definition, also refers to AEs that are mentioned in the investigator brochure as occurring with a class of drugs or as anticipated from the pharmacological properties of the drug but are not specifically mentioned as occurring with the particular drug under investigation.

The CRU will determine whether any serious unexpected, related AE must be reported to the Institutional Review Board (IRB). If so, the event will be reported via fax or email within 15 calendar days of an investigator or staff becoming aware of the event.

The Sponsor will determine whether the SAE must be reported in an expedited manner to the applicable regulatory agencies. If so, the Sponsor will report the event to those agencies and to all participating investigators.

If reports of any new and unexpected AEs become available to the Sponsor during the clinical portion of this study (related or not to the present study), the Sponsor has to advise the CRU, through its clinical investigator, of those events.

### Data Analysis and Statistical Considerations

### Analysis Populations

### Safety Population

The safety population will include all subjects who received at least 1 dose of one of the investigational product or placebo.

The number of subjects who were included, who discontinued, and who completed the study will be tabulated. The primary reasons for discontinuation will be provided.

### Pharmacokinetic Population

In most cases, the decision of which subjects will be included in the PK analysis is to be taken before the start of the sample analysis by the bioanalytical facility.

The PK population will be described in an SAP. Generally, the PK population includes all the subjects who received the study drug or equivalent and have at least one post-dose evaluable concentration values in any biological matrix.

### Demographic Data and Other Baseline Characteristics

Statistics for demographic and baseline data will be detailed in an SAP.

### Safety

### Safety Endpoints

The safety endpoint will be assessed by the number, severity, and type of TEAEs.

### Safety Analysis

The clinical laboratory tests and the measurements of vital signs, ECGs, physical examinations, psychiatric assessments, and other safety parameters will be used to perform the safety statistical analysis.

### Safety Statistical Methodology

Statistics for summary of AEs and safety results will be fully detailed in an SAP.

### Pharmacokinetics

The PK analysis will be carried out according to SOPs. Pharmacokinetic data handling and statistical analysis will be fully detailed in an SAP.

### Pharmacogenetic Statistical Methodology

The exploratory pharmacogenetic studies are designed to investigate the association between genetic factors (genotypes) and clinical assessments (phenotypes) which are collected during the clinical trial. Without prior evidence of a strong association, a number of possible associations are evaluated with exploratory analyses. A range of statistical tests (chi-square tests, ANCOVAs, linear and logistic regression) are used for the analyses. Additional data from subsequent clinical trials are often needed to confirm associations. Alternatively, if the numbers of subjects enrolled in the study are too small to complete proper statistical analyses, these data may be combined, as appropriate, with those from other studies to enlarge the data set for analysis.

### Planned Interim Analyses

No formal interim analyses will be performed; blinded safety data will be reviewed by an Investigator and the Sponsor's Medical Monitor following completion of each Compound 1 dose level.

The SAP will describe the planned interim analyses in greater detail.

### Determination of Sample Size

No formal sample size analysis was performed. It is estimated that approximately 24 subjects should be sufficient to meet the objectives of the study.

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

### Example 6—Amendment to Phase 1, Randomized, Placebo Controlled, Multiple Ascending Dose (MAD) Study to Evaluate the Safety, Tolerability, and Pharmacokinetics of Compound 1 in Healthy Subjects

Example 6 presents another randomized, placebo controlled, multiple ascending dose (MAD) study protocol. Example 6 is the same as Example 5 except that the study will evaluate Compound 1 at 50 mg once a day for 14 consecutive days in Cohort 2, with the potential to increase the dose in subsequent cohorts based on the emerging safety and pharmacokinetic data. The study will also include an exploratory objective to evaluate the role that inhibition of the Cytochrome P1A2 (CYP1A2) enzyme plays in determining steady state exposure of Compound 1.

### Example 7—A Phase 2, Randomized, Global, Double-Blind, Placebo-Controlled, Parallel-Group Study to Evaluate Whether Compound 1 Reduces Cocaine Use in Patients Diagnosed with Cocaine Use Disorder

This is a randomized, subject and investigator-blinded, parallel-group, placebo-controlled study to assess the safety, tolerability, pharmacokinetics (PK), and efficacy of Compound 1 in subjects with at least moderate CUD who use cocaine through snorting (intranasally) as the primary route of administration.

### Study Design/Methodology

The study will consist of a 14-day screening period, followed by a 14-day baseline period, a 12-week outpatient treatment period, and an end of study visit approximately 14 days after the last study drug administration. The total duration for each subject in the study will be approximately 18 weeks including screening and baseline. The entire study is to be run in an outpatient setting.

Screening Period: After signing an informed consent form, potential enrollees will undergo a medical history, physical exam, laboratory, and other assessments to determine their eligibility for study participation. Individuals who meet all the inclusion criteria, and none of the exclusion criteria will be enrolled in the study.

Baseline Period: Prior to randomization, all enrolled subjects will undergo baseline (pre-treatment) assessments including safety labs, quantification of substance usage, and health questionnaires. Upon confirmation of study eligibility, individuals will enter the treatment period.

Treatment Period: Enrolled individuals will be randomized to receive either Compound 1 or matching placebo daily for 12 weeks. Subjects will record their daily cocaine consumption throughout the entire treatment period. At regular intervals, participants will return to the clinical study site for safety, PK, PD, and efficacy assessments.

End of Study: Approximately 2 weeks after treatment completion, subjects will return to the clinical study site for an end of study visit. At this time each individual will undergo follow up assessments including physical examination, safety labs, and cocaine use and other health questionnaires.

### Objectives and Endpoints

Primary: To evaluate treatment effect of 12-week Compound 1 administration in reducing cocaine use. Endpoint: Proportion of cocaine use days by Timeline Follow-Back (TLFB) cocaine self-report.

Secondary Objective 1: (1) To assess the effect of Compound 1 on: (a) other measures of cocaine use; (b) measures of alcohol use. Endpoints: (1) Proportion of Positive Urine Measurements of Benzoylecgonine (BE); (2) Change from baseline in Cocaine and other substances cravings by the BSCS (Brief substance craving scale); (3) Change from baseline in the proportion of heavy drinking days per week as assessed by the TLFB; (4) Change from baseline in the proportion of heavy drinking days in a month (where a heavy drinking day is defined as any day in which a patient consumed ≥5 standard alcohol drinks for men and ≥4 standard alcohol drinks for women).

Secondary Objective 2: To assess the safety and tolerability of Compound 1. Endpoint: Vital signs, ECG parameters, clinical safety laboratory parameters (chemistry/hematology/urinalysis), (serious) AEs reporting, suicidal ideations (CSSR-S), BPRS.

Secondary Objective 3: To evaluate the pharmacokinetics of Compound 1 in subjects with CUD. Endpoint: Plasma concentrations of Compound 1.

Exploratory Objective 1: To assess the frequency of other drugs and alcohol and nicotine use before and during 12-week Compound 1 treatment. Endpoints: (1) Hair drug test for cocaine, amphetamine, methamphetamine, MDMA, MDEA, MDA, methylphenidate, ketamine, and EtG; (2) Change from Baseline to Month 3 in alcohol biomarkers of consumption and/or relapse (LFTs, % CDT and urine EtG); (3) Fagerström Test for Nicotine Dependence and urine cotinine.

Exploratory Objective 2: To examine whether individual genetic variation in genes relating to drug metabolism and transporters, CUD, and the drug target pathway confirm differential response to Compound 1. Endpoint: Single genetic variant or genetic risk score and associations with outcome measures (pharmacogenetics blood samples).

Exploratory Objective 3: To assess the effect of 12-weeks Compound 1 administration versus placebo on: (a) Depressive symptoms, (b) Anxiety symptoms, (c) Stress, (d) Sleep, (e) Global Functioning. Endpoints: (1) Patient Reports Outcomes (PRO) and wearable device data: (a) Beck's Depression Inventory (BDI-II); (b) State-Trait Anxiety Inventory (STAI) and digital assessment of heart rate variability; (c) Digital assessment of electrodermal activity; (d) Insomnia Severity Index (ISI) and digital assessment of total sleep duration and overall sleep quality; (2) Clinician Reported Outcome: (e) CGI-S and CGI-I.

### Population

Key Inclusion Criteria: Patients who meet the following inclusion criteria will be eligible to participate in the study:

(1) Adult male or female subjects 18 to 65 years of age, inclusive.

(2) Must use cocaine through snorting (intranasally) as the primary route of administration.

(3) Has met the DSM-5 diagnosis (using the SCID-5-CT diagnostic interview) of at least a moderate CUD (4 or more criteria) for the 3-months immediately prior to signing the informed consent form.

(4) Recent cocaine use confirmed by a positive urine screen for 1 or more benzoylecgonine (BE).

(5) Must be seeking treatment for cocaine dependence and have a desire to reduce or cease cocaine use as per goals assessed at baseline.

(6) Must be abstinent from cocaine use for at least 3 days preceding first dosing (Day 1) as assessed by self-report TLFB. The 2 urine drug screen samples at visit 2 (second screening) and visit 3 (baseline) must show a decrease in BE levels or must be both negative.

Key Exclusion Criteria: Patients who meet any of the following exclusion criteria will be excluded from participation in the study:

Score “yes” on item 4 or item 5 of the Suicidal Ideation section of the C-SSRS, if this ideation occurred in the past 6 months, or “yes” on any item of the Suicidal Behavior section, except for the “Non-Suicidal Self-Injurious Behavior” (item also included in the Suicidal Behavior section) if this behavior occurred in the past 2 years.

(1) Score “yes” on item 4 or item 5 of the Suicidal Ideation section of the C-SSRS, if this ideation occurred in the past 6 months, or “yes” on any item of the Suicidal Behavior section, except for the “Non-Suicidal Self-Injurious Behavior” (item also included in the Suicidal Behavior section) if this behavior occurred in the past 2 years.

(2) Has current diagnosis of moderate or severe substance use disorder (according to DSM-5) on alcohol, cannabis, opioids, or other stimulants, except cocaine. Note: Nicotine use is allowed. Current use of alcohol and/or cannabis is allowed. Current use of amphetamine and/or opiates is excluded, however, lifetime use is allowed).

(3) Meets current or lifetime DSM-5 criteria for schizophrenia or any psychotic disorder or organic mental disorder. Note: Subjects diagnosed with other psychiatric disorders maybe included at the discretion of the PI provided that the concurrent treatment for the comorbid psychiatric condition will not interfere with completion of the study or place the patient at heightened risk through participation in the trial.

(4) Currently receiving treatment for substance use disorder (e.g., disulfiram, acamprosate, methylphenidate, modafinil, topiramate, immediate-release dexamphetamine, or baclofen) in the last 90 days.

(5) Requires treatment with any psychoactive medications, including any anti-seizure medications (with the exception of medications used for short-term treatment of insomnia) Note: SSRIs and benzodiazepines are allowed if they have adequate stable dose for at least 1 month prior to study treatment dosing

### Dosage Forms and Route of Administration

Score Subjects will be assigned to one of the following 2 treatments arms in a 1:1 ratio. (1) Test product: Compound 1 mg capsules (Dose to be determined) (2) Reference product: placebo capsules.

Administration route: Compound 1 or placebo will be administered orally.

### Evaluation Criteria

Efficacy (Pharmacodynamics): (1) Proportion of cocaine use days delivered from TLFB cocaine self-report; (2) Alcohol consumption as delivered from TLFB; (3) Alcohol self-report (HDD per week and per month); (4) Measurement of cocaine's main metabolite benzoylecgonine (BE) in urine.

Pharmacokinetics: (5) Compound 1 plasma concentrations pre- and post-morning dose.

Safety: (6) Physical examinations including vital signs, height, and weight; (7) Laboratory evaluations (chemistry/hematology/urinalysis); (8) Electrocardiograms (ECGs); (9) Suicidal ideation, and behavior as assessed by the Columbia-Suicide Severity Scale (C-SSRS); (10) Brief Psychotic Rating Scale (BPRS); (11) Serious adverse events (SAEs) and adverse events (AEs) reporting.

Other assessments: (12) Hair drug test for cocaine, amphetamine, methamphetamine, MDMA, MDEA, MDA, methylphenidate, ketamine) and Ethyl glucuronide (EtG); (13) Alcohol biomarkers (LFTs, % CDT, urine EtG); (14) Pharmacogenetics blood samples;

Patient Reported Outcome (PRO): (15) Beck's Depression Inventory (BDI-II); (16) State-Trait Anxiety Inventory (STAI); (17) Insomnia Severity Index (ISI); (18) Brief Substance craving Scale (BSCS).

Clinician Reported Outcome (CRO): (17) Clinical Global Impression Scale-Severity (CGI-S) and Improvement (CGI-I); (18) Digital assessment via wearables/smartphone (Using passively collected data from smart device/sensor); (19) Total sleep duration and overall sleep quality; (20) Heart rate variability (HRV); (21) Electrodermal activity (EDA).

### Sample Size Determination

Approximately 90 subjects will be enrolled to ensure 70 subjects (35 subjects on Compound 1 and 35 subjects on placebo) complete the study. A 90% power at a Type I error of 5% is assumed with a standard deviation of 25% and a 30% dropout rate.

### Example 8—A Phase 2, Randomized, Double-Blind, Placebo-Controlled, Parallel-Group Study to Evaluate Whether Compound 1 Reduces Opioid Use in Patients Diagnosed with Opioid Use Disorder

This is a randomized, subject and investigator-blinded, parallel-group, placebo-controlled study to assess the safety, tolerability, pharmacokinetics (PK), and efficacy of Compound 1 in subjects with at least moderate OUD. Eligible patients must currently be on buprenorphine treatment for OUD and express a desire to be opioid abstinent and are willing to undergo a buprenorphine taper and able to discontinue buprenorphine, where relevant.

### Study Design/Methodology

The study will consist of up to a 3-day screening period (which will occur in parallel to buprenorphine taper), followed by a 12-week outpatient treatment period, and an EOS evaluation visit approximately 14 days after the last study drug administration. The total duration for each subject in the study will be approximately 14.5 weeks including screening. The entire study is to be run in an outpatient setting.

Screening/Baseline Period: After signing an informed consent form, potential enrollees will undergo a medical history, physical exam, laboratory, and other assessments to determine their eligibility for study participation. Individuals who meet all the inclusion criteria, and none of the exclusion criteria will be enrolled in the study. During this time, relevant patients will undergo a buprenorphine taper/discontinuation.

Treatment Period: Enrolled individuals will be randomized to receive either Compound 1 or matching placebo daily for 12 weeks. Subjects will record their daily opioid consumption throughout the entire treatment period. At regular intervals, participants will return to the clinical study site for safety, PK, PD, and efficacy assessments.

End of Study: Approximately 2 weeks after treatment completion, subjects will return to the clinical study site for an end of study visit. At this time each individual will undergo follow up assessments including physical examination, safety labs, and opioid use and other health questionnaires.

### Objectives and Endpoints

Primary Objective: To evaluate treatment effect of 12-week Compound 1 administration in reducing opioid use. Endpoint: Proportion of opioid use days by Timeline Follow-Back (TLFB) opioid self-report.

Secondary Objective 1: To assess the effect of 12 weeks Compound 1 on: (a) Other measures of opioid use; (b) Measures of alcohol use. Endpoints: (1) Measurement of opioid metabolites in urine and hair; (2) Change from baseline in Opioid and other substances cravings by the BSCS (Brief substance craving scale); (3) TLFB alcohol self-report; urinalysis (Ethyl Glucuronide (EtG)); (4) Change from baseline in the proportion of heavy drinking days per week as assessed by the TLFB; (5) Change from baseline in the proportion of heavy drinking days in a month (where a heavy drinking day is defined as any day in which a patient consumed ≥5 standard alcohol drinks for men and ≥4 standard alcohol drinks for women). This data will be calculated from the TLFB data with Month 3 being the time point of interest.

Secondary Objective 2: To assess the safety and tolerability of Compound 1. Endpoints: Vital signs, ECG parameters, clinical safety laboratory parameters (chemistry/hematology/urinalysis), (serious) AEs reporting, suicidal ideations (CSSR-S), BPRS.

Secondary Objective 3: To evaluate the PK of Compound 1 in subjects with OUD. Endpoints: Plasma concentrations of Compound 1.

Exploratory Objective 1: To assess the frequency of other drugs and alcohol and nicotine use before and during 12-week Compound 1 treatment. Endpoints: (1) Hair drug test for amphetamine, methamphetamine, MDMA, MDEA, MDA, methylphenidate, ketamine, and EtG; (2) Change from Baseline to Month 3 in alcohol biomarkers of consumption and/or relapse (LFTs, % CDT and urine EtG); (3) Fagerström Test for Nicotine Dependence and urine cotinine

Exploratory Objective 1: To examine whether individual genetic variation in genes relating to drug metabolism and transporters, OUD, and the drug target pathway confirm differential response to Compound 1 Endpoints: Single genetic variant or genetic risk score and associations with outcome measures (pharmacogenetics blood samples)

Exploratory Objective 1: To assess the effect of 12-weeks Compound 1 on: (a) Depressive symptoms; (b) Anxiety symptoms; (c) Stress; (d) Sleep; (e) Global Functioning. Endpoints: (1) Patient Reports Outcomes (PRO) and wearable device data: (a) Beck's Depression Inventory (BDI-II); (b) State-Trait Anxiety Inventory (STAI) and digital assessment of heart rate variability; (c) Digital assessment of electrodermal activity; (d) Insomnia Severity Index (ISI) and digital assessment of total sleep duration and overall sleep quality; (2) Clinician Reported Outcome: (e) CGI-S and CGI-I.

### Population

Key Inclusion Criteria; Patients who meet the following inclusion criteria will be eligible to participate in the study:

(1) Adult male or female subjects 18 to 65 years of age, inclusive.

(2) Have an opiate use disorder that meets criteria set in the Mini-International Neuropsychiatric Interview (MINI 7.0.2) for DSM-5≥three months before screening.

(3) Current inpatient opioid discontinuation program with the use of buprenorphine taper which the subject is willing and able to discontinue prior to randomization into the study.

(4) Must have a desire to reduce or cease opioid use and express a desire to be opioid abstinent without the use of current approved treatments for opioid use disorder.

(5) Self-report of opioid use in the 30 days before screening confirmed by a positive urine screen (verified by site records).

Key Exclusion Criteria: Patients who meet any of the following exclusion criteria will be excluded from participation in the study:

(1) Score “yes” on item 4 or item 5 of the Suicidal Ideation section of the C-SSRS, if this ideation occurred in the past 6 months, or “yes” on any item of the Suicidal Behavior section, except for the “Non-Suicidal Self-Injurious Behavior” (item also included in the Suicidal Behavior section) if this behavior occurred in the past 2 years.

(1) Has current diagnosis of moderate or severe substance use disorder (according to DSM-5) other than OUD or Tobacco Use Disorder in the 3 months prior to screening.

(2) Positive urine drug screen for cocaine and/or methamphetamines.

(3) Current diagnosis other than opioid use disorder requiring chronic opioid treatment.

(4) Current medication-assisted treatment with methadone or naltrexone.

(5) Received extended-release buprenorphine (Sublocade) within 300 days of enrollment.

(6) Acute opioid withdrawal symptoms, as defined by a score on the COWS >4.

(7) Meets current or lifetime DSM-5 criteria for schizophrenia or any psychotic disorder or organic mental disorder. Note: Subjects diagnosed with other psychiatric disorders maybe included at the discretion of the PI provided that the concurrent treatment for the comorbid psychiatric condition will not interfere with completion of the study or place the patient at heightened risk through participation in the trial.

(8) Currently receiving treatment for substance use disorder other than buprenorphine (e.g., disulfiram, acamprosate, methylphenidate, modafinil, topiramate, immediate-release dexamphetamine, or baclofen) in the last 90 days.

(9) Has had >5 previous relapses.

(10) Requires treatment with any psychoactive medications, including any anti-seizure medications (with the exception of medications used for short-term treatment of insomnia.) Note: SSRIs and benzodiazepines are allowed if they have adequate stable dose for at least 1 month prior to study treatment dosing.

### Dosage Forms and Route of Administration

Subjects will be assigned to one of the following 2 treatments arms in a 1:1 ratio: (1) Test product: Compound 1 capsules (Dose to be determined); (2) Reference product: placebo capsules

Administration route: Compound 1 or placebo will be administered orally.

### Evaluation Criteria

Efficacy (Pharmacodynamics): (1) Proportion of opioid use days delivered from TLFB opioid self-report; (2) Alcohol consumption as delivered from TLFB (paper and electronic); (3) Alcohol self-report (HDD per week and per month); (4) Measurement of opioid metabolites in urine.

Pharmacokinetics: (5) Compound 1 plasma concentrations pre- and post-morning dose.

Safety: (6) Physical examinations including vital signs, height, and weight; (7) Laboratory evaluations (chemistry/hematology/urinalysis); (8) Electrocardiograms (ECGs); (9) Suicidal ideation, and behavior as assessed by the Columbia-Suicide Severity Scale (C-SSRS); (10) Serious adverse events (SAEs) and adverse events (AEs) reporting.

Other assessments: (11) Hair drug test for opioid, amphetamine, methamphetamine, MDMA, MDEA, MDA, methylphenidate, ketamine) and Ethyl glucuronide (EtG); (12) Alcohol biomarkers (LFTs, % CDT, urine EtG); (13) Pharmacogenetics blood samples.

Patient Reported Outcome (PRO): (14) Beck's Depression Inventory (BDI-II); (15) State-Trait Anxiety Inventory (STAI); (16) Insomnia Severity Index (ISI); (17) Brief Substance craving Scale (BSCS).

Clinician Reported Outcome (CRO): (18) Clinical Global Impression Scale-Severity (CGI-S) and Improvement (CGI-I).

Digital assessment via wearables/smartphone (Using passively collected data from smart device/sensor): (19) Total sleep duration and overall sleep quality; (20) Heart rate variability (HRV); (21) Electrodermal activity (EDA).

### Sample Size Determination

Approximately 90 subjects will be enrolled to ensure 70 subjects (35 subjects on Compound 1 and 35 subjects on placebo) complete the study. A 90% power at a Type I error of 5% is assumed with a standard deviation of 25% and a 30% dropout rate.

### Example 9—A Phase 2, Randomized, Double-Blind, Placebo-Controlled, Parallel-Group Study to Evaluate Whether Compound 1 Reduces Alcohol Use in Patients Diagnosed with Alcohol Use Disorder

This is a randomized, subject and investigator-blinded, parallel-group, placebo-controlled study to assess the safety, tolerability, pharmacokinetics (PK), and efficacy of Compound 1 in subjects with at least moderate alcohol use disorder (AUD).

### Study Design/Methodology

The study will consist of a 14-day screening period, followed by a 7-day baseline period, a 12-week outpatient treatment period, and an end of study visit approximately 14 days after the last study drug administration. The total duration for each subject in the study will be approximately 16 weeks including screening and baseline. The entire study is to be run in an outpatient setting.

Screening Period: After signing an informed consent form, potential enrollees will undergo a medical history, physical exam, laboratory, and other assessments to determine their eligibility for study participation. Individuals who meet all the inclusion criteria, and none of the exclusion criteria will be enrolled in the study.

Baseline Period: Prior to randomization, all enrolled subjects will undergo baseline (pre-treatment) assessments including safety labs, quantification of substance usage, and health questionnaires. Upon confirmation of study eligibility, individuals will enter the treatment period.

Treatment Period: Enrolled individuals will be randomized to receive either Compound 1 or matching placebo daily for 12 weeks. Subjects will record their daily alcohol consumption throughout the entire treatment period. At regular intervals, participants will return to the clinical study site for safety, PK, PD, and efficacy assessments.

End of Study: Approximately 2 weeks after treatment completion, subjects will return to the clinical study site for an end of study visit. At this time each individual will undergo follow up assessments including physical examination, safety labs, and alcohol use and other health questionnaires.

Primary Objective: To evaluate treatment effect of 12-week Compound 1 administration in reducing alcohol consumption and promoting abstinence. Endpoint: Change from baseline in the proportion of Heavy Drinking Days (HDD) per week. HDD defined as ≥5 standard alcohol drinks for men and ≥4 standard alcohol drinks for women.

Secondary Objective 1: To assess the effect of 12 weeks Compound 1 administration versus placebo on other measures of alcohol use. Endpoints: (1) Change from baseline in the proportion of HDD in a month; (2) Change from baseline in the proportion of abstinent days per week; (3) Change in Total Alcohol Consumption from baseline to Week 12 based on the average daily alcohol consumption in grams per day (decrease in WHO drinking risk level >1 level and WHO drinking risk level >2 levels will be calculated); (4) Time to initial HDD; (5) Change from Baseline to week 12 in alcohol biomarkers of consumption and/or relapse (LFTs, GGT, % CDT and urine EtG); (6) Percentage of participants with no HDD per month or total abstinence; (7) Change from baseline in alcohol craving assessed by the Alcohol Craving Questionnaire-Short Form (ACQ-SF-R).

Secondary Objective 2: To assess the safety and tolerability of Compound 1 Endpoints: Vital signs, ECG parameters, clinical safety laboratory parameters (chemistry/hematology/urinalysis), (serious) AEs reporting, suicidal ideations (CSSR-S), BPRS, CADSS.

Secondary Objective 3: To evaluate the PK of Compound 1 in subjects with AUD. Endpoints: Plasma concentrations of Compound 1.

Exploratory Objective 1: To assess the frequency of other substances (cannabis, nicotine) before and during 12-week Compound 1 treatment. Endpoints: (1) TLFB cannabis and nicotine; (2) Urine test for cannabis; (3) Number of cigarette packs per day. (4) Fagerström Test for Nicotine Dependence and urine cotinine.

Exploratory Objective 2: To examine whether individual genetic variation in genes relating to drug metabolism and transporters, AUD, and the drug target pathway confirm differential response to Compound 1. Endpoints: Single genetic variant or genetic risk score and associations with outcome measures (pharmacogenetics blood samples).

Exploratory Objective 3: To assess the effect of 14-weeks Compound 1 administration versus placebo on: (a) Depressive symptoms; (b) Anxiety symptoms; (c) Quality of life; (e) Stress; (e) Sleep; (f) Global Functioning. Endpoints: (1) Patient Reports Outcomes (PRO) and wearable device data: (a) Beck's Depression Inventory (BDI-II); (b) State-Trait Anxiety Inventory (STAI) and digital assessment of heart rate variability; (c) 12-item Short Form Healthy Survey (SF-12); (d) Pittsburgh Sleep Quality Index (PSQI); (e) Short Inventory of Problems (SIP); (f) Digital assessment of electrodermal activity. (2) Clinician Reported Outcome: (g) CGI-S and CGI-I.

### Population

Key Inclusion Criteria: Patients who meet the following inclusion criteria will be eligible to participate in the study:

(1) Adult male or female subjects 18 to 65 years of age, inclusive.

(2) Has met the DSM-5 diagnosis (using the SCID-5-CT diagnostic interview) of at least a moderate AUD (4 or more criteria) for the 3-months immediately prior to signing the informed consent form.

(3) Must be seeking treatment for alcohol dependence and have a desire to reduce or cease alcohol use as per goals assessed at baseline.

(4) Must have at least 4 heavy drinking days per week over the past 4 weeks as assessed by the Timeline Follow Back (TLFB) scale at screening and baseline (heavy drinking is defined as greater than 4 drinks per day (or >40 g) for females and greater than 5 drinks (or >50 g) per day for males) [A standard drink is defined as 12 ounces (350 ml) of 5% beer, 5 ounces (150 ml) of 12% wine, or 1.5 ounces (44 ml) of 80-proof (40%) distilled spirits. Alcohol consumption will be converted from a number of standard drinks to grams by multiplying by 10, in accordance with WHO guidelines for estimating alcohol consumption-related harm (World Health Organization. International Guide for Monitoring Alcohol Consumption and Related Harm. WHO Press, Geneva, 2000).

(5) Must be abstinent from alcohol use for at least 3 days (but no more than 7 days) preceding first dosing (Day 1) as assessed by self-report TLFB, urinary EtG, and breathalyzer measurements (BAC by breathalyzer equal to 0.00 at baseline).

Key Exclusion Criteria: Patients who meet any of the following exclusion criteria will be excluded from participation in the study:

(1) Score “yes” on item 4 or item 5 of the Suicidal Ideation section of the C-SSRS, if this ideation occurred in the past 6 months, or “yes” on any item of the Suicidal Behavior section, except for the “Non-Suicidal Self-Injurious Behavior” (item also included in the Suicidal Behavior section) if this behavior occurred in the past 2 years.

(2) Have experienced an acute alcohol withdrawal syndrome (specifically hand tremors or seizures) within the prior 6 months.

(3) Have significant risk of an acute withdrawal syndrome in the opinion of the investigator or as determined by a score >10 on the CIWA-Ar (Wiehl W O, Hayner G, and Galloway G. Haight Ashbury free clinics' drug detoxification protocols—part 4: alcohol. Journal of Psychoactive Drugs (1994) 26:57-9. or has a history of prior alcohol withdrawal seizures or Delirium Tremens, or other conditions that would place the patient at increased risk of seizure.

(4) Has current diagnosis of moderate or severe substance use disorder (according to DSM-5) on alcohol, cannabis, opioids, or other stimulants, except alcohol. Note: Nicotine use is allowed. Current use of cannabis is allowed. Current use of amphetamine, cocaine and/or opiates is excluded, however, lifetime use is allowed).

(5) Meets current or lifetime DSM-5 criteria for current axis I disorder of major depression, panic disorder, obsessive-compulsive disorder, bipolar affective disorder, schizophrenia, or eating disorder or any other psychotic disorder or neurocognitive disorder except post-traumatic stress syndrome.

(6) Currently receiving treatment for substance use disorder (e.g., disulfiram, acamprosate, methylphenidate, modafinil, topiramate, immediate-release dexamphetamine, or baclofen) in the last 90 days.

(7) Requires treatment with any psychoactive medications, including any anti-seizure medications (with the exception of medications used for short-term treatment of insomnia). Note: SSRIs and benzodiazepines are allowed if they have adequate stable dose for at least 1 month prior to study treatment dosing.

### Dosage Forms and Route of Administration

Subjects will be assigned to one of the following 2 treatments arms in a 1:1 ratio. (1) Test product: Compound 1 capsules (dose to be determined); (2) Reference product: placebo capsules.

Administration route: Compound 1 or placebo will be administered orally.

### Evaluation Criteria

Efficacy/Pharmacodynamics: (1) Change from baseline in the proportion of Heavy Drinking Days (HDD) per week as assessed by the TLFB; (2) Alcohol Craving Questionnaire-Short Form (ACQ-SF-R); (3) Clinical Institute Withdrawal Assessment Alcohol Scale Revised (CIWA-AR); (4) Proportion of alcohol use days delivered from TLFB; (5) Alcohol consumption as delivered from TLFB.

Pharmacokinetics: (6) Compound 1 plasma concentrations pre- and post-morning dose.

Safety: (7) Physical examinations including vital signs, height, and weight; (8) Laboratory evaluations (chemistry/hematology/urinalysis); (9) Electrocardiograms (ECGs); (10) Suicidal ideation, and behavior as assessed by the Columbia-Suicide Severity Scale (C-SSRS); (11) Brief Psychotic Rating Scale (BPRS); (12) Serious adverse events (SAEs) and adverse events (AEs) reporting.

Other assessments: (13) Hair drug test for alcohol, amphetamine, methamphetamine, MDMA, MDEA, MDA, methylphenidate, ketamine) and Ethyl glucuronide (EtG); (14) Alcohol biomarkers (LFTs, % CDT, urine EtG, GGT); (15) TLFB Cannabis; (16) Number of cigarette packs per day; (17) Pharmacogenetics blood samples.

Patient Reported Outcome (PRO): (18) Beck's Depression Inventory (BDI-II); (19) State-Trait Anxiety Inventory (STAI); (20) 12-item Short Form Survey (SF-12); (21) Pittsburgh Sleep Quality Index (PSQI); (22) Short Inventory of Problems (SIP).

Clinician Reported Outcome (CRO): (23) Clinical Global Impression Scale-Severity (CGI-S) and Improvement (CGI-I); (24) Clinician-Administered Dissociative States Scale (CADSS);

Digital assessment via wearables/smartphone (Using passively collected data from smart device/sensor): (a) Total sleep duration and overall sleep quality; (b) Heart rate variability (HRV); (c) Electrodermal activity (EDA)

### Sample Size Determination

The sample size for this study will be approximately 70 subjects (35 subjects on Compound 1 and 35 subjects on placebo). This sample size will provide at least 80% power for a two-sided test with a level of significance a of 0.05 to detect a large effect size difference (0.84) in the proportion of heavy drinking days per week from baseline to Week 12 between Compound 1 and placebo groups.

1-39. (canceled)

40. A method of treating a subject having a substance use disorder (SUD), the method comprising administering to the subject a dosage of about 5 mg/day to about 100 mg/day of a compound of the formula 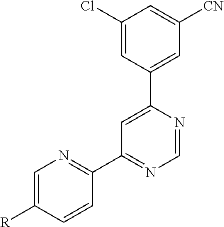
or a pharmaceutically acceptable salt thereof, wherein R is H, Me, F, Cl or cyano.

41. A method of treating a subject having a substance use disorder (SUD) who is at risk of relapse to substance use, the method comprising administering to the subject a therapeutically effective amount of a compound of the formula 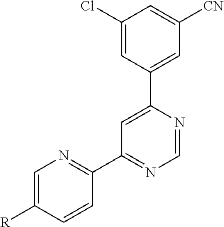
or a pharmaceutically acceptable salt thereof, wherein R is H, Me, F, Cl or cyano;
wherein the administration reduces susceptibility to one or more relapse triggers.

42. A method of promoting remission in a subject having a substance use disorder (SUD), the method comprising administering to the subject a therapeutically effective amount of a compound of the formula 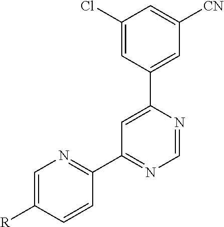
or a pharmaceutically acceptable salt thereof, wherein R is H, Me, F, Cl or cyano;
wherein the administration results in remission for at least 3 months.

43. The method of claim 40, wherein the administration results in remission of the SUD for at least 3 months.

44. The method of claim 40, wherein the administration prevents relapse for at least 3 months.

45. The method of claim 40, wherein the method further comprises the use of one or more tests to determine relapse.

46. The method of claim 45, wherein one of the one or more tests to determine relapse is a Timeline-Followback self-reported substance consumption assessment, a urine drug screening test, a saliva drug test, a hair follicle drug test, an acute alcohol ingestion test, or a chronic alcohol use test.

47. The method of claim 40, wherein the administration results in the subject having no early-relapse symptoms for at least 3 months.

48. The method of claim 40, wherein the method further comprises determining if the subject meets the DSM-5 diagnostic criteria of the SUD, administering the compound or the pharmaceutically acceptable salt thereof if the subject meets the DSM-5 criteria of the SUD, and not administering the compound or the pharmaceutically acceptable salt thereof if the subject does not meet the DSM-5 diagnostic criteria of the SUD.

49. The method of claim 40, wherein the compound or the pharmaceutically acceptable salt thereof is administered to the subject after a period of abstinence by the subject.

50. The method of claim 49, wherein the method further comprises performing a test to confirm abstinence.

51. The method of claim 49, wherein the period of abstinence is at least 12 hours and the subject has a breath or blood alcohol level of about 0.

52. The method of claim 49, wherein the period of abstinence is at least 1 month.

53. The method of claim 40, wherein the compound is 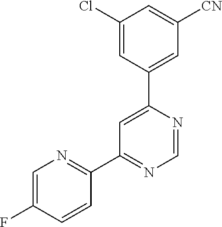 [SMILES: N#Cc1cc(Cl)cc(-c2cc(-c3ccc(F)cn3)ncn2)c1]
or a pharmaceutically acceptable salt thereof.

54. The method of claim 40, wherein the compound is 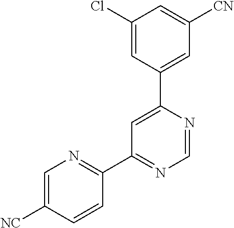 [SMILES: N#Cc1ccc(-c2cc(-c3cc(Cl)cc(C#N)c3)ncn2)nc1]
or a pharmaceutically acceptable salt thereof.

55. The method of claim 40, wherein the method achieves an average plasma concentration of about 10 ng/mL to about 300 ng/mL.

56. The method of claim 40, wherein the SUD is selected from the group consisting of alcohol use disorder (AUD), opioid use disorder (OUD), and a stimulant use disorder.

57. The method of claim 56, wherein the stimulant use disorder is cocaine use disorder (CUD).

58. The method of claim 41, wherein the subject is administered a dosage of about 5 mg/day to about 300 mg/day of the compound or the pharmaceutically acceptable salt thereof.

59. The method of claim 42, wherein the subject is administered a dosage of about 5 mg/day to about 300 mg/day of the compound or the pharmaceutically acceptable salt thereof.
